SEQUENCE LISTING (122 sequences)

SEQ ID NO: 1 - DNA, 28 bp, Artificial sequence
  Primer
cgttatccggatcacatgaaacggcatg

SEQ ID NO: 2 - DNA, 28 bp, Artificial sequence
  Primer
catgccgtttcatgtgatccggataacg

SEQ ID NO: 3 - DNA, 33 bp, Artificial sequence
  Synthetic DNA
agcttcatatgttcgaagtactagatctggtac

SEQ ID NO: 4 - DNA, 25 bp, Artificial sequence
  Synthetic DNA
cagatctagtacttcgaacatatga

SEQ ID NO: 5 - DNA, 109 bp, Artificial sequence
  Synthetic DNA
aattctataaaaataattgttgacatattttataaattttggcataatagatctaattgtgagcggataacaattctgcagaagcttgagctcggtacccggggatcct

SEQ ID NO: 6 - DNA, 109 bp, Artificial sequence
  Synthetic DNA
ctagaggatccccgggtaccgagctcaagcttctgcagaattgttatccgctcacaattagatctattatgccaaaatttataaaatatgtcaacaattatttttatag

SEQ ID NO: 7 - DNA, 77 bp, Artificial sequence
  Synthetic DNA
aggtaaccagcactatttaaagtccanccnccncgnccncgrtgngcnggngccatatgtatatctccttcttaaag

SEQ ID NO: 8 - DNA, 77 bp, Artificial sequence
  Synthetic DNA
aggtaaccagcactatttaaagtccanccnccyctnccyctrtgngcnggngccatatgtatatctccttcttaaag

SEQ ID NO: 9 - DNA, 24 bp, Artificial sequence
  Primer
ctttaagaaggagatatacatatg

SEQ ID NO: 10 - DNA, 27 bp, Artificial sequence
  Primer
ttcatacacggtgcctgactgcgttag

SEQ ID NO: 11 - DNA, 23 bp, Artificial sequence
  Primer
tcaacaagaattgggacaactcc

SEQ ID NO: 12 - DNA, 23 bp, Artificial sequence
  Primer
agcatatggcacctgctcacagg

SEQ ID NO: 13 - DNA, 27 bp, Artificial sequence
  Primer
ccctcggggcaggtcatccttggagag

SEQ ID NO: 14 - DNA, 77 bp, Artificial sequence
  Synthetic DNA
gggatgcttcgtggggtgtaggagatgcagcagtcagcactagtngcrtgraayctrtccatatgttggcgcgcctt

SEQ ID NO: 15 - DNA, 77 bp, Artificial sequence
  Synthetic DNA
gggatgcttcgtggggtgtaggagatgcagcagtcagcactagtngcrtgraancgrtccatatgttggcgcgcctt

SEQ ID NO: 16 - DNA, 18 bp, Artificial sequence
  Primer
aaggcgcgccaacatatg

SEQ ID NO: 17 - DNA, 69 bp, Artificial sequence
  Synthetic DNA
tgagcatgcatactagtctcgagtaatcccacagccgccgccagttccgctggcggcggcattttcgat

SEQ ID NO: 18 - DNA, 64 bp, Artificial sequence
  Synthetic DNA
cgaaaatgccgccgccagcggaactggcggcggctgtgggattactcgagactagtatgcatgc

SEQ ID NO: 19 - DNA, 20 bp, Artificial sequence
  Primer
tatggatcgatttcacgcaa

SEQ ID NO: 20 - DNA, 22 bp, Artificial sequence
  Synthetic DNA
ctagttgcgtgaaatcgatcca

SEQ ID NO: 21 - protein, 14 aa, Human
APAHRGRGGWTLNS

SEQ ID NO: 22 - DNA, 42 bp, Human
gcacctgctcacaggggacgaggaggctggaccctcaatagt

SEQ ID NO: 23 - DNA, 42 bp, Artificial sequence
  Synthetic DNA
gctccagcgcatcgtgggcgtggtggttggactttaaatagt

SEQ ID NO: 24 - DNA, 42 bp, Artificial sequence
  Synthetic DNA
gcacctgcacatcgtgggcgaggaggctggactttaaatagt

SEQ ID NO: 25 - DNA, 42 bp, Artificial sequence
  Synthetic DNA
gcaccacacgcacgcggacgagggggatggactttaaatagt

SEQ ID NO: 26 - DNA, 42 bp, Artificial sequence
  Synthetic DNA
gcaccagcacacagaggaaggggcggatggactttaaatagt

SEQ ID NO: 27 - DNA, 42 bp, Artificial sequence
  Synthetic DNA
gcaccagctcaccgaggccgaggaggctggactttaaatagt

SEQ ID NO: 28 - DNA, 42 bp, Artificial sequence
  Synthetic DNA
gcacccgctcaccgcggacgtggaggctggactttaaatagt

SEQ ID NO: 29 - DNA, 42 bp, Artificial sequence
  Synthetic DNA
gcaccagcacatagaggaagaggaggctggactttaaatagt

SEQ ID NO: 30 - DNA, 42 bp, Artificial sequence
  Synthetic DNA
gcaccggcacaccgcggacgagggggttggactttaaatagt

SEQ ID NO: 31 - DNA, 42 bp, Artificial sequence
  Synthetic DNA
gcaccggcgcaccgcggacgtggcagatggactttaaatagt

SEQ ID NO: 32 - DNA, 42 bp, Artificial sequence
  Synthetic DNA
gccccagcacatcgcggacgtggcggatggactttaaatagt

SEQ ID NO: 33 - DNA, 42 bp, Artificial sequence
  Synthetic DNA
gccccagcacaccgcggtcgcggcggttggactttaaatagt

SEQ ID NO: 34 - DNA, 42 bp, Artificial sequence
  Synthetic DNA
gctccagcacaccgggggcgggggggatggactttaaatagt

SEQ ID NO: 35 - DNA, 42 bp, Artificial sequence
  Synthetic DNA
gcaccggcacaccgcggccgcggaggatggactttaaatagt

SEQ ID NO: 36 - DNA, 42 bp, Artificial sequence
  Synthetic DNA
gcgccagctcatcggggtcgtggcgggtggactttaaatagt

SEQ ID NO: 37 - DNA, 42 bp, Artificial sequence
  Synthetic DNA
gctccagctcatcgaggacgcggcggttggactttaaatagt

SEQ ID NO: 38 - DNA, 42 bp, Artificial sequence
  Synthetic DNA
gcgccagcacaccgtggccgaggcggctggactttaaatagt

SEQ ID NO: 39 - DNA, 42 bp, Artificial sequence; identical to SEQ ID NO: 31
  Synthetic DNA

SEQ ID NO: 40 - DNA, 42 bp, Artificial sequence
  Synthetic DNA
gccccagcgcatcgggggcgtggcggctggactttaaatagt

SEQ ID NO: 41 - protein, 5 aa, Human
DRFHA

SEQ ID NO: 42 - DNA, 15 bp, Human
gacagattccacgct

SEQ ID NO: 43 - DNA, 15 bp, Artificial sequence
  Synthetic DNA
gacagattccacgca

SEQ ID NO: 44 - DNA, 15 bp, Artificial sequence
  Synthetic DNA
gacagattccacgcc

SEQ ID NO: 45 - DNA, 15 bp, Artificial sequence
  Synthetic DNA
gacagattccatgca

SEQ ID NO: 46 - DNA, 15 bp, Artificial sequence
  Synthetic DNA
gacagatttcacgca

SEQ ID NO: 47 - DNA, 15 bp, Artificial sequence
  Synthetic DNA
gacagatttcacgcc

SEQ ID NO: 48 - DNA, 15 bp, Artificial sequence
  Synthetic DNA
gacagatttcacgcg

SEQ ID NO: 49 - DNA, 15 bp, Artificial sequence
  Synthetic DNA
gacaggttccacgca

SEQ ID NO: 50 - DNA, 15 bp, Artificial sequence
  Synthetic DNA
gacagattccacgcg

SEQ ID NO: 51 - DNA, 15 bp, Artificial sequence
  Synthetic DNA
gacaggtttcacgct

SEQ ID NO: 52 - DNA, 15 bp, Artificial sequence
  Synthetic DNA
gacaggtttcatgcc

SEQ ID NO: 53 - DNA, 15 bp, Artificial sequence
  Synthetic DNA
gaccgattccacgcg

SEQ ID NO: 54 - DNA, 15 bp, Artificial sequence
  Synthetic DNA
gaccgattccacgct

SEQ ID NO: 55 - DNA, 15 bp, Artificial sequence
  Synthetic DNA
gaccgattccatgcc

SEQ ID NO: 56 - DNA, 15 bp, Artificial sequence
  Synthetic DNA
gaccgattccatgcg

SEQ ID NO: 57 - DNA, 15 bp, Artificial sequence
  Synthetic DNA
gaccgattccatgct

SEQ ID NO: 58 - DNA, 15 bp, Artificial sequence
  Synthetic DNA
gaccgatttcatgcc

SEQ ID NO: 59 - DNA, 15 bp, Artificial sequence
  Synthetic DNA
gaccgcttccacgcc

SEQ ID NO: 60 - DNA, 15 bp, Artificial sequence
  Synthetic DNA
gaccgcttccacgcg

SEQ ID NO: 61 - DNA, 15 bp, Artificial sequence
  Synthetic DNA
gaccgcttccacgct

SEQ ID NO: 62 - DNA, 15 bp, Artificial sequence
  Synthetic DNA
gaccgctttcacgca

SEQ ID NO: 63 - DNA, 15 bp, Artificial sequence
  Synthetic DNA
gaccggttccacgca

SEQ ID NO: 64 - DNA, 15 bp, Artificial sequence
  Synthetic DNA
gaccggttccatgcc

SEQ ID NO: 65 - DNA, 15 bp, Artificial sequence
  Synthetic DNA
gaccggtttcacgca

SEQ ID NO: 66 - DNA, 15 bp, Artificial sequence
  Synthetic DNA
gatagattccacgct

SEQ ID NO: 67 - DNA, 15 bp, Artificial sequence
  Synthetic DNA
gatagatttcacgcc

SEQ ID NO: 68 - DNA, 15 bp, Artificial sequence
  Synthetic DNA
gatagatttcacgcg

SEQ ID NO: 69 - DNA, 15 bp, Artificial sequence
  Synthetic DNA
gatagatttcacgct

SEQ ID NO: 70 - DNA, 15 bp, Artificial sequence
  Synthetic DNA
gataggttccacgct

SEQ ID NO: 71 - DNA, 15 bp, Artificial sequence
  Synthetic DNA
gatcgatttcacgca

SEQ ID NO: 72 - DNA, 15 bp, Artificial sequence
  Synthetic DNA
gatcgcttccacgca

SEQ ID NO: 73 - DNA, 15 bp, Artificial sequence
  Synthetic DNA
gatcgctttcacgct

SEQ ID NO: 74 - DNA, 15 bp, Artificial sequence
  Synthetic DNA
gatcggttccacgca

SEQ ID NO: 75 - DNA, 63 bp, Artificial sequence
  Synthetic DNA
ctagcgaattcgagcatatgagcactagtgcatgcgagccatattcaacgggaaacgtcttgc

SEQ ID NO: 76 - DNA, 63 bp, Artificial sequence
  Synthetic DNA
tcgagcaagacgtttcccgttgaatatggctcgcatgcactagtgctcatatgctcgaattcg

SEQ ID NO: 77 - DNA, 24 bp, Artificial sequence
  Synthetic DNA
tatgagggtaccgccggctgcatg

SEQ ID NO: 78 - DNA, 18 bp, Artificial sequence
  Synthetic DNA
cagccggcggtaccctca

SEQ ID NO: 79 - DNA, 26 bp, Artificial sequence
  Synthetic DNA
tatgagggtaccgccggcactgcatg

SEQ ID NO: 80 - DNA, 20 bp, Artificial sequence
  Synthetic DNA
cactgccggcggtaccctca

SEQ ID NO: 81 - DNA, 30 bp, Artificial sequence
  Synthetic DNA
ctcatatggctgtgatcaccggtgcctgtg

SEQ ID NO: 82 - DNA, 30 bp, Artificial sequence
  Synthetic DNA
gcggatccctaaaaattgatgttcttcaag

SEQ ID NO: 83 - DNA, 88 bp, Artificial sequence
  Synthetic DNA
tatggcggtgattaccggtgcgtgcgaacgtgatgtgcagtgcggtgcgggtacctgctgcgcgattagcctgtggctgcgtggtctg

SEQ ID NO: 84 - DNA, 92 bp, Artificial sequence
  Synthetic DNA
cgtatgtgcaccccgctgggtcgtgaaggtgaagaatgccatccgggtagccataaagtgccgttcttccgtaaacgtaaacatcatacctg

SEQ ID NO: 85 - DNA, 86 bp, Artificial sequence
  Synthetic DNA
cccgtgcctgccgaacctgctgtgcagccgtttcccggatggtcgttatcgttgcagcatggatctgaaaaacattaacttttagg

SEQ ID NO: 86 - DNA, 94 bp, Artificial sequence
  Synthetic DNA
cacatacgcagaccacgcagccacaggctaatcgcgcagcaggtacccgcaccgcactgcacatcacgttcgcacgcaccggtaatcaccgcca

SEQ ID NO: 87 - DNA, 93 bp, Artificial sequence
  Synthetic DNA
aggcacgggcaggtatgatgtttacgtttacggaagaacggcactttatggctacccggatggcattcttcaccttcacgacccagcggggtg

SEQ ID NO: 88 - DNA, 81 bp, Artificial sequence
  Synthetic DNA
gatccctaaaagttaatgtttttcagatccatgctgcaacgataacgaccatccgggaaacggctgcacagcaggttcggc

SEQ ID NO: 89 - DNA, 85 bp, Artificial sequence
  misc_difference 46: n is a, g, t or c
agagtttgccggcaccggtccggtacccgcaccgcactgcacrtcncgytcrcangcnccngtdatnacngccatatgttggcgc

SEQ ID NO: 90 - DNA, 85 bp, Artificial sequence
  misc_difference 55: n is a, g, t or c
agagtttgccggcaccggtccggtacccgcaccgcactgcacrtcyctytcrcangcnccngtdatnacngccatatgttggcgc

SEQ ID NO: 91 - DNA, 13 bp, Artificial sequence
  Synthetic DNA
gcgccaacatatg

SEQ ID NO: 92 - DNA, 23 bp, Artificial sequence
  Synthetic DNA
acctgattgcccgacattatcgc

SEQ ID NO: 93 - DNA, 23 bp, Artificial sequence
  Synthetic DNA
caccctcatcagtgccaacatag

SEQ ID NO: 94 - DNA, 20 bp, Artificial sequence
  Synthetic DNA
taatacgactcactataggg

SEQ ID NO: 95 - DNA, 32 bp, Artificial sequence
  Synthetic DNA
atacatggatcctcaggtcatccttggagagc

SEQ ID NO: 96 - DNA, 100 bp, Artificial sequence
  Synthetic DNA
tatggctccagcgcatcgtgggcgtggtggttggactttaaatagtgctggatacttgttaggtccagtgttacatttaagcagcaaagcaaaccagggc

SEQ ID NO: 97 - DNA, 88 bp, Artificial sequence
  Synthetic DNA
aggaagacagactcagctcttgagatcctagatctgtggaaggccatagatgggcttccttattctcggtctccccgcatgacgtgag

SEQ ID NO: 98 - DNA, 90 bp, Artificial sequence
  Synthetic DNA
ttgctttgctgcttaaatgtaacactggacctaacaagtatccagcactatttaaagtccaaccaccacgcccacgatgcgctggtgcca

SEQ ID NO: 99 - DNA, 100 bp, Artificial sequence
  Synthetic DNA
gatcctcacgtcatgcggggagaccgagaataaggaagcccatctatggccttccacagatctaggatctcaagagctgagtctgtcttcctgccctggt

SEQ ID NO: 100 - DNA, 30 bp, Artificial sequence
  Synthetic DNA
ggaattcgctcgcaccggtagaaaaaatgg

SEQ ID NO: 101 - DNA, 36 bp, Artificial sequence
  Synthetic DNA
ctagccattttttctaccggtgcgagcgaattccgc

SEQ ID NO: 102 - DNA, 55 bp, Artificial sequence
  Synthetic DNA
aattccgtcgtacaccatgggcctccacctccgcccctaccgtgtggggctgctc

SEQ ID NO: 103 - DNA, 55 bp, Artificial sequence
  Synthetic DNA
ccgggagcagccccacacggtaggggcggaggtggaggcccatggtgtacgacgg

SEQ ID NO: 104 - DNA, 55 bp, Artificial sequence
  Synthetic DNA
aattccgtcgtacaccatgggcctgcacctgcggccctaccgggtgggcctgctg

SEQ ID NO: 105 - DNA, 55 bp, Artificial sequence
  Synthetic DNA
ccggcagcaggcccacccggtagggccgcaggtgcaggcccatggtgtacgacgg

SEQ ID NO: 106 - DNA, 55 bp, Artificial sequence
  Synthetic DNA
aattccgtcgtacaccatgggacttcaccttagaccttacagagtgggacttctt

SEQ ID NO: 107 - DNA, 55 bp, Artificial sequence
  Synthetic DNA
ccggaagaagtcccactctgtaaggtctaaggtgaagtcccatggtgtacgacgg

SEQ ID NO: 108 - DNA, 55 bp, Artificial sequence
  Synthetic DNA
aattccgtcgtacaccatgggtctacatctacgtccgtatcgtgtaggtctacta

SEQ ID NO: 109 - DNA, 55 bp, Artificial sequence
  Synthetic DNA
ccggtagtagacctacacgatacggacgtagatgtagacccatggtgtacgacgg

SEQ ID NO: 110 - DNA, 55 bp, Artificial sequence
  Synthetic DNA
aattccgtcgtacaccatggggctacatctacgcccgtatcgcgtagggctacta

SEQ ID NO: 111 - DNA, 55 bp, Artificial sequence
  Synthetic DNA
ccggtagtagccctacgcgatacgggcgtagatgtagccccatggtgtacgacgg

SEQ ID NO: 112 - DNA, 55 bp, Artificial sequence
  Synthetic DNA
aattccgtcgtacaccatgggtctacatcttagaccctatcgtgtaggactgctt

SEQ ID NO: 113 - DNA, 55 bp, Artificial sequence
  Synthetic DNA
ccggaagcagtcctacacgatagggtctaagatgtagacccatggtgtacgacgg

SEQ ID NO: 114 - DNA, 55 bp, Artificial sequence
  Synthetic DNA
aattccgtcgtacaccatgggtctacatcttagaccctaccgagttggattgctt

SEQ ID NO: 115 - DNA, 55 bp, Artificial sequence
  Synthetic DNA
ccggaagcaatccaactcggtagggtctaagatgtagacccatggtgtacgacgg

SEQ ID NO: 116 - protein, 23 aa, Human
WYKHVASPRYHTVGRAAGLLMGL

SEQ ID NO: 117 - protein, 86 aa, Human
AVITGACERDVQCGAGTCCAISLWLRGLRMCTPLGREGEECHPGSHKVPFFRKRKHHTCPCLPNLLCSRFPDGRYRCSMDLKNINF

SEQ ID NO: 118 - protein, 146 aa, Human
PALPEDGGSGAFPPGHFKDPKRLYCKNGGFFLRIHPDGRVDGVREKSDPHIKLQLQAEERGVVSIKGVSANRYLAMKEDGRLLASKSVTDECFFFERLESNNYNTYRSRKYTSWYVALKRTGQYKLGSKTGPGQKAILFLPMSAKS

SEQ ID NO: 119 - DNA, 180 bp, Artificial sequence
  Synthetic DNA
gctccagcgcatcgtgggcgtggtggttggactttaaatagtgctggttacctcctgggtcctgtcctccacctttcctcaaaggccaaccagggcaggaagacagactcagctcttgagatcctagacctgtggaaggccatagatgggctcccttattcccgctctccaaggatgacc

SEQ ID NO: 120 - DNA, 228 bp, Artificial sequence
  Synthetic DNA
gatcgatttcacgcaactagtgctgactgctgcatctcctacaccccacgaagcatcccgtgttcactcctggagagttactttgaaacgaacagcgagtgctccaagccgggtgtcatcttcctcaccaagaaggggcgacgtttctgtgccaaccccagtgataagcaagttcaggtttgcatgagaatgctgaagctggacacacggatcaagaccaggaagaat

SEQ ID NO: 121 - DNA, 258 bp, Artificial sequence
  Synthetic DNA
gcggtgattaccggtgcgtgcgaacgtgatgtgcagtgcggtgcgggtacctgctgcgcgattagcctgtggctgcgtggtctgcgtatgtgcaccccgctgggtcgtgaaggtgaagaatgccatccgggtagccataaagtgccgttcttccgtaaacgtaaacatcatacctgcccgtgcctgccgaacctgctgtgcagccgtttcccggatggtcgttatcgttgcagcatggatctgaaaaacattaacttt

SEQ ID NO: 122 - DNA, 180 bp, Artificial sequence
  Synthetic DNA
gctccagcgcatcgtgggcgtggtggttggactttaaatagtgctggatacttgttaggtccagtgttacatttaagcagcaaagcaaaccagggcaggaagacagactcagctcttgagatcctagatctgtggaaggccatagatgggcttccttattctcggtctccccgcatgacg

(19) United States
(12) Patent Application Publication
(10) Pub. No.: US 2005/0209447 A1
(43) Pub. Date: Sep. 22, 2005

(54) Process for producing recombinant protein

(76) Inventors: Takashi Ito, Kobe-shi (JP); Yoko Tanaka, Kyotanabe-shi (JP); Mitsuyo Kondo, Kawanishi-shi (JP)

Correspondence Address: TAKEDA PHARMACEUTICALS NORTH AMERICA, INC;INTELLECTUAL PROPERTY DEPARTMENT, 475 HALF DAY ROAD, SUITE 500, LINCOLNSHIRE, IL, 60069, US

(21) Appl. No.: 10/343,095

(22) PCT Filed: Jul. 25, 2001
(86) PCT No.: PCT/JP01/06392

§ 371 Date: Jan. 24, 2003

(30) Foreign Application Priority Data
Jul. 25, 2000 (JP) 2000-229064

(51) Int. Cl. C07H 21/04; C12P 21/06; C12N 15/09

(52) U.S. Cl. 536/23.5; 435/69.1; 435/320.1; 435/325; 530/350

(57) ABSTRACT

It is intended to produce a target protein in procaryotic or eucaryotic cells. A DNA encoding the full-length amino acid sequence or a part of the same of a target protein is prepared and the target protein is produced by a gene recombination technique with the use of this DNA.

### TECHNICAL FIELD

The present invention relates to a process for producing proteins. More specifically, the present invention relates to a process for producing a protein of interest in a prokaryotic or eukaryotic cell, as well as a process for preparing a DNA that encodes a protein of interest and is suitable for expression in a prokaryotic or eukaryotic cell.

### BACKGROUND ART

Various attempts have been made to produce useful proteins in procaryotic or eukaryotic cells using recombinant DNA technology. It is very difficult to produce proteins with relatively high molecular weights by chemical synthesis. On the other hand, proteins with low molecular weights can be produced efficiently in the form of a fusion protein using recombinant DNA technology. Expression of useful proteins using prokaryotes, especially Escherichia coli, can be performed rapidly and cheaply. However, depending on the type of the protein to be expressed by recombinant DNA technology, there are some cases where expression is hardly recognized or, even if expressed, the expression level is very low.

When a protein of interest is expressed in a heterologous organism (e.g., a protein of a eukaryote is expressed in a prokaryote), codon usage is different; or when a secretion protein from eukaryotes is expressed in prokaryotes, only the mature protein moiety is expressed in many cases excluding the signal peptide moiety at which translation originally starts in eukaryotes. Thus, there arise such problems that a protein highly expressed in eukaryotes is not expressed in prokaryotes or, even if expressed, the expression level is extremely low. Further, even when a protein of interest is expressed in a host similar to the original organism in species (e.g., a human-derived protein is expressed in an animal cell), he expression level may differ greatly depending on the type of the protein. Development of a technology to allow the high expression of proteins of interest in prokaryotic and eukaryotic cells using recombinant DNA technology is extremely useful from an industrial point of view.

### DISCLOSURE OF THE INVENTION

The present inventors have found that the difference in expression levels when a useful protein is produced in a prokaryotic or eukaryotic cell using recombinant DNA technology is mainly attributable to the sequence of the DNA encoding the protein. As a result of further researches, the inventors have achieved the present invention.

The present invention relates to:
(1) A process of producing a DNA that encodes a protein of interest and is suitable for expression, comprising the following steps:
1) preparing recombinant vectors by using a mixture of a plurality of DNAs encoding the full length or a partial amino acid sequence of the protein of interest and integrating each of the DNAs into a vector retaining a promoter DNA functional in a host cell and a reporter gene so that the DNA is located downstream of the promoter DNA and upstream of the reporter gene,
2) transforming the host cell with the recombinant vectors,
3) culturing the resultant transformants,
4) measuring the expression of the reporter gene, and
5) preparing a DNA that comprises the same nucleotide sequence as that of the DNA encoding the full length or the partial amino acid sequence of the protein of interest possessed by a transformant in which a high expression of the reporter gene has been confirmed and that encodes the protein of interest;
(2) The production process of (1) above, wherein the full length or a partial amino acid sequence of the protein of interest is the full length of the protein of interest or a partial amino acid sequence comprising the N-terminal of the protein of interest;
(3) A process of producing a protein of interest or a salt thereof, comprising the following steps:
1) preparing recombinant vectors by using a mixture of a plurality of DNAs encoding the full length or a partial amino acid sequence of the protein of interest and integrating each of the DNAs into a vector retaining a promoter DNA functional in a host cell and a reporter gene so that the DNA is located downstream of the promoter DNA and upstream of the reporter gene,
2) transforming the host cell with the recombinant vectors,
3) culturing the resultant transformants,
4) measuring the expression of the reporter gene,
5) preparing a recombinant vector comprising a DNA that comprises the same nucleotide sequence as that of the DNA encoding the full length or the partial amino acid sequence of the protein of interest possessed by a transformant in which a high expression of the reporter gene has been confirmed and that encodes the protein of interest,
6) transforming a host cell with the recombinant vector, and
7) culturing the resultant transformant;
(4) The production process of (2) above, wherein the full length or a partial amino acid sequence of the protein of interest is the full length of the protein of interest or a partial amino acid sequence comprising the N-terminal of the protein of interest;
(5) A process of producing a DNA that encodes a eukaryote-derived protein of interest and is suitable for expression in a prokaryote, comprising the following steps:
1) preparing recombinant vectors by using a mixture of a plurality of DNAs encoding an N-terminal amino acid sequence of the eukaryote-derived protein of interest and integrating each of the DNAs into a vector retaining a promoter DNA functional in prokaryotes and a reporter gene-so that the DNA is located downstream of the promoter DNA and upstream of the reporter gene,
2) transforming the prokaryote with the recombinant vectors,
3) culturing the resultant prokaryotic transformants,
4) measuring the expression of the reporter gene, and
5) preparing a DNA that comprises the same nucleotide sequence as that of the DNA encoding the N-terminal amino acid sequence of the protein of interest possessed by a transformant in which a high expression of the reporter gene has been confirmed and that encodes the protein of interest;
(6) A process of producing a eukaryote-derived protein of interest or a salt thereof in a prokaryote, comprising the following steps:
1) preparing recombinant vectors by using a mixture of a plurality of DNAs encoding an N-terminal amino acid sequence of the eukaryote-derived protein of interest and integrating each of the DNAs into a vector retaining a promoter DNA functional in prokaryotes and a reporter gene so that the DNA is located downstream of the promoter DNA and upstream of the reporter gene,
2) transforming the prokaryote with the recombinant vectors,
3) culturing the resultant prokaryotic transformants,
4) measuring the expression of the reporter gene,
5) preparing a recombinant vector comprising a DNA that comprises the same nucleotide sequence as that of the DNA encoding the N-terminal amino acid sequence of the protein of interest possessed by a transformant in which a high expression of the reporter gene has been confirmed and that encodes the protein of interest,
6) transforming the prokaryote with the recombinant vector, and
7) culturing the resultant prokaryotic transformant;
(7) A DNA obtained by the production process of (1) above that comprises the same nucleotide sequence as that of the DNA encoding the full length or a partial amino acid sequence of a protein of interest possessed by a transformant in which a high expression of the reporter gene has been confirmed and that encodes the full length of the protein of interest;
(8) A non-wild type DNA obtained by the production process of (1) above encoding the full length of a protein of interest, wherein the DNA is composed of a DNA encoding a partial amino acid sequence of the protein of interest possessed by a transformant in which a high expression of the reporter gene has been confirmed and a DNA(s) encoding the remaining amino acid sequence(s) of the protein ligated to the 3′ end or/and 5′ end of the DNA;
(9) A DNA having the nucleotide sequence as shown in SEQ ID NO: 119, SEQ ID NO: 120, SEQ ID NO: 121 or SEQ ID NO: 122;
(10) A recombinant vector comprising the DNA of any one of (7) to (9) above;
(11) A transformant transformed with the recombinant vector of (10) above; and
(12) A process of producing a protein of interest or a salt thereof, comprising culturing the transformant of (I1) above and allowing the transformant to produce the protein of interest.

### BRIEF DESCRIPTION OF THE DRAWINGS

FIG. 1 shows a construction diagram for plasmid pGFPNP.

FIG. 2 shows a construction diagram for plasmid pGFPNPRG.

FIG. 3 shows a construction diagram for plasmid pTFCRGAL.

FIG. 4 shows a construction diagram for plasmid pTFCS4.

FIG. 5 shows the growth curves of E. coli MM294 (DE3)/pTFCRGAL and E. coli MM294 (DE3)/pTFCS4.

FIG. 6 shows the results of analysis of the products from E. coli MM294 (DE3)/pTFCRGAL and E. coli MM294 (DE3)/pTFCS4.

FIG. 7 shows a construction diagram for plasmid pGFPNPMP.

FIG. 8 shows a construction diagram for plasmid pNPHGHlacts.

FIG. 9 shows a construction diagram for plasmid pNPMPIF.

FIG. 10 shows a construction diagram for plasmid pNPMPIFNO.

FIG. 11 shows the fluorescence intensities of the E. coli clones obtained in Example 6.

FIG. 12 shows the growth curves and MPIF1Δ23 expression levels of E. coli JM109/pNPMPIF retaining plasmid pNPMPIF having the wild-type nucleotide sequence and E. coli JM109/pNPMPIFNO retaining plasmid pNPMPIFNO having a modified nucleotide sequence. In this Figure, mark ◯ represents the growth curve, and mark ● represents the MPIF1Δ23 expression level.

FIG. 13 shows a construction diagram for plasmid pKANP.

FIG. 14 shows the amino acid sequence and the wild-type nucleotide sequence of ZAQ-1 ligand.

FIG. 15 is a construction diagram for plasmid pTCIIZAQN.

FIG. 16 shows the amino acid sequence, the wild-type nucleotide sequence and a nucleotide sequence comprising highly frequently used codons, of ZAQ-1 ligand.

FIG. 17 shows the synthetic DNAs used for the construction of a ZAQ-1 structural gene using highly frequently used codons.

FIG. 18 shows a construction diagram for plasmid pTCIIZAQS.

FIG. 19 shows a construction diagram for plasmid pKANPZAQ.

FIG. 20 shows the nucleotide sequence obtained in Example 14.

FIG. 21 shows a construction diagram for plasmid pTCIIZAQNO.

FIG. 22 shows the results of the expression analysis carried out in Example 16.

FIG. 23 shows a construction diagram for plasmid pTCRGDE.

FIG. 24 shows a construction diagram for plasmid pTCRGNODE.

FIG. 25 shows the rat GALP full length-optimized nucleotide sequence obtained in Example 19.

FIG. 26 shows a construction diagram for plasmid pTCRGTODE.

FIG. 27 shows the results of SDS-PAGE obtained in Example 21.

FIG. 28 shows a construction diagram for plasmid pQBI25-3.

FIG. 29 shows the nucleotide sequences encoding an N-terminal portion of LLPL used in Example 23.

FIG. 30 shows the results of the fluorescence measurement carried out in Example 24.

FIG. 31 shows the results of the analysis carried out in Example 25.

### BEST MODES FOR CARRYING OUT THE INVENTION

Codons encoding the individual amino acids composing proteins are as described below.
Phe: TTY (TTT or TTC)
Leu: TTR or CTN (TTA, TTG, CTT, CTC, CTA or CTG)
Ile: ATH (ATT, ATC or ATA)
Met: ATG
Val: GTN (GTT, GTC, GTA or GTG)
Ser: TCN or AGY (TCT, TCC, TCA, TCG, AGT, AGC)
Pro: CCN (CCT, CCC, CCA or CCG)
Thr: ACN (ACT, ACC, ACA or ACG)
Ala: GCN (GCT, GCC, GCA or GCG)
Tyr: TAY (TAT or TAC)
His: CAY (CAT or CAC)
Gln: CAR (CAA or CAG)
Asn: AAY (AAT or AAC)
Lys: AAR (AAA or AAG)
Asp: GAY (GAT or GAC)
Glu: GAR (GAA or GAG)
Cys: TGY (TGT or TGC)
Trp: TGG
Arg: CGN or AGR (CGT, CGC, CGA, CGG, AGA or AGG)
Gly: GGN (GGT, GGC, GGA or GGG)
Stop codon: TAR or TGA (TAA, TAG or TGA)

In the present specification, a protein of interest is written with the N-terminal (amino terminal) on the left end and the C-terminal (carboxyl terminal) on the right end according to the convention in peptide representation.

As a protein of interest, a protein that may be used in drugs, reagents, raw materials, feeds, etc. is used. More specifically, a eukaryote-derived protein having physiological activity is used as a protein of interest. Specific examples of such a protein include growth factors, interleukins, interferons, chemokines, and physiologically active peptides [e.g. KiSS-1 peptide (WO 00/24890), RFRP-1 (WO 00/29441), apelin (WO 99/33976), PrRP (WO 97/24436), GALP (WO 99/48920), GPR8 ligand, ZAQ-1 ligand, angiotensin, bradykinin, calcitonin, conantokin, corticotropin-releasing factor, dynorphin, endorphin, enkephalin, galanin, GALP (ligand peptide for rat galanin receptor; WO 99/48920), MPIF-1Δ23 (myeloid progenitor inhibitory factor 1Δ23; WO 95/17092) gastrin, glucagon, growth hormone-releasing factor, FMRF-amide, neurokinin, neuromedin, neuropeptide, nociceptin, nocistatin, orexin-B, selectin, substance P. urocortin, VIP, PACAP, ACTH, various opioids).

As GPR8 ligand mentioned above, any polypeptide may be used as long as it has ligand activity for a 7-transmembrane type receptor protein GPR8 (O'Dowd, B. F. et al., Genomics, 28:84-91, 1995) (e.g. GPR8 binding activity) or cell stimulating activity against GPR-expressing cells (e.g. activities to promote arachidonate liberation, acetylcholine liberation, intracellular Ca²⁺ liberation, intracellular cAMP production, intracellular cGMP production, inositol phosphate production, cellular membrane voltage variation, phposphorylation of intracellular proteins, c-fos activation, pH lowering, GTP γ S binding activity, etc.). Preferably, a peptide having the amino acid sequence as shown in SEQ ID NO: 116 may be used.

As ZAQ-1 ligand mentioned above, ligands for ZAQ receptor may be used. Specifically, a peptide having the amino acid sequence as shown in SEQ ID NO: 117 or the like may be used.

These proteins may be used alone in their intrinsic forms. Alternatively, they may be used in a form where another eukaryote-derived protein, a prokaryote-derived protein or a tag such as polyhistidine is fused to the C-terminal.

As a eukaryote-derived protein to be fused to the C-terminal, a fibroblast growth factor (aFGF, bFGF, etc.) or a mutein thereof, or a part (fragment) of such a factor or its mutein (e.g. bFGF CS23 mutein etc.) may be used preferably.

As bFGF CS23 mutein, a protein having the amino acid sequence as shown in SEQ ID NO: 118 may be used, for example.

A protein of interest may be in the form of a salt. As a salt of a protein of interest, a physiologically acceptable acid-addition salt is particularly preferable. Examples of such salts useful in the invention include salts formed with inorganic acids (e.g. hydrochloric acid, phosphoric acid, hydrobromic acid or sulfuric acid) and salts formed with organic acids (e.g. acetic acid, formic acid, propionic acid, fumaric acid, maleic acid, succinic acid, tartaric acid, citric acid, malic acid, oxalic acid, benzoic acid, methanesulfonic acid or benzenesulfonic acid).

As a reporter gene, such a gene may be used for which the presence or absence of expression can be confirmed easily. Specific examples of reporter genes include genes of fluorescent proteins such as green fluorescent protein(GFP) gene, enzyme genes such as alkali phosphatase gene, β-galactosidase gene, dihydrofolate reductase gene, and drug resistance genes such as kanamycin resistance gene, chloramphenicol resistance gene, neomycin resistance gene, hygromycin resistance gene. One or a plurality (preferably, two or three) of these genes may be used. Among all, green fluorescent protein (GFP) gene is preferable.

By using a plurality of codons encoding a single amino acid, a mixture of a plurality of DNAs encoding the same amino acid sequence is obtained. A plurality of DNAs encoding the full length or a partial amino acid sequence of a protein of interest can be synthesized chemically according to conventional methods using mixed bases encoding amino acids composing the relevant amino acid sequence.

The full-length amino acid sequence of a protein of interest refers to an amino acid sequence encoding the full length of the protein of interest.

A partial amino acid sequence of a protein of interest refers to any partial amino acid sequence of the full-length amino acid sequence of the protein of interest. Usually, an amino acid sequence consisting of about 5 or more, preferably about 5-50, more preferably about 5-15, still more preferably about 5-10 amino acid residues is used. A preferable partial amino acid sequence is a partial amino acid sequence encoding an N-terminal portion of the full-length amino acid sequence of a protein of interest; usually, an amino acid sequence consisting of about 5 or more, preferably about 5-50, more preferably about 5-15, still more preferably about 5-10 amino acid residues counted from the N-terminal is preferably used. In particular, an amino acid sequence spanning from the N-terminal to about the 15th amino acid residue, preferably from the N-terminal to about the 10th amino acid residue is used.

It is preferable to provide DNA fragments with different restriction sites at the 5′ ends and the 3′ ends in order to avoid ligation among DNA fragments.

By using a mixture of a plurality of DNAs and integrating each of the DNA into a vector retaining a promoter DNA functional in a host cell and a reporter gene downstream of the promoter DNA and upstream of the reporter gene, a mixture of a plurality of recombinant vectors for confirming the expression of a reporter gene can be obtained.

By transforming a host cell with the resultant mixture of recombinant vectors, a plurality of transformants are obtained.

The resultant mixture of a plurality of transformants is cultured, followed by confirmation of the presence or absence of the expression of the reporter gene.

When the host is a prokaryote, the expression levels of the reporter gene in individual transformants can be easily confirmed by coating the mixture of transformants on a solid medium, such as agar medium, and culturing them. Also, a single clone can be easily isolated. The expression levels of the reporter gene can be measured by determining the amounts of the reporter gene product. For example, when GFP gene is used as a reporter gene, the amount of GFP can be determined by exciting at the wavelength of 350-500 and measuring the fluorescence intensity at 450-550 nm. When a drug resistance gene is used as a reporter gene, only those transformants with high expression of the reporter gene can be grown by coating a mixture of transformants on a solid medium (such as agarose medium) containing the relevant drug at an appropriate concentration. When an enzyme gene is used as a reporter gene, enzyme activities produced by transformants can be measured by conventional methods.

When the host is a eukaryotic cell, if GFP gene is used as a reporter gene, it is possible to obtain those clones with high expression of GFP gene by culturing a mixture of transformants and utilizing the cell sorting function of a flowcytometer. Also, it is possible to obtain a single cell by limiting dilution, culture the cell and then determining the expression level of the reporter gene by measuring the fluorescence intensity or the activity of the enzyme produced by the transformant according to known methods.

When the host is a prokaryote, a recombinant vector can be extracted by conventional methods from the transformant (clone) in which expression of the reporter gene has been confirmed. By digesting the extracted recombinant vector with appropriate restriction enzymes, a DNA fragment can be obtained that is suitable for the production of the protein of interest in a prokaryote and encodes the full length or a partial amino acid sequence of the protein of interest. If necessary, the nucleotide sequence of the resultant DNA fragment may be confirmed. Further, by amplifying by colony PCR a DNA encoding an N-terminal amino acid sequence of the protein of interest from a transformant (clone) in which expression of the reporter gene has been confirmed and then digesting the DNA with appropriate restriction enzymes, a DNA fragment can be obtained that is suitable for the production of the protein of interest in a prokaryote and encodes the N-terminal amino acid sequence of the protein of interest. If necessary, the nucleotide sequence of the resultant DNA fragment may be confirmed. Also, a DNA comprising the same nucleotide sequence as that of the DNA fragment obtained and encoding the full length or a partial amino acid sequence of the protein of interest may be synthesized chemically.

When the host is a eukaryotic cell, mRNA may be extracted by conventional methods from the transformant (clone) in which expression of the reporter gene has been confirmed. By preparing cDNA from the extracted mRNA using a reverse transcriptase, a DNA fragment encoding the full length or a partial amino acid sequence of a protein of interest can be obtained. If necessary, the nucleotide sequence of the resultant DNA fragment may be confirmed. Further, by extracting DNA from the transformant (clone) by conventional methods, amplifying the DNA by PCR and then digesting with appropriate restriction enzymes, it is possible to obtain a DNA fragment that is suitable for production of the protein of interest and encodes an N-terminal amino acid sequence of the protein of interest. If necessary, the nucleotide sequence of the resultant DNA fragment may be confirmed. Also, a DNA comprising the same nucleotide sequence as that of the DNA fragment obtained and encoding the full length or a partial amino acid sequence of the protein of interest may be synthesized chemically.

The DNA thus obtained according to the invention encoding the full length or a partial amino acid sequence of a protein of interest is sometimes referred to as “the resultant DNA encoding the full length of a protein of interest” or the like.

For the production of a protein of interest, a DNA sequence obtained from a clone exhibiting a higher expression of a reporter gene than a clone retaining the wild-type DNA (e.g. cDNA or genomic DNA) sequence encoding the full length or a partial amino acid sequence of the protein of interest is used. Usually, a DNA sequence obtained from a clone exhibiting the highest expression of the reporter gene is used.

A DNA encoding the full-length amino acid sequence of the protein of interest, or a DNA having a DNA sequence encoding a partial amino acid sequence (e.g. N-terminal amino acid sequence) of the protein of interest and a DNA sequence downstream thereof encoding the remaining part of the protein of interest (e.g. DNA encoding an amino acid sequence spanning from the center to the C-terminal) is prepared. The DNA encoding the full length or a partial amino acid sequence of the protein of interest may be a DNA fragment obtained from the clone exhibiting the highest expression of the reporter gene or a chemically synthesized DNA fragment. The DNA encoding the remaining part of the protein of interest may be a cDNA, genomic DNA or synthesized DNA fragment. As long as the DNA fragment encodes the remaining part of the protein of interest, its nucleotide sequence may be different from the wild-type DNA sequence.

A DNA encoding the full length of the protein of interest can be prepared by ligating the above-obtained DNA fragment encoding a partial amino acid sequence of the protein of interest to the DNA fragment encoding the remaining part of the protein of interest. The ligation of these DNA fragment can be carried out according to known methods.

As a DNA suitable for industrial production of a protein of interest obtainable by the process of production of the present invention, the following examples may be given:
(1) a non-wild type DNA that comprises the same nucleotide sequence as that of the DNA obtained by the process of the invention encoding the full length or a partial amino acid sequence of the protein of interest contained in a transformant where high expression of the reporter gene has been confirmed, and encodes the full length of the protein of interest;
(2) a non-wild type DNA prepared by ligating to the 3′ end or/and 5′ end of the DNA obtained by the process of the invention encoding a partial amino acid sequence of the protein of interest contained in a transformant where high expression of the reporter gene has been confirmed a DNA/DNAs encoding the remaining amino acid sequence of the protein of interest.

A non-wild type DNA encoding the full length of a protein of interest means that this DNA does not comprise a sequence completely identical to the sequence of the naturally occurring DNA encoding the full length of the protein of interest.

More specifically, the following DNAs may be given as examples.
(1) In the case of rat GALP
   A DNA having the nucleotide sequence as shown in SEQ ID NO: 119.
(2) In the case of MPIF-1Δ23
   A DNA having the nucleotide sequence as shown in SEQ ID NO: 120.
(3) In the case of ZAQ-1 ligand
   A DNA having the nucleotide sequence as shown in SEQ ID NO: 121.
(4) In the case of rat GALP
   A DNA having the nucleotide sequence as shown in SEQ ID NO: 122.

A transformant capable of expressing the protein of interest can be obtained by integrating the resultant DNA encoding the full length of the protein of interest in a vector retaining a promoter functional in a host cell downstream of the promoter and then transforming a prokaryote with the resultant recombinant vector.

The protein of interest can be produced by culturing the resultant transformant.

Examples of vectors useful in the invention include plasmids derived from Escherichia coli (e.g. pBR322, pBR325, pUC12, and pUC13); plasmids derived from Bacillus subtilis (e.g. pUB110, pTP5 and pC194); plasmids derived from yeast (e.g. pSH19 and pSH15); bacteriophages such as λ-phage; animal viruses such as retrovirus, vaccinia virus, baculovirus; and other vectors such as pA1-11, pXT1, pRc/CMV,. pRc/RSV, pcDNAI/Neo and so on.

Any promoter may be used in the invention as long as it is appropriate for the host that will be used for expressing a gene of interest. When the host is an animal cell, examples of promoters useful in the invention include SR α promoter, SV40 promoter, LTR promoter, CMV promoter, HSV-TK promoter and so on.

Among these promoters, CMV promoter, SR α promoter or the like is preferably used. When the host is a bacteria belonging to the genus Escherichia, trp promoter, lac promoter, recA promoter, λ PL promoter, lpp promoter or the like is preferably used. When the host is a bacterium belonging to the genus Bacillus, SPO1 promoter, SPO2 promoter, penP promoter or the like is preferably used. When the host is a yeast, PHO5 promoter, PGK promoter, GAP promoter, ADH promoter, or the like is preferably used. When the host is an insect cell, polyhedrin promoter, P10 promoter or the like is preferably used.

The expression vectors may, if desired, further comprise enhancers, splicing signals, polyadenylation signals, selective markers, SV40 replication origin (hereinafter, sometimes abbreviated to “SV40 ori”) and the like. Examples of selective markers useful in the invention include dihydrofolate reductase (hereinafter, sometimes abbreviated to “dhfr”) gene [methotorexate (MD() resistance], ampicillin resistance gene (hereinafter, sometimes abbreviated to “Amp^{r}”), neomycin resistance gene (hereinafter, sometimes abbreviated to “Neo^{r}”: G418 resistance) and the like. In particular, when CHO (dhfr) cells are used in combination with dhfr gene as a selective marker, transformants with a gene of interest can be selected even in a thymidine-free medium.

A reporter gene can be inserted into the vector down stream of the promoter DNA according to known methods. For insertion of a DNA encoding the full length of a partial amino acid sequence of a protein of interest, usually, one or a plurality (preferably two or three) restriction enzyme recognition sites may be provided downstream of the promoter DNA and upstream of the reporter gene.

If desired, vectors may contain selective markers. Examples of selective markers useful in the invention include ampicillin resistance gene (Amp^{r}) and tetracycline resistance gene.

Transformants can be prepared by transforming a host with recombinant vectors into which a DNA encoding the full length or a partial amino acid sequence of the protein of interest has been inserted.

Examples of hosts useful for this purpose include bacteria belonging to the genus Escherichia, bacteria belonging to the genus Bacillus, yeasts, insect cells, insects, and animal cells.

Specific examples of bacteria belonging to the genus Escherichia useful in the invention include E. coli K12 DH1 [Proc. Natl. Acad. Sci. USA, Vol. 60, 160 (1968)], JM103 [Nucleic Acids Research, Vol. 9, 309 (1981)], JA221 [Journal of Molecular Biology, Vol. 2, 517-(1978)]), HB101 [Journal of Molecular Biology, Vol 41, 459 (1969)]and C600 [Genetics, Vol.39, 440 (1954)].

Specific examples of bacteria belonging to the genus Bacillus , useful in the invention include B. subtilis MU114 [Gene, Vol. 24, 255 (1983)] and 207-21 [Journal of Biochemistry, Vol.95, 87 (1984)].

Specific examples of yeasts useful in the invention include Saccharomyces cerevisiae AH22, AH22R⁻, NA87-11A, DKD-5D and 20B-12, Schizosaccharomyces pombe NCYC1913 and NCYC2036, and Pichia pastoris.

Specific examples of insect cells useful in the invention include, when the virus used is AcNPV, a cell line derived from larvae of Spodoptera frugiperda (Sf cells), MG1 cells derived from the midgut of Trichoplusia ni, High Five™ cells derived from eggs of Trichoplusia ni, Mamestra brassicae-derived cells and Estigmena acrea-derived cells. When the virus used is BmNPV, insect cells such as a silkworm-derived cell line (Bombyx mori N cells; BmN cells) may be used. Specific examples of Sf cells useful in the invention include Sf9 cells (ATCC CRL 1711) and Sf21 cells [both disclosed in Vaughn J. L. et al., In Vivo, 13, 213-217 (1977)].

Specific examples of insects useful in the invention include larvae of silkworm (Maeda et al., Nature, 315, 592 (1985)).

Specific examples of animal cells useful in the invention include a simian cell COS-7, Vero cells, a Chinese hamster cell CHO (hereinafter, abbreviated to “CHO cells”), a sdhfr gene-deficient Chinese hamster cell CHO (hereinafter, abbreviated to “CHO(dhfr) cells”), mouse L cells, mouse AtT-20 cells, mouse myeloma cells, rat GH3cells, and human FL cells.

Transformation of bactera belonging to the genus Escherichia can be performed in accordance with methods disclosed, for example, in Proc. Natl. Acad. Sci. USA, Vol. 69, 2110 (1972) and Gene, Vol. 17, 107 (1982). Transformation of bacteria belonging to the genus Bacillus can be performed in accordance with methods disclosed, for example, in Molecular & General Genetics, Vol. 168, 111 (1979).

Transformation of yeasts can be performed in accordance with methods disclosed, for example, in Methods in Enzymology, 194, 182-187(1991) and Proc. Natl. Acad. Sci. USA, Vo. 75, 1929 (1978).

Transformation of insect cells or insects can be performed in accordance with methods disclosed, for example, in Bio/Technology, 6,47-55 (1988).

Transformation of animal cells can be performed by methods disclosed, for example, in Cell Engineering, Separate Vol. 8, New Cell Engineering Experiment Protocol, 263-267(1995)(Shujunsha Co.) and Virology, Vol. 52, 456 (1973).

As a medium to culture transformants obtained from Escherichia or Bacillus bacteria as hosts, a liquid medium is appropriate. The medium is allowed to contain carbon sources, nitrogen sources, minerals, and so on which are necessary for the growth of the transformant. As carbon sources, glucose, dextrin, soluble starch, sucrose or the like may be enumerated. As nitrogen sources, organic or inorganic substances such as ammonium salts, nitrates, corn steep liquor, peptone, casein, meat extract, bean cake, potato extract, or the like may be enumerated. As minerals, calcium chloride, sodium dihydrogen phosphate, magnesium chloride, or the like may be enumerated. Further, yeast, vitamins, growth-promoting factors, etc. may also be added to the medium. Preferable pH of the medium is about 5-8.

As a medium to culture Escherichia bacteria, M9 medium containing glucose and casamino acid [Miller, Journal of Experiments in Molecular Genetics, 431-433, Cold Spring Harbor Laboratory, New York, (1972)] is preferable, for example. If necessary, drugs such as 3, β-indolyl acrylic acid can be added to the medium to improve the efficiency of the promoter. When the host is an Escherichia bacterium, the transformant is cultured usually at about 15-43° C. for about 3-24 hours. If necessary, aeration and stirring may be applied.

When the host is a Bacillus bacterium, the transformant is cultured usually at about 30-40° C. for about 6-24 hours. If necessary, aeration and stirring may also be applied.

As a medium to culture transformants obtained from yeasts as hosts, a medium such as Burkholder minimum medium [Bostian, K. L. et al., Proc. Natl. Acad. Sci. USA, Vol. 77, 4505 (1980)] or SD medium containing 0.5% casamino acid [Bitter, G. A. etal., Proc. Natl. Acad. Sci. USA, Vol. 81, 5330 (1984)] may be used, for example. It is preferable that the pH of the medium be adjusted to about 5-8. The transformant is cultured usually at about 20-35° C. for about 24-72 hours. If necessary, aeration and stirring may be applied.

As a medium to culture transformants obtained from insect cells or insects as hosts, Grace's Insect Medium [Grace, T. C. C., Nature, 195, 788 (1962)] supplemented with additives such as inactivated 10% bovine serum may be used, for example. It is preferable that the pH of the medium be adjusted to about 6.2-6.4. The transformant is cultured usually at about 27° C. for about 3-5 days. If necessary, aeration and stirring may be applied.

As a medium to culture transformants -obtained from animal cells as hosts, examples of useful media include MEM medium [Science, Vol. 122, 501 (1952)], DMEM medium [Virology, Vol. 8, 396 (1959)], RPMI 1640 medium [Journal of the American Medical Association, Vol. 199, 519 (1967)] and 199 medium [Proceedings of the Society of the Biological Medicine, Vol. 73, 1 (1950)] each containing about 5-20% fetal calf serum. Preferable pH of the medium is about 6 to about 8. The transformant is cultured usually at about 30-40° C. for about 15-60 hours. If necessary, aeration and stirring may be applied.

Separation and purification of the polypeptide from the resultant culture can be carried out, for example, according to the methods described below.

For extraction of the protein of interest from cultured microorganisms or cells, the microorganism cells are harvested by known methods after the cultivation, suspended in a suitable buffer, and disrupted by sonication or by lysozyme and/or freezing and thawing, etc.

Then, a crude extract of the polypeptide is obtained by centrifugation or filtration. The buffer may contain a protein denaturing agent such as urea or guanidine hydrochloride, or a surfactant such as Triton X-100™. If the protein of interest is secreted into the culture broth, the supernatant is separated from the microorganisms or cells after completion of the cultivation and collected by known methods.

Purification of the protein of interest contained in the thus obtained culture supernatant or extract can be performed by an appropriate combination of known methods for separation and purification. These known methods include methods utilizing solubility (such as salting out or sedimentation with solvents), methods mainly utilizing difference in molecular weight (such as dialysis, ultrafiltration, gel filtration and SDS-polyacrylamide gel electrophoresis), methods utilizing difference in electric charge (such as ion-exchange chromatography), methods utilizing specific affinity (such as affinity chromatography), methods utilizing difference in the hydrophobicity (such as reversed-phase high-performance liquid chromatography), and methods utilizing difference in isoelectric point (such as isoelectric electrophoresis).

When the thus obtained protein of interest is a free protein, the protein can be converted into the above-described salt by known methods or methods based thereon. On the contrary, when the protein of interest is obtained in a salt form, the salt can be converted into a free protein or another salt according to known methods or methods based thereon.

The protein of interest produced by the transformant can be arbitrarily modified or a part thereof can be removed therefrom by using an appropriate protein modification enzyme before or after the purification. Examples of such enzymes include trypsin, chymotrypsin, arginyl endopeptidase and protein kinase.

The extracted or separated/purified protein of interest is subjected to refolding, if necessary. Refolding can be performed by known methods described, for example, in Folding of Proteins, R. H. Pain (Ed.), 245-279 (1995) published by Springer-Verlag Tokyo or methods based thereon. Refolding can be performed, for example, by one-step or multi-step dilution in a buffer with no extraction agents (e.g. chaotropic solubilizers such as guanidine hydrochloride and urea, surfactants such as n-lauryl methyl glycine and SDS) or with extraction agents at low concentrations, dialysis with a semipermeable membrane, or replacement of a buffer using gel filtration. In order to prevent the aggregation of the protein of interest, arginine, polyethylene glycol, neutral surfactants, etc. may be added. For the formation of disulfide bonds in the protein, air oxidation, an oxidation-reduction buffer system, etc. may be employed. Examples of oxidation-reduction buffers include those based on glutathione, cysteine, dithiothreitol, 2-mercaptoethanol or cysteamine.

Further, the Met at the N-terminal of the protein of interest may be removed based on the method described in EP 0812856.

The resultant protein of interest may be used as a medicine, reagent, raw material, feed and so on depending on the nature of the protein.

When the protein of interest is used as a medicine, the protein may be used in a conventional manner. For example, the protein of interest may be used orally in the form of tablets (sugar-coated or enteric coated, if necessary), capsules, elixirs, microcapsules or the like; or parenterally in the form of injections such as aseptic solutions or suspensions in water or other pharmaceutically acceptable liquids. These preparations may be produced, for example, by mixing the compound or a salt thereof with physiologically acceptable carriers, flavoring agents, excipients, vehicles, antiseptics, stabilizers, binders, etc. in unit dosage forms. The amounts of active ingredients in these formulations are decided so that an appropriate dose within the specified range can be obtained.

Examples of additives which may be mixed in tablets, capsules, etc. include binders such as gelatin, corn starch, tragacanth gum and gum arabic, excipients such as crystalline cellulose, swelling agents such as corn starch, gelatin and alginic acid, lubricants such as magnesium stearate, sweetening agents such as sucrose, lactose and saccharin, and flavoring agents such as peppermint, akamono oil and cherry. When the unit dosage form is capsule, liquid carriers such as oils and fats may further be included in addition to the above-mentioned materials. Sterile compositions for injection can be formulated according to conventional practices in pharmaceutical manufacturing, e.g., by dissolving or suspending active ingredients in vehicles such as water for injection, naturally occurring vegetable oils such as sesame oil, coconut oil, etc.

Examples of aqueous liquids for injection include physiological saline and isotonic solutions containing glucose and other auxiliary agents (e.g. D-sorbitol, D-mannitol, sodium chloride, etc.). They may be used in combination with a suitable auxiliary solubilizer such as alcohol (e.g. ethanol), polyalcohol (e.g. propylene glycol, polyethylene glycol), nonionic surface active agent (e.g. Polysorbate 80™, HCO-50). Examples of oily liquids for injection include sesame oil, soybean oil, etc. They may be used in combination with an auxiliary solubilizer such as benzyl benzoate, benzyl alcohol, etc.

In addition, buffers (e.g. phosphate buffer, sodium acetate buffer, etc.), analgesic agents (e.g. benzalkonium chloride, procaine hydrochloride), stabilizers (e.g. human serum albumin, polyethylene glycol, etc.), preservatives (e.g. benzyl alcohol, phenol, etc.), antioxidants, etc. may also be admixed therewith. Usually, the prepared injections are filled in appropriate ampoules.

The thus obtained preparations may be administered to human or other manuals (e.g., mouse, rat, guinea pig, rabbit, sheep, pig, bovine, cat, dog, monkey, etc.).

In the specification and drawings of the present application, the abbreviations used for bases (nucleotides), amino acids and so forth are those recommended by the IUPAC-IUB Commission on Biochemical Nomenclature or those conventionally used in the art. Examples of such abbreviations are given below. Amino acids which may have optical isomers are intended to represent their L-isomer unless otherwise specified.
DNA: Deoxyribonucleic acid S
cDNA: Complementary deoxyribonucleic acid
A: Adenine
T: Thymine
G: Guanine
C: Cytosine
M: A or C
R: G or A
W: A or T
S: G or C
Y: T or C
K: G or T
V: A or G or C
H: A or C or T
D: A or G or T
B: G or C or T
N: A or C or G or T
RNA: Ribonucleic acid
mRNA: Messenger ribonucleic acid
dATP: Deoxyadenosine triphosphate
dTTP: Deoxythymidine triphosphate
dGTP: Deoxyguanosine triphosphate
dCTP: Deoxycytidine triphosphate.
ATP: Adenosine triphosphate
EDTA: Ethylenediaminetetracetic acid
SDS: Sodium dodecyl sulfate
Gly: Glycine
Ala: Alanine
Val: Valine
Leu: Leucine
Ile: Isoleucine
Ser: Serine
Thr: Threonine
Cys: Cysteine
Met: Methionine
Glu: Glutamic acid
Asp: Aspartic acid
Lys: Lysine
Arg: Arginine
His: Histidine
Phe: Phenylalanine
Tyr: Tyrosine
Trp: Tryptophan
Pro: Proline
Asn: Asparagine
Gln: Glutamine

The SEQ ID NOS of the SEQUENCE LISTING of the present specification represent the sequences as indicated below.

[SEQ ID NO: 1]

This shows the DNA sequence of primer 1.

[SEQ ID NO: 2]

This shows the DNA sequence of primer 2.

[SEQ ID NO: 3]

This shows the DNA sequence of synthetic DNA 1.

[SEQ ID NO: 4]

This shows the DNA sequence of synthetic DNA 2.

[SEQ ID NO: 5]

This shows the DNA sequence of synthetic DNA 3.

[SEQ ID NO: 6]

This shows the DNA sequence of synthetic DNA 4.

[SEQ ID NO: 7]

This shows the DNA sequence of synthetic DNA 5.

[SEQ ID NO: 8]

This shows the DNA sequence of synthetic DNA 6.

[SEQ ID NO: 9]

This shows the DNA sequence of primer 3.

[SEQ ID NO: 10]

This shows the DNA sequence of primer 4.

[SEQ ID NO: 11]

This shows the DNA sequence of primer 5.

[SEQ ID NO: 12]

This shows the DNA sequence of primer 6.

[SEQ ID NO: 13]

This shows the DNA sequence of primer 7.

[SEQ ID NO: 14]

This shows the DNA sequence of synthetic DNA 7.

[SEQ ID NO: 15]

This shows the DNA sequence of synthetic DNA 8.

[SEQ ID NO: 16]

This shows the DNA sequence of primer 8.

[SEQ ID NO: 17]

This shows the DNA sequence of synthetic DNA 9.

[SEQ ID NO: 18]

This shows the DNA sequence of synthetic DNA 10.

[SEQ ID NO: 19]

This shows the DNA sequence of synthetic DNA 11.

[SEQ ID NO: 20]

This shows the DNA sequence of synthetic DNA 12.

[SEQ ID NO: 21]

This shows the amino acid sequence of the N-terminal 15 amino acid residues of rat GALP.

[SEQ ID NO: 22]

This shows the nucleotide sequence of a DNA encoding the N-terminal 15 amino acid residues of rat GALP.

[SEQ ID NO: 23]

This shows the nucleotide sequence obtained from E. coli clone No. 1 obtained in Example 2.

[SEQ ID NO: 24]

This shows the nucleotide sequence obtained from E. coli clone No. 2 obtained in Example 2.

[SEQ ID NO: 25]

This shows the nucleotide sequence obtained from E. coli clone No. 3 obtained in Example 2.

[SEQ ID NO: 26]

This shows the nucleotide sequence obtained from E. coli clone No. 4 obtained in Example 2.

[SEQ ID NO: 27]

This shows the nucleotide sequence obtained from E. coli clone No. 5 obtained in Example 2.

[SEQ ID NO: 28]

This shows the nucleotide sequence obtained from E. coli clone No. 6 obtained in Example 2.

[SEQ ID NO: 29]

This shows the nucleotide sequence obtained from E. coli clone No. 7 obtained in Example 2.

[SEQ ID NO: 30]

This shows the nucleotide sequence obtained from E. coli clone No. 8 obtained in Example 2.

[SEQ ID NO: 31]

This shows the nucleotide sequence obtained from E. coli clone No. 9 obtained in Example 2.

[SEQ ID NO: 32]

This shows the nucleotide sequence obtained from E. coli clone No. 10 obtained in Example:2.

[SEQ ID NO: 33]

This shows the nucleotide sequence obtained from E. coli clone No. 11 obtained in Example 2.

[SEQ ID NO: 34]

This shows the nucleotide sequence obtained from E. coli clone No. 12 obtained in Example 2.

[SEQ ID NO: 35]

This shows the nucleotide sequence obtained from E. coli clone No. 13 obtained in Example 2.

[SEQ ID NO: 36]

This shows the nucleotide sequence obtained from E. coli clone No. 14 obtained in Example 2.

[SEQ ID NO: 37]

This shows the nucleotide sequence obtained from E. coli clone No. 15 obtained in Example 2.

[SEQ ID NO: 38]

This shows the nucleotide sequence obtained from E. coli clone No. 16 obtained in Example 2.

[SEQ ID NO: 39]

This shows the nucleotide sequence obtained from E. coli clone No. 17 obtained in Example 2.

[SEQ ID NO: 40]

This shows the nucleotide sequence obtained from E. coli clone No. 18 obtained in Example 2.

[SEQ ID NO: 41]

This shows the amino acid sequence of the N-terminal 5 amino acid residues of MPIF 1Δ23.

[SEQ ID NO: 42]

This shows the nucleotide sequence of a DNA encoding the N-terminal 5 amino acid sequence of MPIF 1Δ23.

[SEQ ID NO: 43]

This shows the nucleotide sequence obtained from E. coli clone No. 1 obtained in Example 5.

[SEQ ID NO: 44]

This shows the nucleotide sequence obtained from E. coli clone No. 2 obtained in Example 5.

[SEQ ID NO: 45]

This shows the nucleotide sequence obtained from E. coli clone No. 3 obtained in Example 5.

[SEQ ID NO: 46]

This shows the nucleotide sequence obtained from E. coli clone No. 4 obtained in Example 5.

[SEQ ID NO: 47]

This shows the nucleotide sequence obtained from E. coli clone No. 5 obtained in Example 5.

[SEQ ID NO: 48]

This shows the nucleotide sequence obtained from E. coli clone No. 6 obtained in Example 5.

[SEQ ID NO: 49]

This shows the nucleotide sequence obtained from E. coli clone No. 7 obtained in Example 5.

[SEQ ID NO: 50]

This shows the nucleotide sequence obtained from E. coli clone No. 8 obtained in Example 5.

[SEQ ID NO: 51]

This shows the nucleotide sequence obtained from E. coli clone No. 9 obtained in Example 5.

[SEQ ID NO: 52]

This shows the nucleotide sequence obtained from E. coli clone No. 10 obtained in Example 5.

[SEQ ID NO: 53]

This shows the nucleotide sequence obtained from E. coli clone No. 11 obtained in Example 5.

[SEQ ID NO: 54]

This shows the nucleotide sequence obtained from E. coli clone No. 12. obtained in Example 5.

[SEQ ID NO: 55]

This shows the nucleotide sequence obtained from E. coli clone No. 13 obtained in Example 5.

[SEQ ID NO: 56]

This shows the nucleotide sequence obtained from E. coli clone No. 14 obtained in Example 5.

[SEQ ID NO: 57]

This shows the nucleotide sequence obtained from E. coli clone No. 15 obtained in Example 5.

[SEQ ID NO: 58]

This shows the nucleotide sequence obtained from E. coli clone No. 16 obtained in Example 5.

[SEQ ID NO: 59]

This shows the nucleotide sequence obtained from E. coli clone No. 17 obtained in Example 5.

[SEQ ID NO: 60]

This shows the nucleotide sequence obtained from E. coli clone No. 18 obtained in Example 5.

[SEQ ID NO: 61]

This shows the nucleotide sequence obtained from E. coli clone No. 19 obtained in Example 5.

[SEQ ID NO: 62]

This shows the nucleotide sequence obtained from E. coli clone No. 20 obtained in Example 5.

[SEQ ID NO: 63]

This shows the nucleotide sequence obtained from E. coli clone No. 21 obtained in Example 5.

[SEQ ID NO: 64]

This shows the nucleotide sequence obtained from E. coli clone No. 22 obtained in Example 5.

[SEQ ID NO: 65]

This shows the nucleotide sequence obtained from E. coli clone No. 23 obtained in Example 5.

[SEQ ID NO: 66]

This shows the nucleotide sequence obtained from E. coli clone No. 24 obtained in Example 5.

[SEQ ID NO: 67]

This shows the nucleotide sequence obtained from E. coli clone No. 25 obtained in Example5.

[SEQ ID NO: 68]

This shows the nucleotide sequence obtained from E. coli clone No. 26 obtained in Example 5.

[SEQ ID NO: 69]

This shows the nucleotide sequence obtained from E. coli clone No. 27 obtained in Example 5.

[SEQ ID NO: 70]

This shows die nucleotide sequence obtained from E. coli clone No. 28 obtained in Example 5.

[SEQ ID NO: 71]

This shows the nucleotide sequence obtained from E. coli clone No. 29 obtained in Example 5.

[SEQ ID NO: 72]

This shows the nucleotide sequence obtained from E. coli clone No. 30 obtained in Example 5.

[SEQ ID NO: 73]

This shows the nucleotide sequence obtained from E. coli clone No. 31 obtained in Example 5.

[SEQ ID NO: 74]

This shows the nucleotide sequence obtained from E. coli clone No. 32 obtained in Example 5.

[SEQ ID NO: 75]

This shows the nucleotide sequence of synthetic DNA 13.

[SEQ ID NO: 76]

This shows the nucleotide sequence of synthetic DNA 14.

[SEQ ID NO: 77]

This shows the nucleotide sequence of synthetic DNA 15.

[SEQ ID NO: 78]

This shows the nucleotide sequence of synthetic DNA 16.

[SEQ ID NO: 79]

This shows the nucleotide sequence of synthetic DNA 17.

[SEQ ID NO: 80]

This shows the nucleotide sequence of synthetic DNA 18.

[SEQ ID NO: 81]

This shows the nucleotide sequence of synthetic DNA 19.

[SEQ ID NO: 82]

This shows the nucleotide sequence of synthetic DNA 20.

[SEQ ID NO: 83]

This shows the nucleotide sequence of synthetic DNA 21.

[SEQ ID NO: 84]

This shows the nucleotide sequence of synthetic DNA 22.

[SEQ ID NO: 85]

This shows the nucleotide sequence of synthetic DNA 23.

[SEQ ID NO: 86]

This shows the nucleotide sequence of synthetic DNA 24.

[SEQ ID NO: 87]

This shows the nucleotide sequence of synthetic DNA 25.

[SEQ ID NO: 88]

This shows the nucleotide sequence of synthetic DNA 26.

[SEQ ID NO: 89]

This shows the nucleotide sequence of synthetic DNA 27.

[SEQ ID NO: 90]

This shows the nucleotide sequence of synthetic DNA 28.

[SEQ ID NO: 91]

This shows the nucleotide sequence of synthetic DNA 29.

[SEQ ID NO: 92]

This shows the nucleotide sequence of synthetic DNA 30.

[SEQ ID NO: 93]

This shows the nucleotide sequence of synthetic DNA 31.

[SEQ ID NO: 94]

This shows the nucleotide sequence of synthetic DNA 32.

[SEQ ID NO: 95]

This shows the nucleotide sequence of synthetic DNA 33.

[SEQ ID NO: 96]

This shows the nucleotide sequence of synthetic DNA 34.

[SEQ ID NO: 97]

This shows the nucleotide sequence of synthetic DNA 35.

[SEQ ID NO: 98]

This shows the nucleotide sequence of synthetic DNA 36.

[SEQ ID NO. 99]

This shows the nucleotide sequence of synthetic DNA 37.

[SEQ ID NO. 100]

This shows the nucleotide sequence of synthetic DNA 38.

[SEQ ID NO: 101]

This shows the nucleotide sequence of synthetic DNA 39.

[SEQ ID NO: 102]

This shows the nucleotide sequence of synthetic DNA 40.

[SEQ ID NO: 103]

This shows the nucleotide sequence of synthetic DNA 41.

[SEQ ID NO: 104]

This shows the nucleotide sequence of synthetic DNA 42.

[SEQ ID NO: 105]

This shows the nucleotide sequence of synthetic DNA 43.

[SEQ ID NO: 106]

This shows the nucleotide sequence of synthetic DNA 44.

[SEQ ID NO: 107]

This shows the nucleotide sequence of synthetic DNA 45.

[SEQ ID NO: 108]

This shows the nucleotide sequence of synthetic DNA 46.

[SEQ ID NO: 109]

This shows the nucleotide sequence of synthetic DNA 47.

[SEQ ID NO: 110]

This shows the nucleotide sequence of synthetic DNA 48.

[SEQ ID NO: 111]

This shows the nucleotide sequence of synthetic DNA 49.

[SEQ ID NO: 112]

This shows the nucleotide sequence of synthetic DNA 50.

[SEQ ID NO: 113]

This shows the nucleotide sequence of synthetic DNA 51.

[SEQ ID NO: 114]

This shows the nucleotide sequence of synthetic DNA 52.

[SEQ ID NO: 115]

This shows the nucleotide sequence of synthetic DNA 53.

[SEQ ID NO: 116]

This shows the amino acid sequence of a GPR8 ligand.

[SEQ ID NO: 117]

This shows the amino acid sequence of a ZAQ-1 ligand.

[SEQ ID NO: 118]

This shows the amino acid sequence of a bFGF CS23 mutein.

[SEQ ID NO: 119]

This shows the nucleotide sequence of a DNA encoding a rat GALP obtained by the production process of the invention.

[SEQ ID NO: 120]

This shows the nucleotide sequence of a DNA encoding an MPIF-1Δ23 obtained by the production process of the invention.

[SEQ ID NO: 121]

This shows the nucleotide sequence of a DNA encoding a ZAQ-1 ligand obtained by the production process of the invention.

[SEQ ID NO: 122]

This shows the nucleotide sequence of a DNA encoding a rat GALP obtained by the production process of the invention.

Escherichia coli (MM294 (DE3), pTCHGH—Na), which is a transformant carrying plasmid pTCHGH—Na that is used in Example 1 described later, has been deposited with the International Patent Organism Depository, National Institute of Advanced Industrial Science and Technology (former designation: National Institute of Bioscience and Human Technology, Agency of Industrial Science and Technology, Ministry of International Trade and Industry (NIBH)) located at Central 6, 1-1 Higashi 1-chome, Tsukuba City, Ibaraki Pref. since Dec. 10, 1997 under the Accession No. FERM BP-6888, and with the Institute for Fermentation, Osaka (IFO) located at 17-85 Jusanboncho 2-chome, Yodogawa-ku, Osaka City, Osaka Pref. since Oct. 16, 1997 under the Accession No. IFO 16124.

Escherichia coli JM109/pGFPNP, a transformant obtained in Example 1 described later, has been deposited with the International Patent Organism Depository, National Institute of Advanced Industrial Science and Technology (former designation: National Institute of Bioscience and Human Technology, Agency of Industrial Science and Technology, Ministry of International Trade and Industry (NIBH)) since Jul. 17, 2000 under the Accession No. FERM BP-7223, and with the Institute for Fermentation, Osaka (IFO) since Apr. 24, 2000 under the Accession No. IFO 16426.

Escherichia coli MM294 (DE3)/pTFCRGAL, a transformant obtained in Example 3 described later, has been deposited with the International Patent Organism Depository, National Institute of Advanced Industrial Science and Technology (former designation: National Institute of Bioscience and Human Technology, Agency of Industrial Science and Technology, Ministry of International Trade and Industry (NIBH)) since Jul. 17, 2000 under the Accession No. FERM BP-7226, and with the Institute for Fermentation, Osaka (IFO) since Apr. 24, 2000 under the Accession No. IFO 16429.

Escherichia coli MM294 (DE3)/pTFCS4, a transformant obtained in Example 4 described later, has been deposited with the International Patent Organism Depository, National Institute of Advanced Industrial Science and Technology (former designation: National Institute of Bioscience and Human Technology, Agency of Industrial Science and Technology, Ministry of International Trade and Industry (NIBH)) since Jul. 17, 2000 under the Accession No. FERM BP-7227, and with the Institute for Fermentation, Osaka (IFO) since Apr. 24, 2000 under the Accession No. IFO 16430.

Escherichia coli MM294 (DE3)/pTClld23-MPIF1, which is a transformant carrying plasmid pTCII/d23-MPIF1 that is used in Example 8 described later, has been deposited with the International Patent Organism Depository, National Institute of Advanced Industrial Science and Technology (former designation: National Institute of Bioscience and Human Technology, Agency of Industrial Science and Technology, Ministry of International Trade and Industry (NIBH)) since Nov. 24, 1998 under the Accession No. FERM BP-6582, and with the Institute for Fermentation, Osaka (IFO) since Oct. 27, 1998 under the Accession No. IFO 16212.

Escherichia coli JM 109/pNPMPIF, a transformant obtained in Example 8 described later, has been deposited with the International Patent Organism Depository, National Institute of Advanced Industrial Science and Technology (former designation: National Institute of Bioscience and Human Technology, Agency of Industrial Science and Technology, Ministry of International Trade and Industry (NIBH)) since Jul. 17, 2000 under the Accession No. FERM BP-7224, and with the Institute for Fermentation, Osaka (IFO) since Apr. 24, 2000 under the Accession No. IFO 16427.

Escherichia coli JM109/pNPMPIFNO, a transformant obtained in Example 9 described later, has been deposited with the International Patent Organism Depository, National Institute of Advanced Industrial Science and Technology (former designation: National Institute of Bioscience and Human Technology, Agency of Industrial Science and Technology, Ministry of International Trade and Industry (NIBH)) since Jul. 17, 2000 under the Accession No. FERM BP-7225, and with the Institute for Fermentation, Osaka (IFO) since Apr. 24, 2000 under the Accession No. IFO 16428.

Escherichia coli TOP10/pHMITG, which is a transformant carrying plasmid pHMITG that is used in Example 12 described later, has been deposited with the International Patent Organism Depository, National Institute of Advanced Industrial Science and Technology (former designation: National Institute of Bioscience and Human Technology, Agency of Industrial Science and Technology, Ministry of International Trade and Industry (NIBH)) since Jul. 13, 2000 under the Accession No. FERM BP-7220, and with the Institute for Fermentation, Osaka (IFO) since May 26, 2000 under the Accession No. IFO 16441.

Escherichia coli MM294 (DE3)/pTCIIZAQN, a transformant obtained in Example 12 described later, has been deposited with the International Patent Organism Depository, National Institute of Advanced Industrial Science and Technology since May 24, 2001 under the Accession No. FERM BP-7608, and with the Institute for Fermentation, Osaka (IFO) since May 15, 2001 under the Accession No. IFO 16627.

Escherichia coli MM294 (DE3)/pTCIIZAQS, a transformant obtained in Example 13 described later, has been deposited with the International Patent Organism Depository, National Institute of Advanced Industrial Science and Technology since May 24, 2001 under the Accession No. FERM BP-7609, and with the Institute for Fermentation, Osaka (IFO) since May 15, 2001 under the Accession No. IFO 16628.

Escherichia coli MM294 (DE3)/pTCIIZAQNO, a transformant obtained in Example 15 described later, has been deposited with the International Patent Organism Depository, National Institute of Advanced Industrial Science and Technology since May 24, 2001 under the Accession No. FERM BP-7610, and with the Institute for Fermentation, Osaka (IFO) since May 15, 2001 under the Accession No. IFO 16629.

Escherichia coli MM294 (DE3)/pTCRGDE, a transformant obtained in Example 17 described later, has been deposited with the International Patent Organism Depository, National Institute of Advanced Industrial Science and Technology since May 24, 2001 under the Accession No. FERM BP-7612, and with the Institute for Fermentation, Osaka (IFO) since May 17, 2001 under the Accession No. IFO 16631.

Escherichia coli MM294 (DE3)/pTCRGNODE, a transformant obtained in Example 18 described later, has been deposited with the International Patent Organism Depository, National Institute of Advanced Industrial Science and Technology since May 24, 2001 under the Accession No. FERM BP-7614, and with the Institute for Fermentation, Osaka (IFO) since May 17, 2001 under the Accession No. IFO 16633.

Escherichia coli MM294 (DE3)/pTCRGTODE, a transformant obtained in Example 20 described later, has been deposited with the International Patent Organism Depository, National Institute Advanced Industrial Science and Technology since May 24, 2001 under the Accession No. FERM BP-7613, and with the Institute for Fermentation, Osaka (IFO) since May 17, 2001 under the Accession No. IFO 16632.

### EXAMPLES

Hereinbelow, the present invention will be described in more detail with reference to the following Examples. However, these Examples are not intended to limit the scope of the present invention. Gene manipulations using E. coli were performed in accordance with those methods described in the book “Molecular Cloning”.

### Example 1

Construction of Plasmids for Searching for Optimized Sequences

1) Construction of Plasmid pGFPuvqc

In order to eliminate the NdeI restriction site located in the GFPuv structural gene in pGFPuv (Clontech), the codon of the His at position 77 was changed from CAT to CAC through site-directed mutagenesis (QuickChange™ Site Directed Mutagenesis Kit, Stratagene) using primer 1: CGTTATCCGGATCACATGAAACGGCATG (SEQ ID NO: 1) and primer 2: CATGCCGTTTCATGTGATCCGGATAACG (SEQ ID NO: 2). Thus, plasmid pGFPuvqc was obtained.

2) Construction of Plasmid pGFPuvqd

Plasmid pGFPuvqc was digested with HindIII (Takara Shuzo) and KpnI (Takara Shuzo), followed by agarose gel electrophoresis to cut out an approx. 3.3 kbp band. The DNA was recovered from the gel using QIAquick Gel Extraction Kit (Qiagen). Synthetic DNA 1: AGCTTCATATGTTCGAAGTACTAGATCTGGTAC (SEQ ID NO: 3) and synthetic DNA 2: CAGATCTAGTACTTCGAACATATGA (SEQ ID NO: 4) (100 pmol each) were dissolved in Th buffer, retained at 90° C. for 10 min, and then gradually cooled to room temperature to perform annealing. The thus annealed DNA fragment was inserted into pGFPuvqc between the HindIII and KpnI sites using Ligation kit ver. 2 (Takara Shuzo) to thereby obtain plasmid pGFPuvqd.

3) Construction of Plasmid pGFPNP

Plasmid pNPHGH in which the T7 promoter of pTCHGH—Na described in Reference Example 1 in WO 00/20439 is replaced with a synthetic promoter NP2 was constructed as described below. Briefly, plasmid pTCHGH—Na was digested with EcoRI (Takara Shuzo) and XbaI (Takara Shuzo), followed by agarose gel electrophoresis to cut out an approx. 4.6 kbp band. The DNA was recovered from the gel using QIAquick Gel Extraction Kit (Qiagen). One hundred pmol each of synthetic DNA 3: AATTCTATAAAAATAATTGTTGACATATITATAAATITMGGCATAATAGATCTAATTGTGAGCGGATAACAATTCTGCAGAAGCCTGAGCTCGGTACCCGGGGATCCT (SEQ ID NO: 5) comprising the synthetic promoter NP2, and synthetic DNA 4: CTAGAGGATCCCCGGGTACCGAGCTCAAGCTTCTGCAGAATTGTTATCCGCTCACAATTAGATCTATTATGCCAAAATTTATAAAATATGTCAACAATTATTTTTTATAG (SEQ ID NO: 6) comprising a nucleotide sequence complementary to the synthetic promoter NP2 were dissolved in TE buffer, retained at 90° C. for 10 min, and then gradually cooled to room temperature to perform annealing. The resultant double-stranded DNA fragment was inserted into pTCHGH—Na between the EcoRI and XbaI sites using Ligation kit ver. 2 (Takara Shuzo) to thereby obtain plasmid pNPHGH.

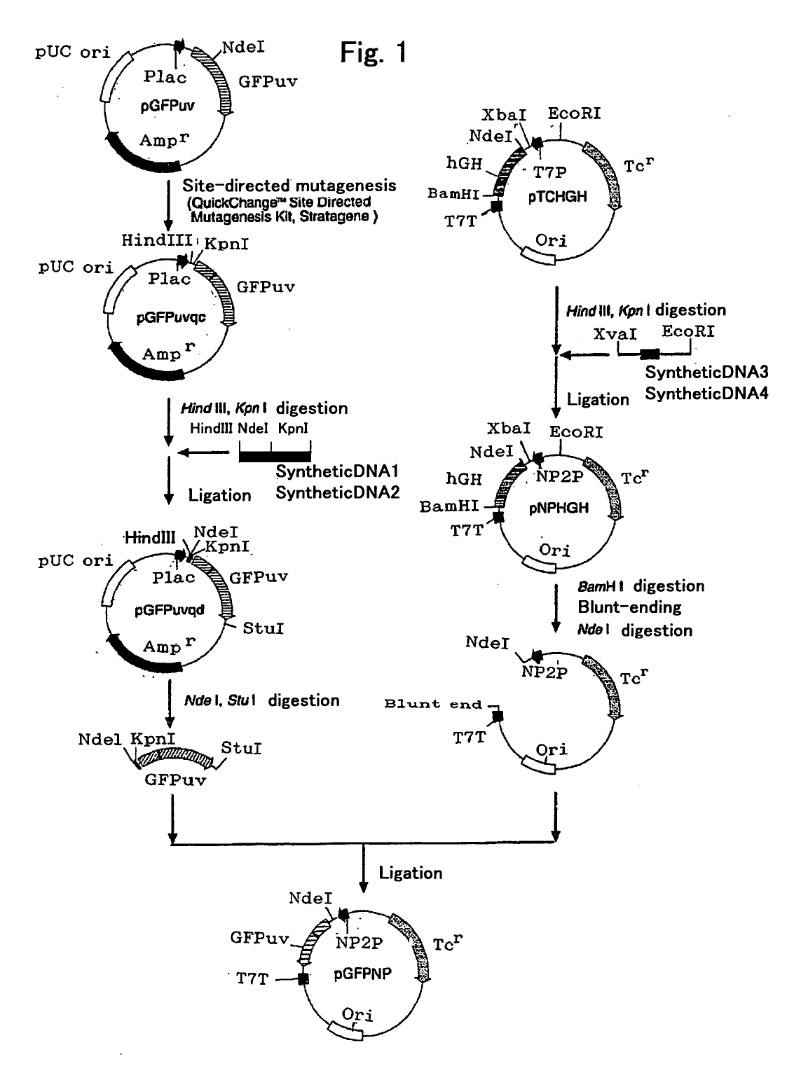

Plasmid pNPHGH was digested with BamHI (Takara Shuzo), followed by blunt-ending with DNA Blunting Kit (Takara Shuzo). After further digestion with NdeI the digest was subjected to agarose gel electrophoresis to cut out an approx. 4.6 kbp band. The DNA was recovered from the gel using QIAquick Gel Extraction Kit (Qiagen). Plasmid pGFPuvqd was digested with NdeI and StuI followed by agarose gel electrophoresis to cut out an approx. 0.8 kbp band. A DNA comprising the GFPuv structural gene was recovered from the gel using QIAquick Gel Extraction Kit (Qiagen). The thus recovered DNA was inserted into pNPHGH between the NdeI site and BamHI (blunted) site using Ligation kit ver. 2 (Takara Shuzo) to thereby obtain plasmid pGFPNP for use in selecting N-terminal optimized sequences (FIG. 1).

Plasmid pGFPNP is an expression plasmid containing a GFPuv structural gene to be transcribed from NP2 promoter and having NdeI, ScaI and AgeI restriction sites upstream of the GFPuv structural gene. It is possible to insert DNA fragments into this plasmid using these restriction enzyme sites.

E. coli JM109 was transformed with plasmid pGFPNP to thereby obtain E. coli JM109/pGFPNP.

### Example 2

Search for Rat GALP Sequences Optimized for Expression

Plasmid pGFPNP was digested with NdeI (Takara Shuzo) and ScaI (Takara Shuzo), followed by agarose gel electrophoresis to cut out an approx. 4.9 kbp band. The DNA was recovered from the gel using QIAquick Gel Extraction Kit (Qiagen).

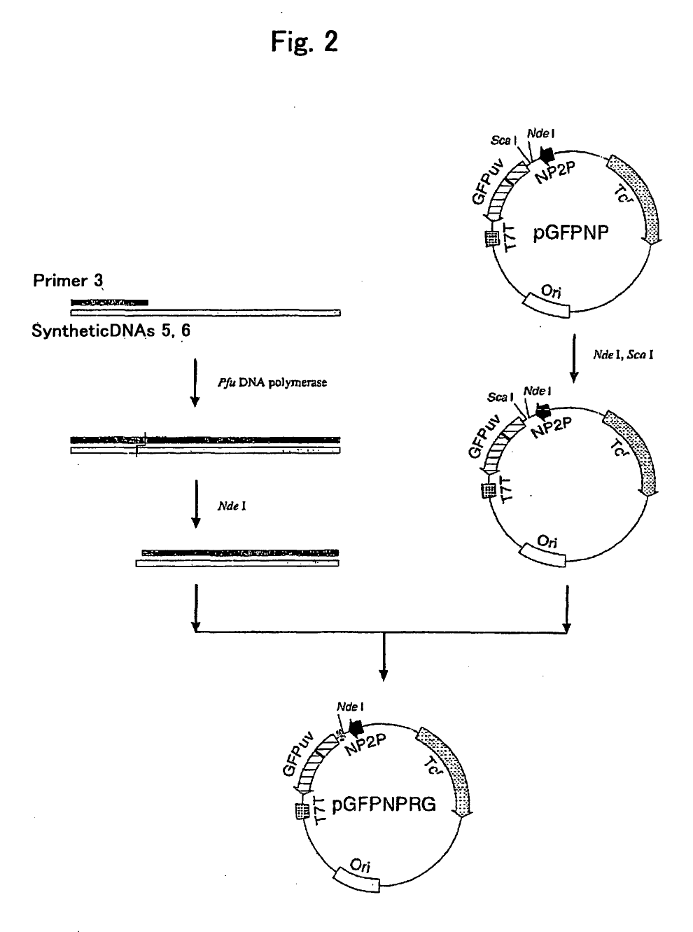

Random nucleotide sequences encoding N-terminal amino acids of rat GALP (a rat type ligand peptide for rat galanin receptor (1-60); WO 99/48920) were prepared as described below. Briefly, double-stranded DNA fragments were prepared with Pfu DNA polymerase (Stratagene) using mixed bases-containing synthetic DNA 5: AGGTAACCAGCACTATTTAAAGTCCANCCNCCNCGNCCNCGRTGNGCNGGNGCCATATGTATATCTCCTTCTTAAAG (SEQ ID NO: 7) and synthetic DNA 6: AGGTAACCAGCACTATTTAAAGTCCANCCNCCYCTNCCYCTRTGNGCNGGNGCCATATGTATATCTCCTTCTTAAAG (SEQ ID NO: 8) as templates and primer 3: CTTTAAGAAGGAGATATACATATG (SEQ ID NO: 9) as a primer. The extension reaction solution was composed of 5 μl of Pfu DNA polymerase, 5 μl each of 10× Reaction buffer attached to the polymerase and dNTP mixture (Takara Shuzo), 1 μl each of synthetic DNA 3 (42.2 μmol/L), synthetic DNA 4 (5 μmol/L) and primer 3 (100 μmol/L), and 32 μl of distilled water. The extension reaction was performed at 94° C. for 30 sec, at 58° C. for 30 sec, and at 72° C. for 20 min. The resultant reaction solution was concentrated and desalted with Microcon 30 (Nihon Millipore), followed by digestion of resultant double-stranded DNA fragments with NdeI (Takara Shuzo). This digestion with NdeI was conducted as described below. Briefly, a reaction solution consisting of 10 μl of the above concentrated/desalted reaction solution, 2 μl of Buffer H (Takara Shuzo), 7 μl of distilled water and 1 μl of NdeI was retained at 37° C. for 3 hr to perform digestion. From the reaction solution after this NdeI digestion, DNA fragments were purified using QIAquick Gel Extraction Kit (Qiagen). These DNA fragments were inserted individually into pGFPNP2 between the NdeI and ScaI sites using Ligation kit ver. 2 (Takara Shuzo) to thereby prepare a set of expression plasmid pGFPNPRG having at the N-terminal of GFPuv a random nucleotide sequence encoding N-terminal amino acids of rat GALP (FIG. 2).

E. coli JM109 was transformed with this set of expression plasmid pGFPNPRG, coated on LB agar medium containing 10 mg/L tetracycline, and cultured overnight at 37° C. for colony formation. The thus formed colonies were observed under a UV ramp at 354 nm, followed by selection of 2 colonies emitting intense fluorescence and 16 colonies emitting weak fluorescence. These colonies were subjected to colony PCR using primer 4: TTCATACACGGTGCCTGACTGCGTTAG (SEQ ID NO: 10) and primer 5: TCAACAAGAATTGGGACAACTCC (SEQ ID NO: 11), followed by agarose gel electrophoresis to confirm that a DNA fragment of interest is inserted. The products of this colony PCR were purified with QIAquick PCR Purification Kit (Qiagen), and their nucleotide sequences were analyzed with primers 4 and 5. As a result, the nucleotide sequences shown in Table 1 were obtained. Of these, the nucleotide sequences No. 1 and No. 2 were obtained from those E. coli clones emitting intense fluorescence.

TABLE 1

| Wild | Residue | 1 | 2 | 3 | 4 | 5 | 6 | 7 | 8 | 9 | 10 | 11 | 12 | 13 | 14 |
|---|---|---|---|---|---|---|---|---|---|---|---|---|---|---|---|
| type | Amino acid | Ala | Pro | Ala | His | Arg | Gly | Arg | Gly | Gly | Trp | Thr | Leu | Asn | Ser |
| sequence | Nucleotide | GCA | CCT | GCT | CAC | AGG | GGA | CGA | GGA | GGC | TGG | ACC | CTC | AAT | AGT |
| Nucleotide sequences | No. 1 | GCT | CCA | GCG | CAT | CGT | GGG | CGT | GGT | GGT | TGG | ACT | TTA | AAT | AGT |
| obtained from | No. 2 | GCA | CCT | GCA | CAT | CGT | GGG | CGA | GGA | GGC | TGG | ACT | TTA | AAT | AGT |
| E. coli clones | No. 3 | GCA | CCA | CAC | GCA | CGC | GGA | CGA | GGG | GGA | TGG | ACT | TTA | AAT | AGT |
| | No. 4 | GCA | CCA | GCA | CAC | AGA | GGA | AGG | GGC | GGA | TGG | ACT | TTA | AAT | AGT |
| | No. 5 | GCA | CCA | GCT | CAC | CGA | GGC | CGA | GGA | GGC | TGG | ACT | TTA | AAT | AGT |
| | No. 6 | GCA | CCC | GCT | CAC | CGC | GGA | CGT | GGA | GGC | TGG | ACT | TTA | AAT | AGT |
| | No. 7 | GCA | CCA | GCA | CAT | AGA | GGA | AGA | GGA | GGC | TGG | ACT | TTA | AAT | AGT |
| | No. 8 | GCA | CCG | GCA | CAC | CGC | GGA | CGA | GGG | GGT | TGG | ACT | TTA | AAT | AGT |
| | No. 9 | GCA | CCG | GCG | CAC | CGC | GGA | CGT | GGC | AGA | TGG | ACT | TTA | AAT | AGT |
| | No. 10 | GCC | CCA | GCA | CAT | CGC | GGA | CGT | GGC | GGA | TGG | ACT | TTA | AAT | AGT |
| | No. 11 | GCC | CCA | GCA | CAC | CGC | GGT | CGC | GGC | GGT | TGG | ACT | TTA | AAT | AGT |
| | No. 12 | GCT | CCA | GCA | CAC | CGG | GGG | CGC | GGG | GGA | TGG | ACT | TTA | AAT | AGT |
| | No. 13 | GCA | CCG | GCA | CAC | CGC | GGC | CGC | GGA | GGA | TGG | ACT | TTA | AAT | AGT |
| | No. 14 | GCG | CCA | GCT | CAT | CGG | GGT | CGT | GGC | GGG | TGG | ACT | TTA | AAT | AGT |
| | No. 15 | GCT | CCA | GCT | CAT | CGA | GGA | CGC | GGC | GGT | TGG | ACT | TTA | AAT | AGT |
| | No. 16 | GCG | CCA | GCA | CAC | CGT | GGC | CGA | GGC | GGC | TGG | ACT | TTA | AAT | AGT |
| | No. 17 | GCA | CCG | GCG | CAC | CGC | GGA | CGT | GGC | AGA | TGG | ACT | TTA | AAT | AGT |
| | No. 18 | GCC | CCA | GCG | CAT | CGG | GGG | CGT | GGC | GGC | TGG | ACT | TTA | AAT | AGT |

### Example 3

Construction of Clones that Express Rat GALP Encoded by Wild Type Nucleotide Sequence

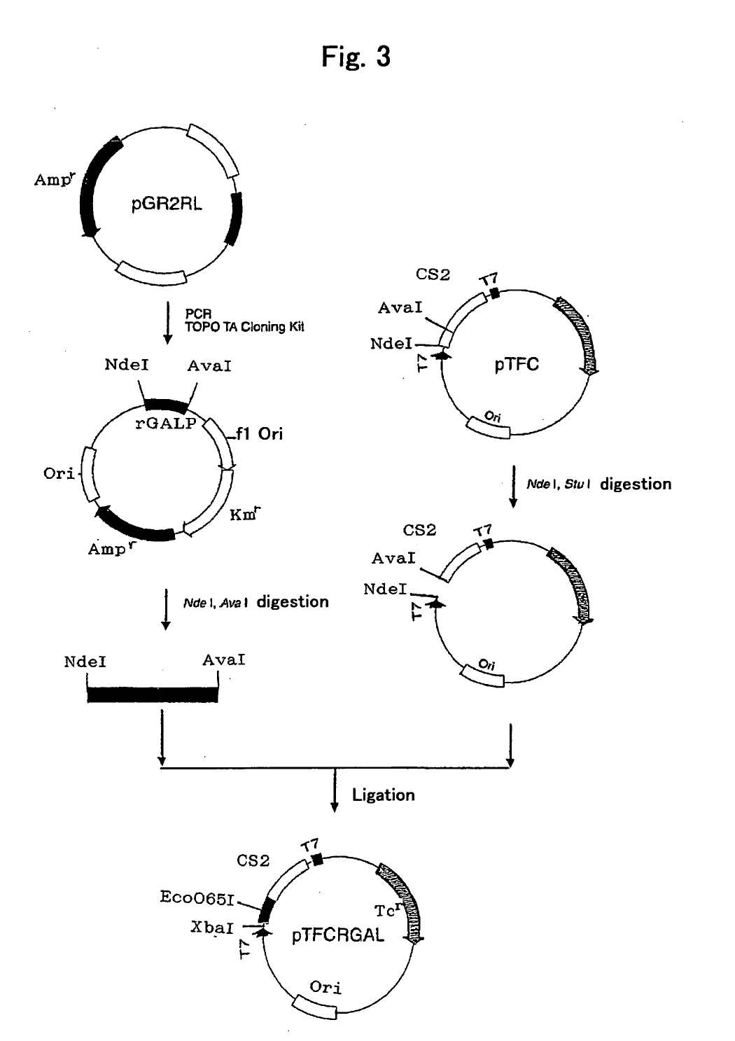

As a rat GALP expression plasmid, plasmid pTFCRGAL was constructed as described below for expression of rat GSLP-CS23 fusion protein from T7 promoter (FIG. 3).

Plasmid pTFC described in Example 21 in WO 99/48920 was digested with NdeI and AvaI, followed by agarose gel electrophoresis to cut out an approx. 3.9 kbp band. The DNA was recovered from the gel using QIAquick Gel Extraction Kit (Qiagen).

A fragment containing a rat GALR2L structural gene was amplified by PCR from plasmid pGR2RL4 (described in Example 18 in WO 99/48920) where a rat GALP structural gene is cloned, using primer 6: AGCATATGGCACCTGCTCACAGG (SEQ ID NO: 12) having an initiation codon ATG and an NdeI restriction site upstream of rat GALR2L structural gene and primer 7: CCCTCGGGGCAGGTCATCCTTGGAGAG (SEQ ID NO: 13) having a TGC codon encoding Cys and an AvaI restriction site downstream of the GALP structural gene. The resultant fragment was cloned using TOPO TA Cloning Kit to thereby obtain plasmid pCR2.1TOPO/rGAL. This plasmid pCR2.1TOPO/rGAL was digested with NdeI and AvaI, followed by agarose gel electrophoresis to cut out an approx. 0.2 kbp band. The DNA was recovered from the gel using QIAquick Gel Extraction Kit (Qiagen). This DNA fragment was ligated to pTFC between the NdeI and AvaI sites using Ligation kit ver. 2 (Takara Shuzo) to thereby obtain plasmid pTFCRGAL. This plasmid comprises a DNA (SEQ ID NO: 119) comprising the nucleotide sequence No. 1 described in Example 2 and having a nucleotide sequence encoding rat GALP.

E. coli MM294 (DE3) was transformed with this expression plasmid pTFCRGAL to thereby obtain E. coli MM294 (DE3)/pTFCRGAL having the ability to express rGAL-CS23 fusion protein.

### Example 4

Expression of Rat GALP using Optimized Sequences

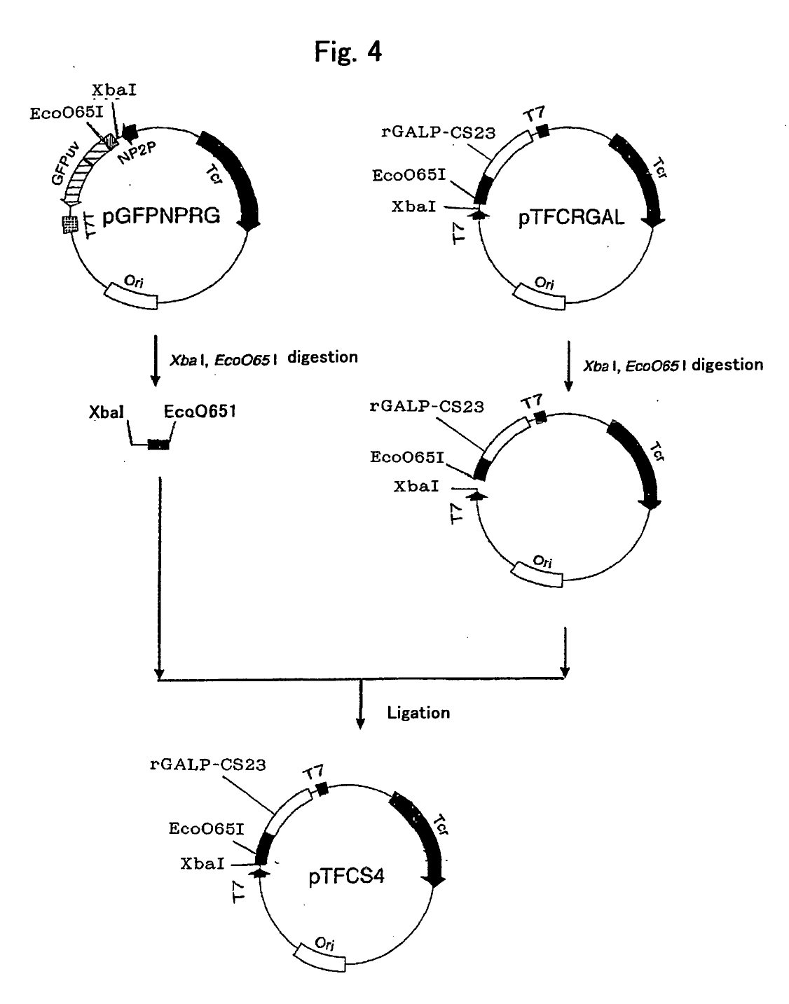

A nucleotide sequence encoding an N-terminal portion of the rat GALP structural gene in pTFCRGAL was changed from the corresponding wild type sequence to the nucleotide sequence obtained from an intense fluorescence-emitting colony in Example 2, as described below (FIG. 4).

Rat GALP-CS23 expression plasmid pTFCRGAL encoding the wild-type nucleotide sequence was digested with XbaI and EcoO65I, followed by agarose gel electrophoresis to cut out an approx. 4.5 kbp band. The DNA was recovered from the gel using QIAquick Gel Extraction Kit (Qiagen). Colony No. 1 obtained in Example 2 was inoculated into LB medium containing 10 mg/L tetracycline and cultured overnight-at 37° C. From the resultant cells, plasmid pGFPNP/rGAL1 was purified using QIAprep8 Miniprep Kit (Qiagen). This plasmid pGFPNP/rGAL1 was digested with XbaI and EcoO65I, followed by agarose gel electrophoresis to cut out an approx. 0.1 kbp band. The DNA was recovered from the gel using QIAquick Gel Extraction Kit (Qiagen). This DNA fragment was ligated to pTECRGAL between the XbaI and EcoO65I sites using Ligation kit ver. 2 (Takara Shuzo) to thereby obtain plasmid pTFCS4. This is an expression plasmid for rat GALP-CS23 fusion protein having the nucleotide sequence obtained from clone No. 1 selected in Example 2 at an N-terminal portion of rat GALP.

E. coli MM294 (DE3) was transformed with this expression plasmid pTFCS4 to thereby obtain E. coli MM294 (DE3)/pTFCS4 having the ability to express rGALP-CS4 fusion protein.

### Example 5

Comparison of Expression Levels of Rat GALP Proteins Encoded by Wild-Type and Optimized Sequences

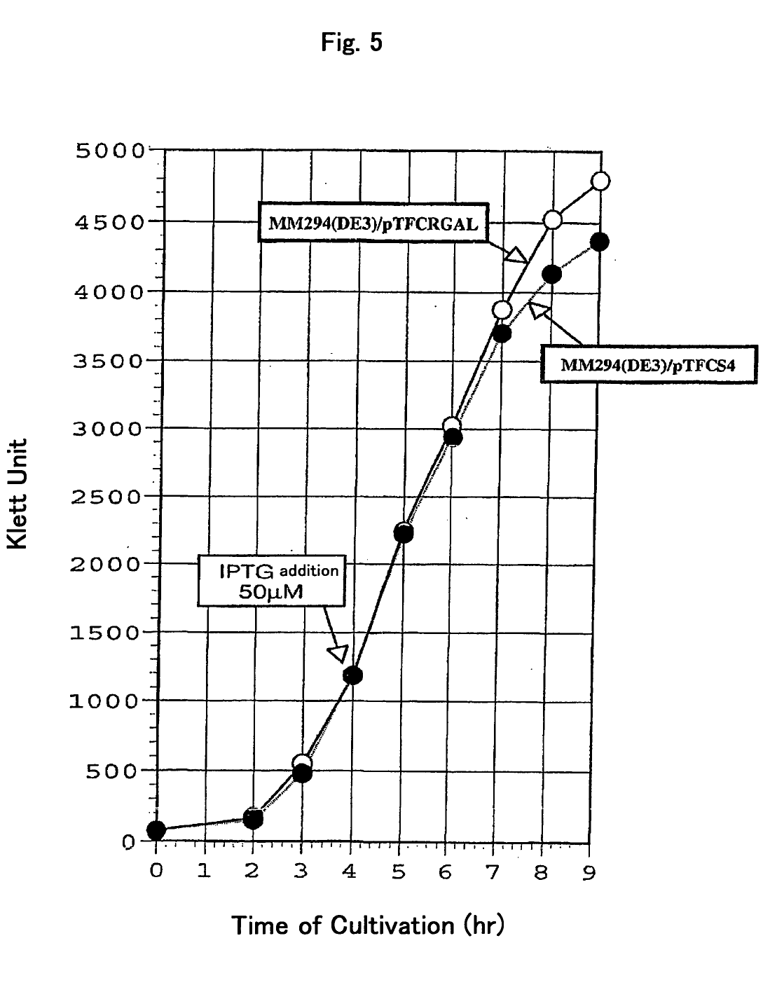

The rat GALP-CS23 fusion protein-expressing E. coli MM294 (DE3)/pTFCRGAL and E. coli MM294 (DE3)/pTFCS4 obtained in Examples 3 and 4, respectively, were cultured separately in 1 L of LB medium (1% peptone, 0.5% yeast extract, 0.5% sodium chloride) containing 10 mg/L tetracycline at 30° C. for 16 hr. The resultant culture broth (75 ml each) was transferred to a 3-L jar fermenter containing 1.5 L of a medium for primary fermentation (1.68% sodium monohydrogenphosphate, 0.3% sodium dihydrogenphosphate, 0.1% ammonium chloride, 0.05% sodium chloride, 0.024% magnesium sulfate, 0.02% New Pole LB-625, 0.0005% thianiine hydrochloride, 1.5% glucose, 1.0% casarnino acids, 1.0% yeast extract). Then, aeration agitation culture was started at 37° C. under a gas flow of 16 L/min at an agitation speed of 200 rpm. When the turbidity of the culture broth reached approx. 1200 Klett units, isopropyl-β-D-thiogalactopyranoside (IPTG) was added at 5.95 mg/l min. Twenty-four min, 130 min and 200 min after the addition of IPTG, 0.75% glucose was added. The cultivation was continued up to 9 hr after the start of the cultivation (FIG. 5).

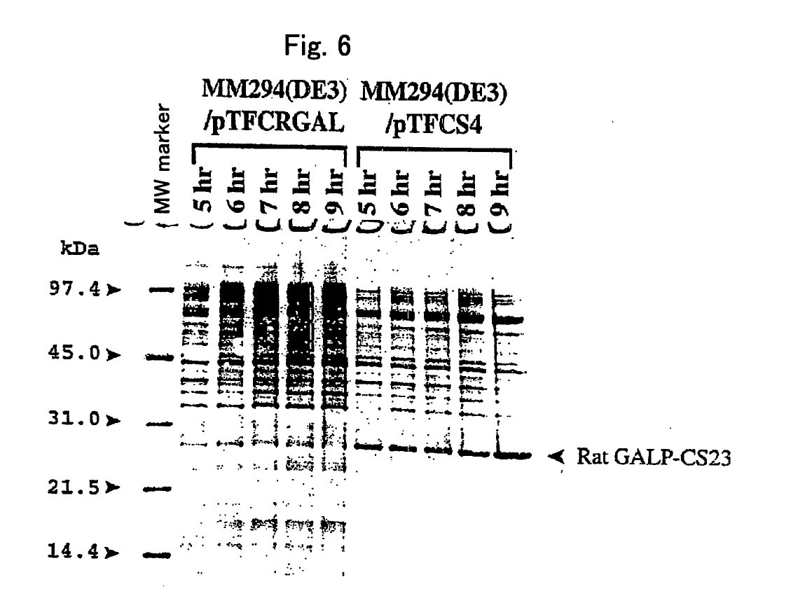

A 0.2 ml sample was taken from the culture broth at regular intervals during the cultivation and centrifuged at 12000 rpm for 10 min. The supernatant was discarded, and the resultant cells were subjected to SDS-PAGE to confirm the expression of rat GALP-CS23. Briefly, the cells obtained from 0.2 ml of the culture broth were suspended in a sample buffer (125 mM Tris-HCl, 1% sodium dodecyl sulfate, 15% glycerol, 5% 2-mercaptoethanol, 0.005% Bromophenol Blue) and treated at 95° C. for 5 min, followed by electrophoresis in Multigel 15/25 (Daiichi Pure Chemicals Co., Ltd.). The resultant gel was stained with Rapid CBB KANTO (Kanto Kagaku). As a result, a higher expression of rat GALP-CS23 was recognized in E. coli MM294 (DE3)/pTFCS4 carrying an expression plasmid in which an N-terminal portion of rat GALP is encoded by the nucleotide sequence of clone No. 1 selected in Example 2, compared to E. coli MM294 (DE3)/pTFCRCGAL carrying an expression plasmid in which the corresponding N-terminal portion of rat GALP is encoded by the wild-type sequence (FIG. 6).

### Example 6

Search for MPIF-1Δ23 Sequences Optimized for Expression

Plasmid pGFPNP was digested with NdeI (Takara Shuzo) and ScaI (Takara Shuzo), followed by agarose gel electrophoresis to cut out an approx. 4.9 kbp band. The DNA was recovered from the gel using QIAquick Gel Extraction Kit (Qiagen).

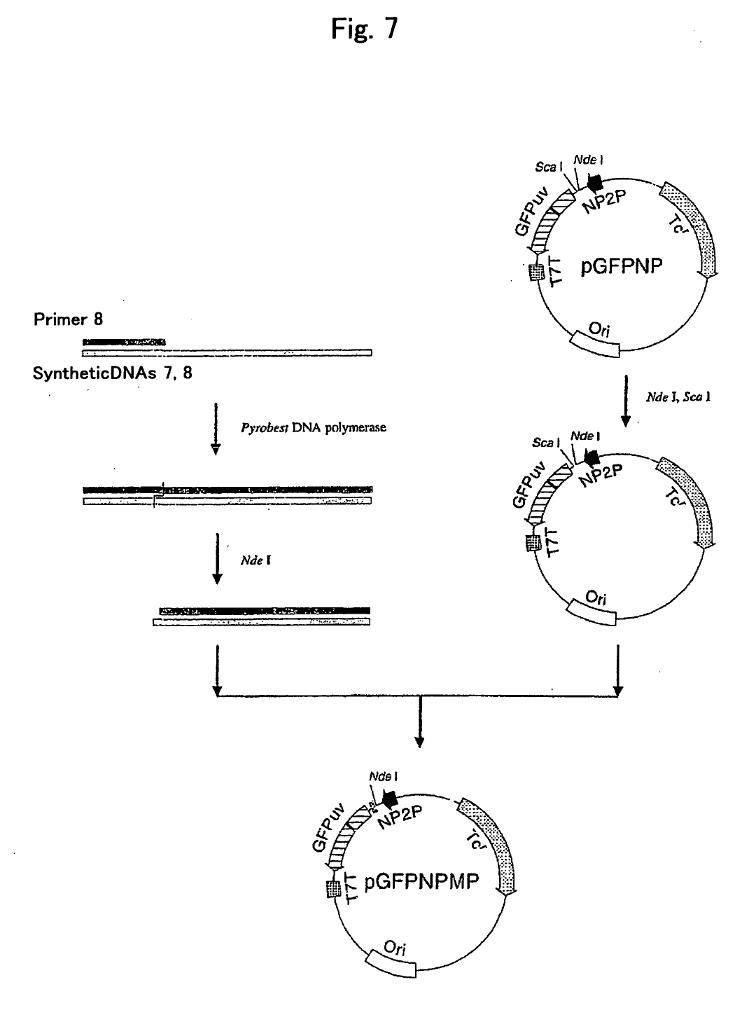

Random nucleotide sequences encoding N-terminal amino acids of MPIF-1Δ23 (myeloid progenitor inhibitory factor-1Δ23; WO 95/17092) were prepared as described below. Briefly, double-stranded DNA fragments were prepared with Pyrobest DNA polymerase (Takara Shuzo) using a 1:2 mixture of mixed bases-containing synthetic DNA 7: GGGATGCTTCGTGGGGTGTAGGAGATGCAGCAGTCAGCACTAGTNGCRTGRAAYCTRTCCATATGTTGGCGCGCCTR (SEQ ID NO: 14) and synthetic DNA 8: GGGATGCTTCGTGGGGTGTAGGAGATGCAGCAGTCAGCACTAGTNGCRTGRAANCGRTCCATATGTTGGCGCGCCTT (SEQ ID NO: 15) as templates and primer 8: AAGGCGCGCCAACATATG (SEQ ID NO: 16). The extension reaction solution was composed of 3 μl of Pyrobest DNA polymerase (5 U/μl), 10 μl of μl 10× Reaction buffer attached to the polymerase, 16 μl of dNTP mixture, 0.5 μl of synthetic DNA 7 (1 μg/μl), 1 μl of synthetic DNA 8 (1 μg/μl), 2 μl of primer 8 (100 μmol/L), and 67.5 μl of distilled water. The extension reaction was performed at 94° C. for 30 sec, at 58° C. for 30 sec, and at 70° C. for 5 min. DNA fragments were purified from the resultant reaction solution using QIAquick Gel Extraction Kit (Qiagen), followed by digestion of the double-stranded DNA fragments with NdeI (Takara Shuzo). This digestion was conducted as described below. Briefly, a reaction solution consisting of 27 μl of the purified DNA fragment solution, 3.5 μl of Buffer H (Takara Shuzo), 1.5 μl of distilled water and 3 μl of NdeI was retained at 37° C. for 2 hr to perform digestion. Then, the reaction solution was retained at 70° C. for 15 min to deactivate NdeI and concentrated/desalted with Microcon 10 (Nihon Millipore). The resultant DNA fragments were individually inserted into pGFPNP between the NdeI and ScaI sites using Ligation kit ver. 2 (Takara Shuzo) to thereby construct a set of plasmid pGFPNPMP for searching for optimized nucleotide sequences (FIG. 7).

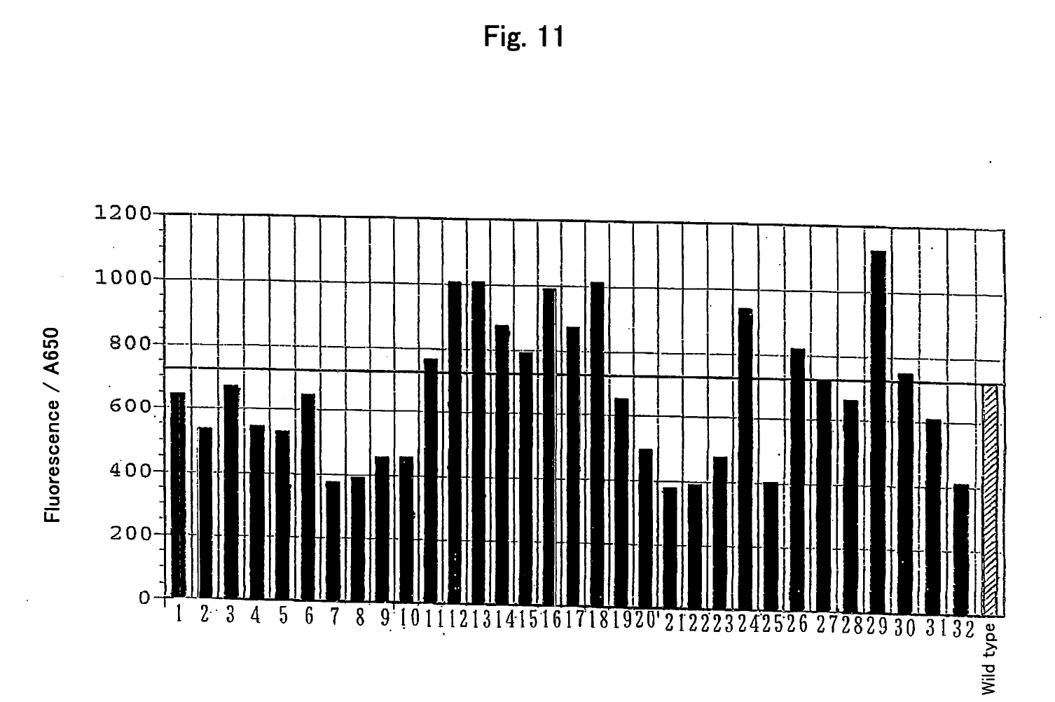

E. coli JM109 was transformed with this set of plasmid and coated on LB agar medium containing 10 mg/L tetracycline, and cultured overnight at 37° C. for colony formation. The thus formed colonies were observed under a UV ramp at 354 nm, followed by selection of 2 colonies emitting intense fluorescence, 15 colonies emitting weak fluorescence and 1 colony emitting no fluorescence. These colonies were injected separately into 0.5 ml of LB medium containing 10 mg/L tetracycline. After an overnight culture at 37° C., 50 ml of the culture broth of each colony was transferred into a 96-well microplate containing 150 ml of physiological saline. Then, absorbance at 650 nm (A₆₅₀) was measured with 96 Microplate Reader (Molecular Device), and fluorescence intensity was measured at the excitation wavelength of 355 nm and the emission wavelength of 538 nm with a multiplate fluorescence measurement apparatus (Dainippon Pharmaceutical). The value obtained by dividing fluorescence intensity by absorbance is shown as GFP expression efficiency. Furthermore, these colonies were subjected to colony PCR using primer 4 and primer 5, followed by agarose gel electrophoresis to confirm that a DNA fragment of interest was inserted. The products of the colony PCR were purified using QIAquick PCR Purification Kit (Qiagen), followed by analysis of their nucleotide sequences with primer 4 and primer 5. As a result, the nucleotide sequences shown in Table 2 were obtained. Some clones exhibited more intense fluorescence than the clone having the wild-type nucleotide sequence of MPIF-1Δ23 (Table 2, FIG. 11).

TABLE 2

| | | | | | | | | Fluorescence |
|---|---|---|---|---|---|---|---|---|
| Wild | Residue | 1 | 2 | 3 | 4 | 5 | GC content | intensity/ |
| type | Amino acid | Asp | Arg | Phe | His | Ala | (%) | Turbidity |
| sequence | Nucleotide | GAC | AGA | TTC | CAC | GCT | 46.7 | 723 |
| Nucleotide sequences | 1 | GAC | AGA | TTC | CAC | GCA | 53.3 | 644 |
| obtained from E. coli clones | 2 | GAC | AGA | TTC | CAC | GCC | 60.0 | 533 |
| | 3 | GAC | AGA | TTC | CAT | GCA | 46.7 | 669 |
| | 4 | GAC | AGA | TTT | CAC | GCA | 46.7 | 543 |
| | 5 | GAC | AGA | TTT | CAC | GCC | 53.3 | 526 |
| | 6 | GAC | AGA | TTT | CAC | GCG | 53.3 | 646 |
| | 7 | GAC | AGG | TTC | CAC | GCA | 60.0 | 375 |
| | 8 | GAC | AGG | TTC | CAC | GCG | 66.7 | 391 |
| | 9 | GAC | AGG | TTT | CAC | GCT | 60.0 | 455 |
| | 10 | GAC | AGG | TTT | CAT | GCC | 53.3 | 456 |
| | 11 | GAC | CGA | TTC | CAC | GCG | 66.7 | 765 |
| | 12 | GAC | CGA | TTC | CAC | GCT | 60.0 | 1005 |
| | 13 | GAC | CGA | TTC | CAC | GCC | 60.0 | 1006 |
| | 14 | GAC | CGA | TTC | CAT | GCG | 60.0 | 874 |
| | 15 | GAC | CGA | TTC | CAT | GCT | 53.3 | 791 |
| | 16 | GAC | CGA | TTT | CAT | GCC | 53.3 | 988 |
| | 17 | GAC | CGC | TTC | CAC | GCC | 73.3 | 872 |
| | 18 | GAC | CGC | TTC | CAC | GCG | 73.3 | 1013 |
| | 19 | GAC | CGC | TTC | CAC | GCT | 66.7 | 653 |
| | 20 | GAC | CGC | TTT | CAC | GCA | 60.0 | 497 |
| | 21 | GAC | CGG | TTC | CAC | GCA | 66.7 | 378 |
| | 22 | GAC | CGG | TTC | CAT | GCC | 66.7 | 391 |
| | 23 | GAC | CGG | TTT | CAC | GCA | 60.0 | 477 |
| | 24 | GAT | AGA | TTC | CAC | GCT | 46.7 | 940 |
| | 25 | GAT | AGA | TTT | CAC | GCC | 46.7 | 403 |
| | 26 | GAT | AGA | TTT | CAC | GCG | 46.7 | 821 |
| | 27 | GAT | AGA | TTT | CAC | GCT | 40.0 | 718 |
| | 28 | GAT | AGG | TTC | CAC | GCT | 53.3 | 662 |
| | 29 | GAT | CGA | TTT | CAC | GCA | 53.3 | 1126 |
| | 30 | GAT | CGC | TTC | CAC | GCA | 66.7 | 751 |
| | 31 | GAT | CGC | TTT | CAC | GCT | 53.3 | 610 |
| | 32 | GAT | CGG | TTC | CAC | GCA | 60.0 | 412 |

### Example 7

Construction of Plasmid pNPHGHlacts

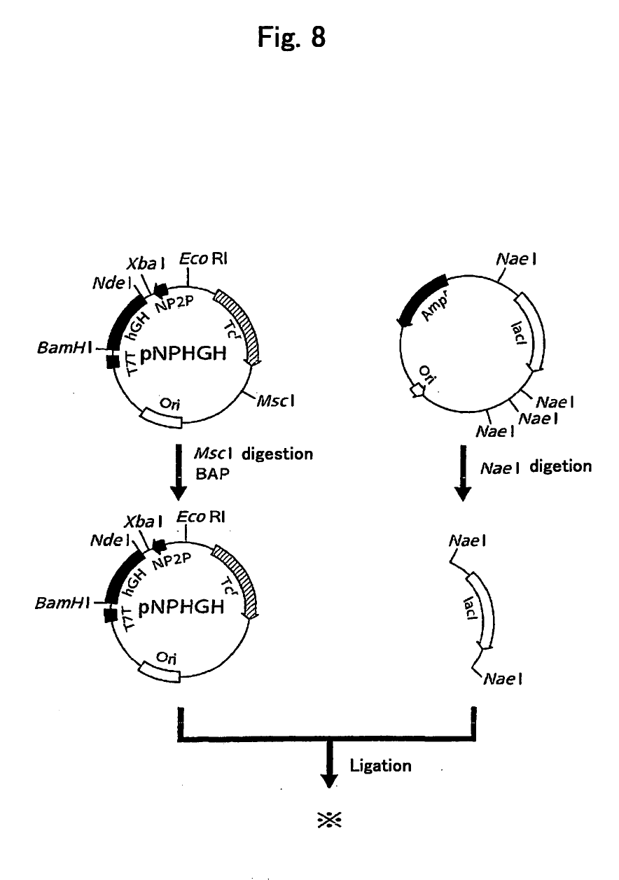

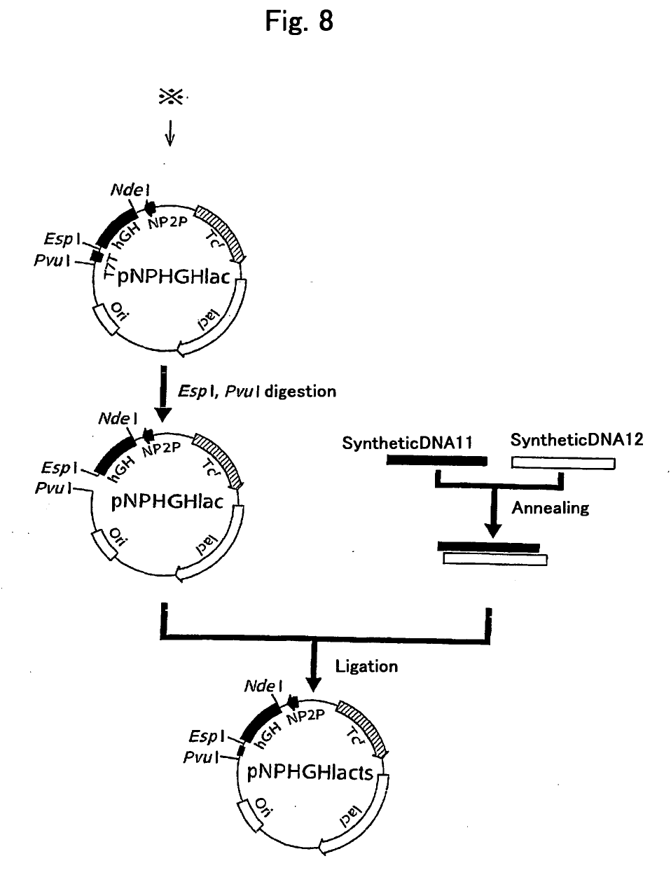

Plasmid pNPHGH constructed in Example 1 was digested with MscI, followed by dephosphorylation of the resultant ends with Bacterial Alkaline Phosphatase (Nippon Gene). On the other hand, plasmid pET11a (Takara Shuzo) was digested with NaeI, followed by agarose gel electrophoresis to cut out an approx. 1.6 kbp band. The DNA was recovered from the gel using QIAquick Gel Extraction Kit (Qiagen). This DNA fragment containing lacI was inserted into the MscI-digested site of pNPHGH using Ligation kit ver. 2 (Takara Shuzo) to thereby obtain plasmid pNPHGHlac. Further, the following operations were conducted in order to replace its T7 terminator with a synthetic terminator. Briefly, plasmid pNPHGHlac was digested with EspI and PvuI, followed by agarose gel electrophoresis to cut out an approx. 6.1 kbp band. The DNA was recovered from the gel using QIAquick Gel Extraction Kit (Qiagen). One hundred pmol each of synthetic DNA 9: TGAGCATGCATACTAGTCTCGAGTAATCCCACAGCCGCCGCCAGTTCCGCTGGCGGCGGCATTTTCGAT (SEQ ID NO: 17) and synthetic DNA 10: CGAAAATGCCGCCGCCAGCGGAACTGGCGGCGGCTGTGGGATTACTCGAGACTAGTATGCATGC (SEQ ID NO: 18) were dissolved in TE buffer, retained at 90° C. for 10 min, and then gradually cooled to room temperature to perform annealing. The resultant DNA was ligated to pNPHGHlac between the EspI and PvuI sites using Ligation kit ver. 2 (Takara Shuzo) to thereby obtain plasmid pNPHGHlacts (FIG. 8).

### Example 8

Construction of Clones that Express MPIF-1Δ23 Encoded by Wild Type Nucleotide Sequence

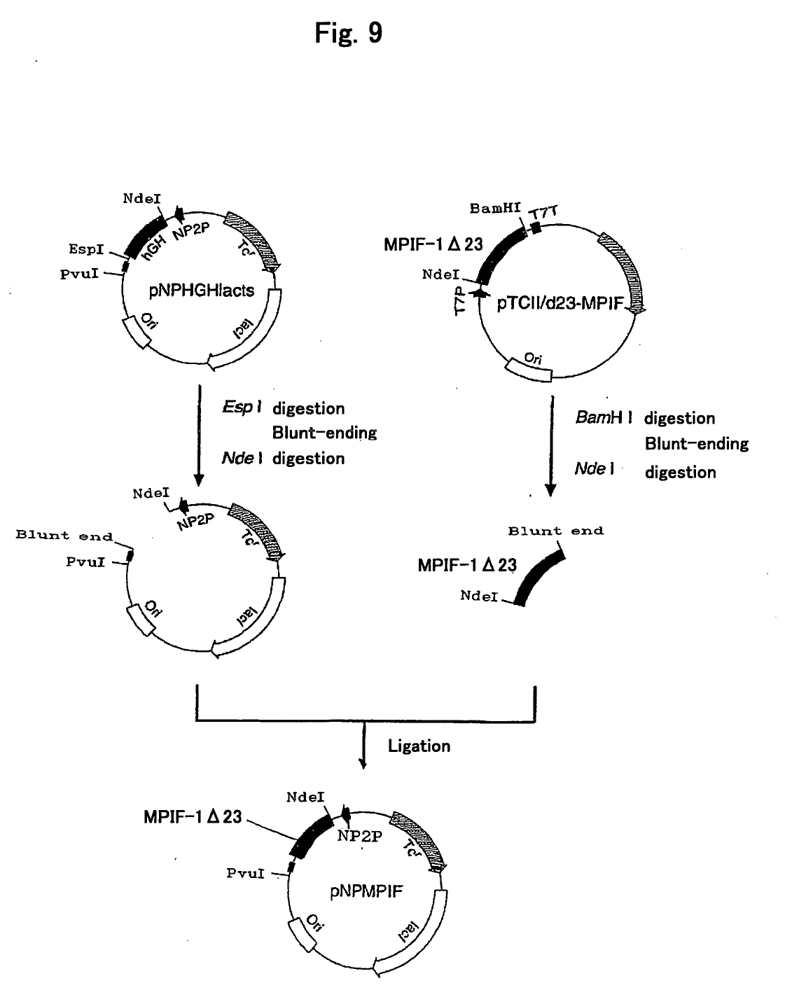

Plasmid pNPHGHlacts was digested with EspI, followed by blunt-ending with DNA Blunting Kit (Takara Shuzo) and further digestion with NdeI. The digest was subjected to agarose gel electrophoresis to cut out an approx. 3.9 kbp band, and the DNA was recovered from the gel using QIAquick Gel Extraction Kit (Qiagen). Plasmid pTCII/d23-MPIF1 described in Reference Example 1 in WO 00/40610 was digested with BamHI, followed by blunt-ending with DNA Blunting Kit (Takara Sbuzo) and further digestion with NdeI. The digest was subjected to agarose gel electrophoresis to cut out an approx. 240 bp band, and the DNA was recovered from the gel using QIAquick Gel Extraction Kit (Qiagen). This DNA was ligated to pNPHGHlacts between the NdeI site and EspI (blunted) site using Ligation kit ver. 2 (Takara Shuzo) to thereby obtain plasmid pNPMPIF for expressing MPIF-1Δ23 encoded by the wild-type sequence (FIG. 9). E. coli JM109 competent cells (Toyobo) were transformed with this plasmid to thereby obtain E. coli JM 109/pNPMPIF having the ability to express MPIF-1Δ23.

### Example 9

Construction of Clones that Express MPIF-1Δ23 Encoded by Optimized Sequences

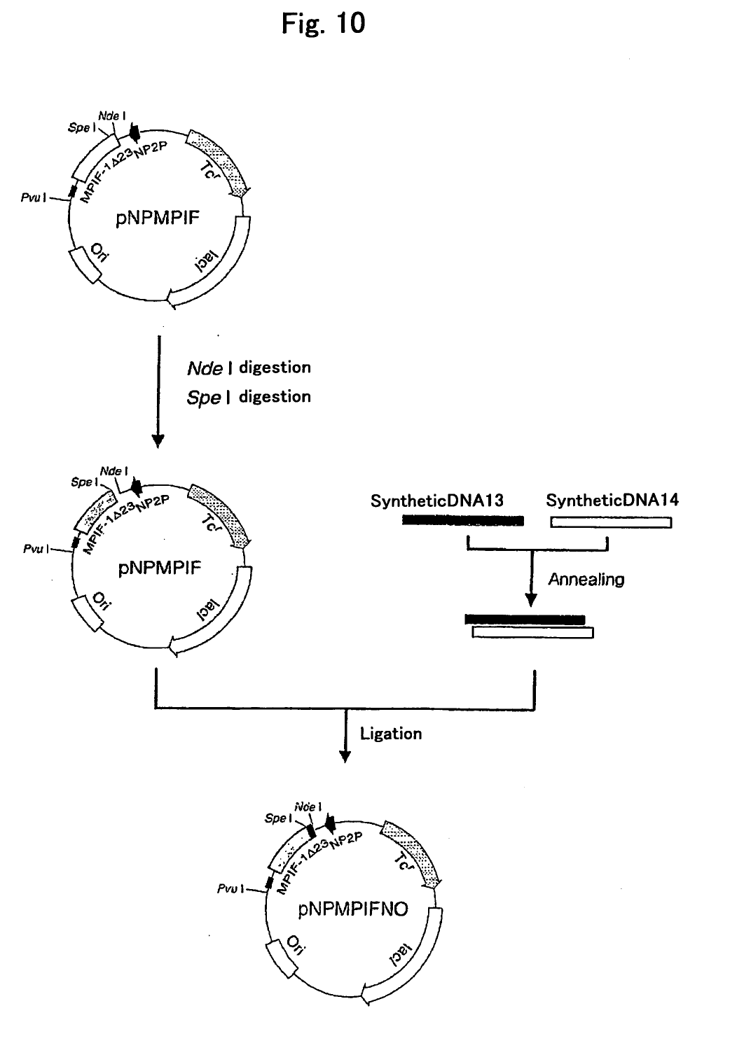

Plasmid pNPMPIF obtained in Example 8 was digested with NdeI (Takara Shuzo) and SpeI (Takara Shuzo), followed by agarose gel electrophoresis to cut out an approx. 4.8 kbp band. The DNA was recovered from the gel using QIAquick Gel Extraction Kit (Qiagen). One hundred pmol each of synthetic DNA 11: TATGGATCGATITCACGCAA (SEQ ID NO: 19) comprising the nucleotide sequence of clone No. 29 obtained in Example 7 and synthetic DNA 12: CTAGTTGCGTGAAATCGATCCA (SEQ ID NO: 20) comprising a nucleotide sequence complementary to the nucleotide sequence of clone No. 29 were dissolved in TE buffer, retained at 90° C. for 10 min, and then gradually cooled to room temperature to perform annealing. The resultant double-stranded DNA fragment was ligated to pTCII/d23-MPIF between the NdeI and SpeI sites using Ligation kit ver. 2 (Takara Shuzo) to thereby obtain plasmid pTCII/MPIFNO. pTCII/MPIFNO was digested with BamHI, followed by blunt-ending with DNA Blunting Kit (Takara Shuzo) and further digestion with NdeI. The digest was subjected to agarose gel electrophoresis to cut out an approx. 240 bp band, and the DNA was recovered from the gel using QIAquick Gel Extraction Kit (Qiagen). This DNA was ligated to pNPHGHkacts between the NdeI site and EspI (blunted) site using Ligation kit ver. 2 (Takara Shuzo) to thereby obtain plasmid pTCII/MPIFNO for expressing a MPIF-1Δ23 protein having an N-terminal portion encoded by a modified nucleotide sequence (FIG. 10). This plasmid comprises a DNA (SEQ ID NO: 120) that comprises the nucleotide sequence of clone No. 29 in Example 6 and has a nucleotide sequence encoding MPIF-1Δ23.

E. coli JM109 competent cells (Toyobo) were transformed with this plasmid to thereby obtain E. coli JM109/pNPMPIFNO having the ability to express MPIF-1Δ23.

### Example 10

Comparison of Expression Levels of MPIF-1Δ23 Proteins Encoded by Wild-Type and Optimized Sequences

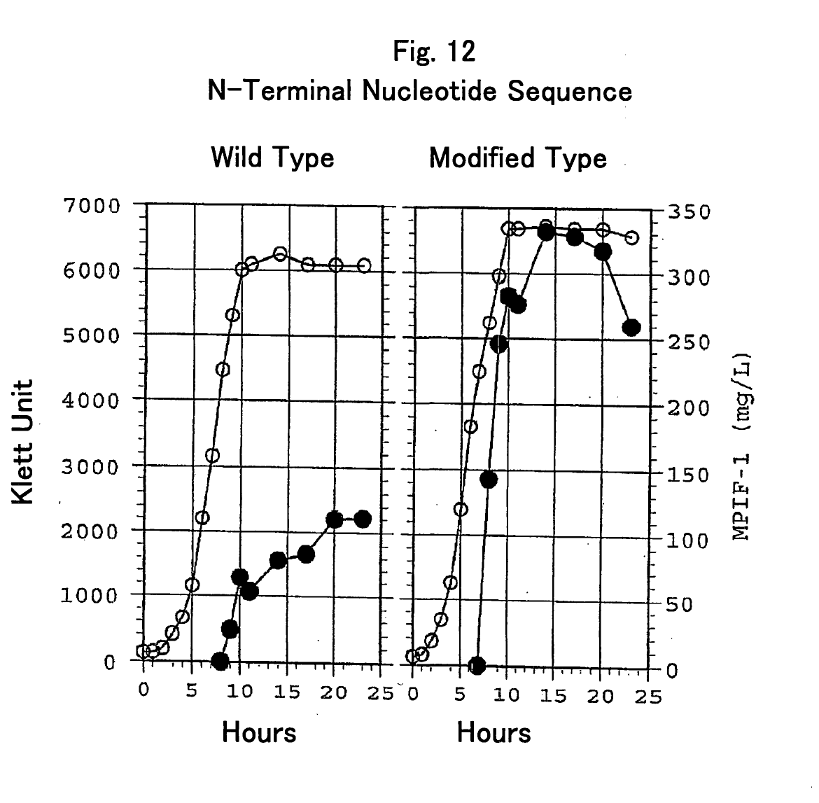

The MPIF-1Δ23-expressing E. coli JM109/pNPMPIF obtained in Example 6 and E. coli JM109/pNPMPIFNO described above were cultured separately in 1 L of LB medium (1% peptone, 0.5% yeast extract, 0.5% sodium chloride) containing 10 mg/L tetracycline at 30° C. for 16 hr. The resultant culture broth (75 ml each) was transferred to a 3-L jar fermenter containing 1.5 L of a medium for primary fermentation (1.68% sodium monohydrogenphosphate, 0.3% sodium dihydrogenphosphate, 0.1% anmmonium chloride, 0.05% sodium chloride, 0.024% magnesium sulfate, 0.02% New Pole LB-625, 0.0005% thianine hydrochloride, 1.5% glucose, 1.0% casamino acids, 1.0% yeast extract). Then, aeration agitation culture was started at 37° C. under a gas flow of 16 Lmrnin at an agitation speed of 200 rpm. When the turbidity of the culture broth reached approx. 4500 Klett units, isopropyl-β-D-thiogalactopyranoside (IPTG) was added at 5.95 mg/l min. Twenty-four min, 130 min and 200 min after the addition of IPTG, 0.75% glucose was added. The cultivation was continued up to 23 hr after the start of the cultivation. A 0.2 ml sample was taken from the culture broth at regular intervals during the cultivation and centrifuged at 12000 rpm for 10 min. The supernatant was discarded, and MPIF-1Δ23 was extracted from the resultant cells, followed by determination of MPIF-1Δ23 expression levels by ELISA. Briefly, cells obtained from 0.2 ml of the culture broth were suspended in 400 μl of 100 mM Tris-HCl (pH 7). To 50 μl of this suspension, 400 μl of an extraction buffer (100 mM Tris-HCl (pH 7)+2.5% SDS) was added. The resultant mixture was subjected to sonication for 5 min using a ultrasonic cell disruption apparatus BIORUPTOR (Olympus) to thereby extract MPIF-1Δ23. ELISA was performed as described below using an anti-MPIF-1 antibody (DAKO Japan) as a primary antibody and the same antibody biotinylated with Sulfo-NHS-Biotin (Funakoshi) as a secondary antibody. Briefly, 100 μl of a coating buffer (0.293% sodium hydrogencarbonate, 0.159% sodium carbonate sodium azide) containing 4 μg/ml of the anti-MPIF-1 antibody was dispensed into each well of a 96-well microplate (Nunc) and left overnight at 4° C. After washing each well with a washing buffer (PBS+0.1% Tween 20), 50 μl of MPIF-1Δ23 extract diluted with a dilution buffer (PBS, 10% Block Ace, 0.1% Tween 20 , 0.02% Merthiolate) was added to each well and left for 2 hr at room temperature. After washing each well with the washing buffer, 100 μl of biotinylated anti-MPIF-1 antibody diluted with the dilution buffer (2 μg/ml) was added thereto and left for 1 hr at room temperature. After washing each well with the washing buffer, 100 μl of a 500-fold dilution of alkali phosphatase streptavidin (Vector) in the washing buffer was added thereto and left for 1 hr at room temperature. After washing each well with the washing buffer, 100 μl of a 500-fold dilution of alkali phosphatase streptavidin (Vector) in the washing buffer was added thereto and left for 1 hr at room temperature. After washing each well with the washing buffer, a substrate reaction was carried out using Bluephos Microwell Phosphatase Substrate System (KPL) at 37° C. for 20 min. Then, the reaction was terminated by adding 100 μl of 2.5% EDTA4-Na solution. Absorbance at 620 nm was measured with a microplate reader (Labsystems). As a standard, an MPIF-1Δ23 protein prepared by the method described in WO 00/40610 was used. The time course of the cultivation and the results of measurement of expression levels are shown in FIG. 12. In the expression of MPIF-1Δ23 proteins, a higher expression of the protein having the nucleotide sequence selected in Example 6 by the process of the invention at its N-terminal was recognized, compared to the protein comprising the corresponding wild-type nucleotide sequence.

### Example 11

Construction of Plasmid pKMNP for Searching for High Expression Sequences

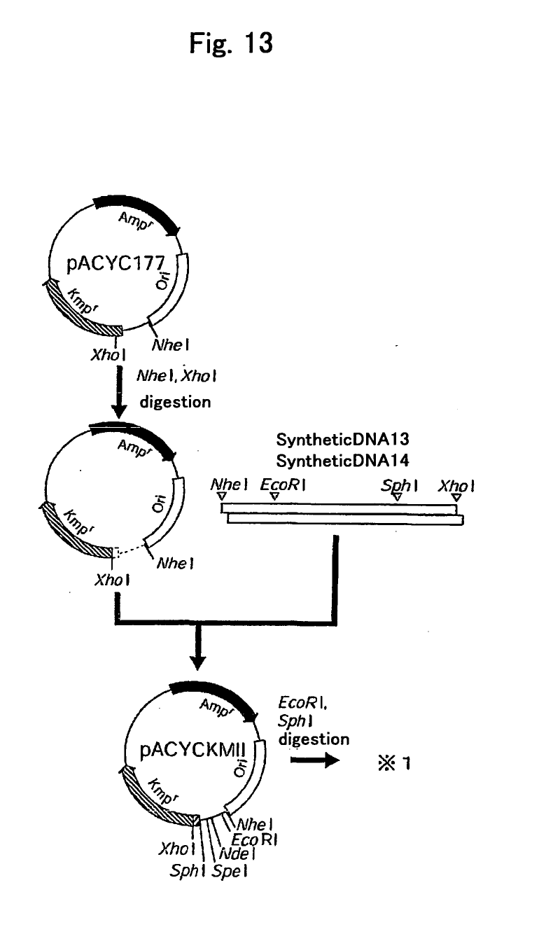

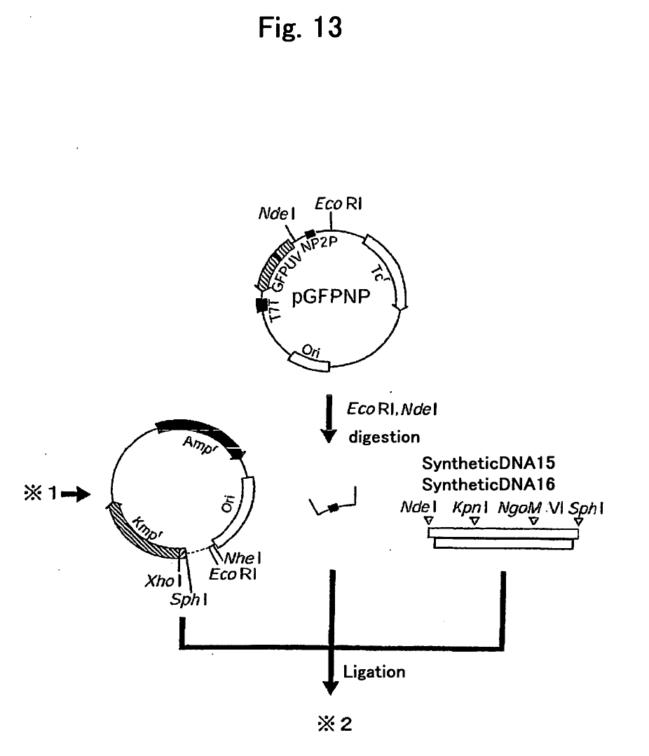

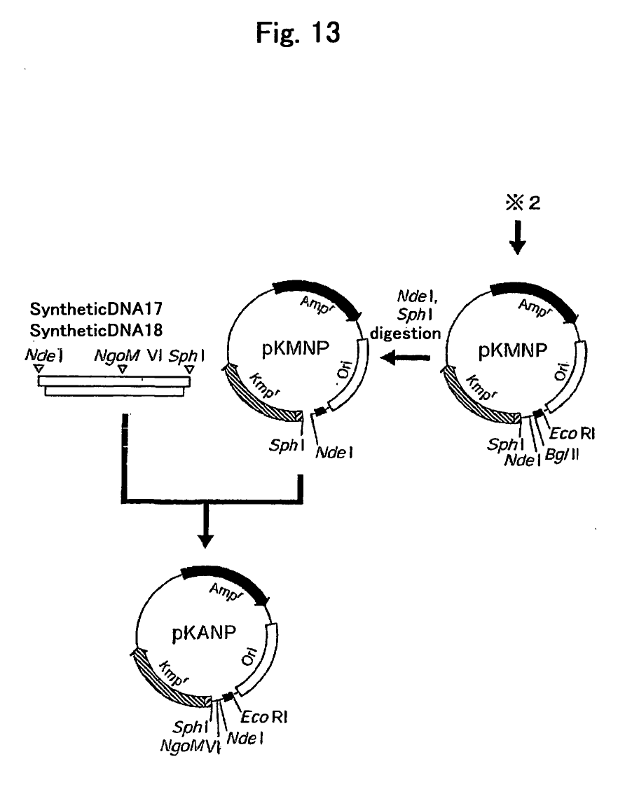

Plasmid pKMNP for searching for high expression sequences using kanamycin resistance resulted from the expression of kanamycin resistance gene was constructed as described below (FIG. 13).

Plasmid pACYC177 (Wako Purechemical Industries) was digested with restriction enzymes NheI and XhoI and subjected to agarose gel electrophoresis to cut out an approx. 3.6 kbp band. The DNA recovered from the gel using QIAquick Gel Extraction Kit (Qiagen). One hundred pmol each of synthetic DNA 13: CTAGCGAATTCGAGCATATGAGCACTAGTGCATGCGAGCCATATTCAACGGGAAACGTCTTGC (SEQ ID NO: 75) and synthetic DNA 14: TCGAGCAAGACGMTFCCCGTTGAATATGGCTCGCATGCACTAGTGCTCATATGCTCGAATTCG (SEQ ID NO: 76) were dissolved in TE buffer, retained at 90° C. for 10 min, and then gradually cooled to room temperature to perform annealing. The thus annealed DNA fragment was inserted into pACYC177 between the NheI and XhoI sites using Ligation kit ver. 2 (Takara Shuzo) to thereby obtain plasmid pACYCMII in which EcoRI and SphI restriction sites have been introduced upstream of its kanamycin resistance gene.

Plasmid pACYCMII was digested with EcoRI and SphI and subjected to agarose gel electrophoresis to cut out an approx. 3.6 kbp band. The DNA was recovered from the gel using QIAquick Gel Extraction Kit (Qiagen). Plasmid pGFPNP obtained in Example 1 was digested with EcoRI and NdeI and subjected to agarose gel electrophoresis to cut out an approx; 0.15 kbp band. The DNA was recovered from the gel using QIAquick Gel Extraction Kit (Qiagen). One hundred pmol each of synthetic DNA 15: TATGAGGGTACCGCCGGCTGCATG (SEQ ID NO: 77) and synthetic DNA 16: CAGCCGGCGGTACCCTCA (SEQ ID NO: 78) were dissolved in TE buffer, retained at 90° C. for 10 min, and then gradually cooled to room temperature to perform annealing. The thus annealed DNA fragment and the EcoRI-NdeI fragment from pGFPNP were inserted into pACYCMII between the EcoRI and SphI sites using Ligation kit ver. 2 (Takara Shuzo) to thereby obtain plasmid pKMNP.

Plasmid pKMNP was digested with NdeI and SphI, and subjected to agarose gel electrophoresis to cut out an approx. 3.8 kbp band. The DNA was recovered from the gel using QIAquick Gel Extraction Kit (Qiagen). One microgram each of synthetic DNA 17: TATGAGGGTACCGCCGGCACTGCATG (SEQ ID NO: 79) and synthetic DNA 18: CACTGCCGGCGGTACCCTCA (SEQ ID NO: 80) were reacted in 100 μl of a phosphorylation reaction solution [50 mM Tris-HCl (pH 7.6), 10 mM MgCl₂, 1 mM spermidine, 10 mM dithiothreitol, 0.1 mg/ml bovine serum albumin, 1 mM ATP, 10 units of T4 polynucleotide kinase (Nippon Gene)] for 1 hr at 37° C. to phosphorylate the 5′ end. Then, the reaction solution was retained at 90° C. for 10 min and gradually cooled to room temperature to perform annealing. The resultant DNA fragment was inserted into pKMNP between the NdeI and SphI sites using Ligation kit ver. 2 (Takara Shuzo) to thereby obtain plasmid pKANP. By inserting random nucleotide sequences encoding an N-terminal amino acid sequence of the protein of interest between the NdeI and NgoMIV sites of plasmid pKANP located upstream of its kanamycin resistance structural gene, it is possible to select sequences of high expression efficiency using kanamycin resistance as an indicator.

### Example 12

Construction of an Expression Plasmid for ZAQ-1 Ligand Encoded by Wild Type Nucleotide Sequence

A ZAQ-1 ligand expression plasmid whose structural gene is a wild type nucleotide sequence was constructed as described below.

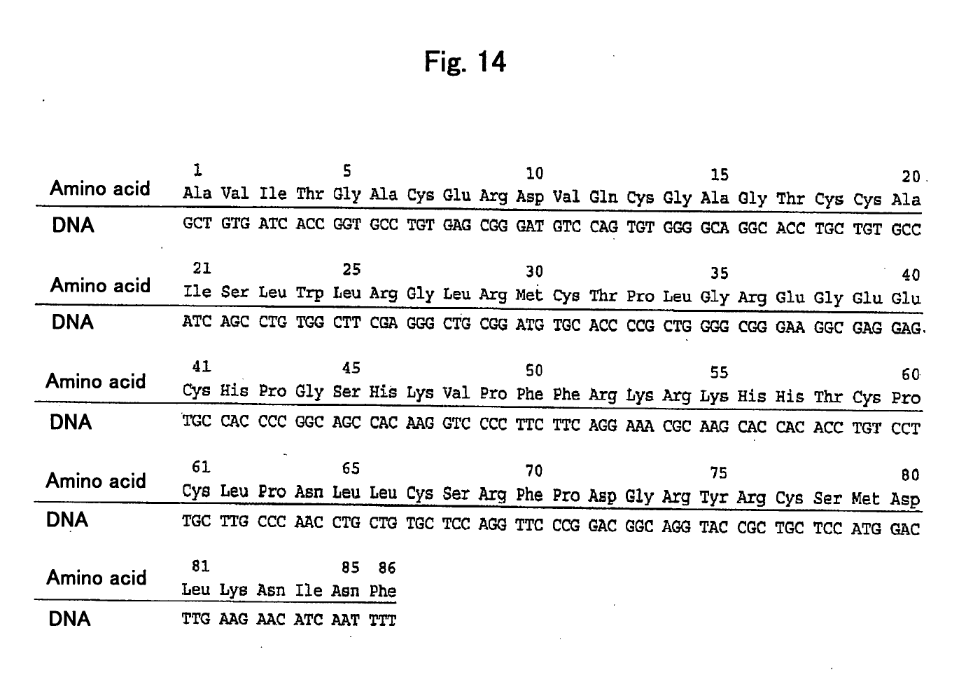

A ZAQ-1 ligand structural gene for which the amino acid sequence and the wild type nucleotide sequence are shown in FIG. 14 was amplified from plasmid pHMITG by PCR using synthetic DNA 19: CTCATATGGCTGTGATCACCGGTGCCTGTG (SEQ ID NO: 81) located immediately upstream of the structural gene and having an NdeI restriction site and Met, and synthetic DNA 20: GCGGATCCCTAAAAATTGATGTTCTTCAAG (SEQ ID NO: 82) located immediately downstream of the structural gene and having a termination codon and a BamHI restriction site. This gene was ligated to pCRII-TOPO vector using TOPO TA Cloning Kit (Invitrogen) to thereby prepare pCRII/GAL. This plasmid was transformed into TOP10 One Shot Competent Cells and plated on X-gal (5-bromo4-chloro-3-indolyl-β-D-galactose)-coated LB agar medium (1% peptone, 0.5% yeast extract, 0.5% sodium chloride, 2% agar) containing 50 μg/ml ampicillin, followed by cultivation at 37° C. for one day. Then, transformants were selected using tetracycline resistance and β galactosidase activity as indicators. The transformant was cultured overnight in LB medium (1% peptone, 0.5% yeast extract, 0.5% sodium chloride) containing 50 μg/ml ampicillin, followed by recovery of plasmid pCRII-TOPOh1ZAQ using QIAprep8 Miniprep Kit (Qiagen). This plasmid pCRII-TOPOh1ZAQ was digested with restriction enzymes NdeI and BamHI, and subjected to agarose gel electrophoresis to cut out an approx. 0.3 kbp band. The DNA was recovered from the gel using QIAquick Gel Extraction Kit (Qiagen).

Plasmid pTCII described in WO 00/40610 was digested with restriction enzymes NdeI and BamHI, and subjected to agarose gel electrophoresis to cut out an approx. 4.6 kbp band. The DNA was recovered from the gel using QIAquick Gel Extraction Kit (Qiagen). The DNA fragment prepared above comprising the ZAQ-1 ligand structural gene was ligated to plasmid pTCII between the NdeI and BamHI sites using Ligation kit ver. 2 (Takara Shuzo) to thereby prepare an expression plasmid pTCIIZAQN for expressing ZAQ-1 ligand having the wild type nucleotide sequence.

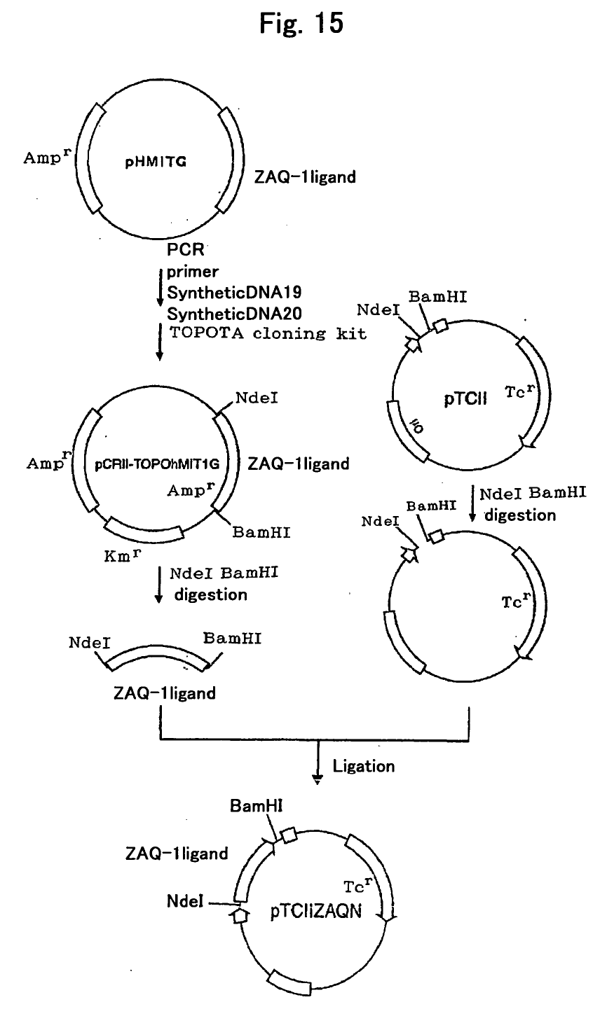

Plasmid pTCIIZAQN was transformed into E. coli MM294 (DE3) cells, which were plated on LB agar medium containing 10 μg/ml tetracycline and cultured at 37° C. for one day. As a result, clone MM294 (DE3)/pTCIIZAQN was obtained that expresses a ZAQ-1 ligand having a nucleotide sequence with highly frequently used codons (FIG. 15).

### Example 13

Construction of an Expression Plasmid for ZAQ-1 Ligand Encoded by Nucleotide Sequence with Highly Frequently Used Codons

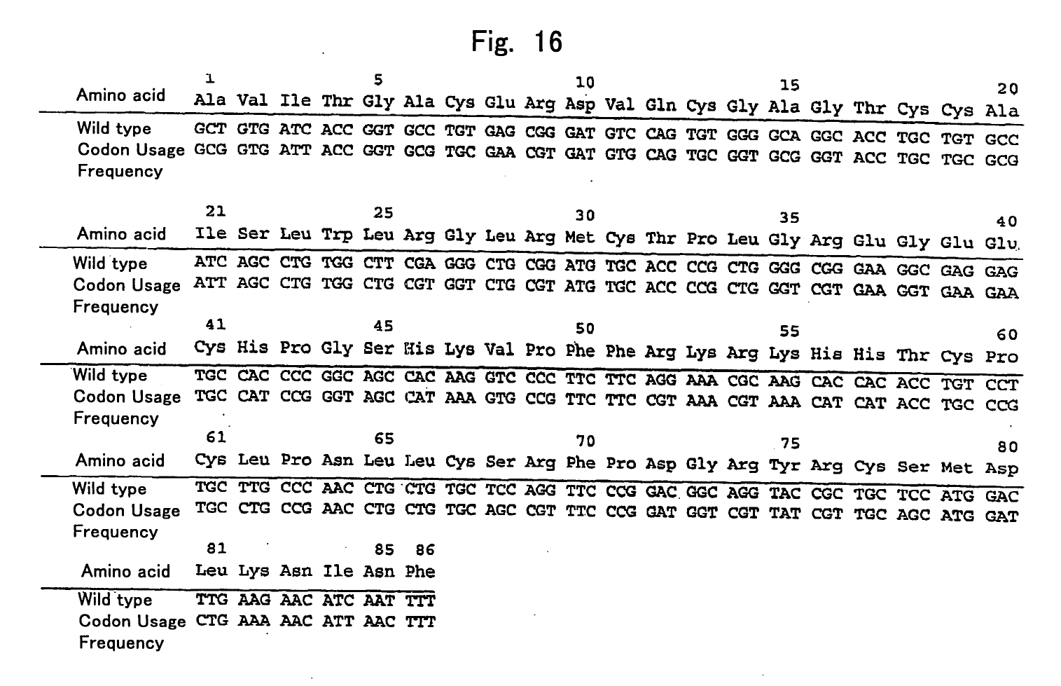

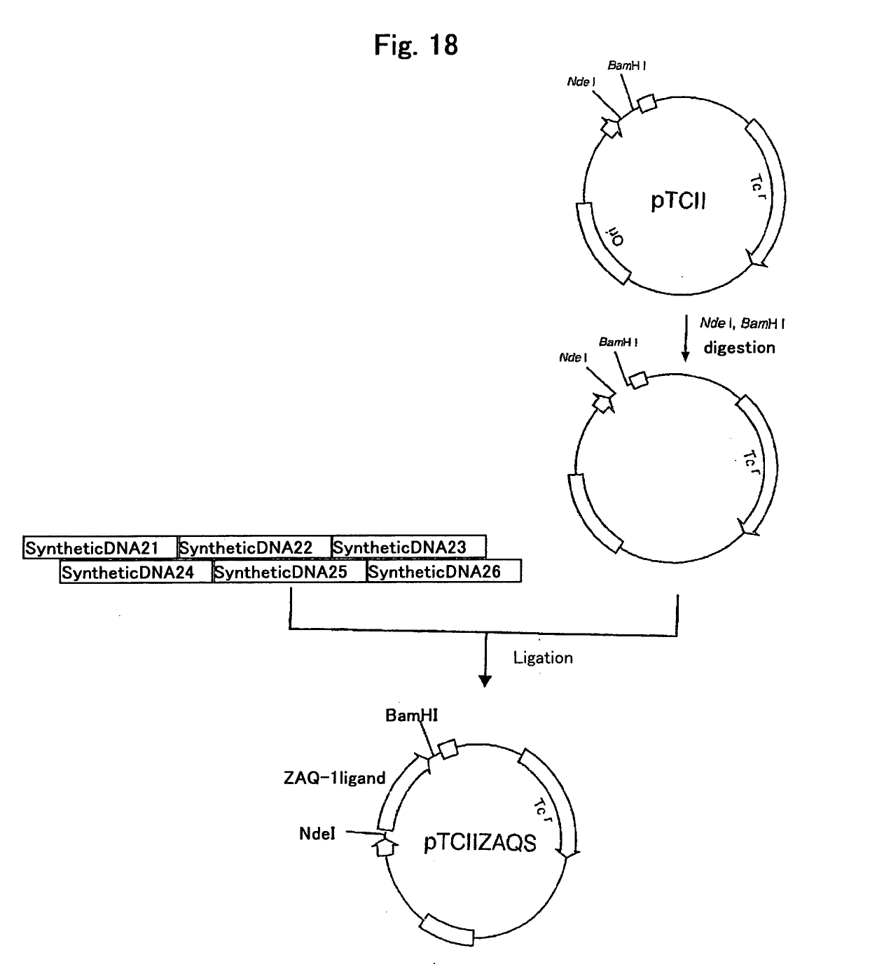

An expression plasmid was constructed that has a structural gene comprising those codons used highly frequently in E. coli. With reference to the frequency of usage of each codon in E. coli disclosed in Codon Usage Database (http://www.kazusa.or.jp/codon/), the nucleotide sequence shown in FIG. 16 was prepared and used as a ZAQ-1 ligand structural gene. In FIG. 16, the upper row shows the amino acid sequence of ZAQ-1 ligand; the middle row shows the wild-type nucleotide sequence encoding ZAQ-1 ligand and the bottom row shows the nucleotide sequence comprising highly frequently used codons. The ZAQ-1 ligand structural gene was prepared as described below (FIG. 18).

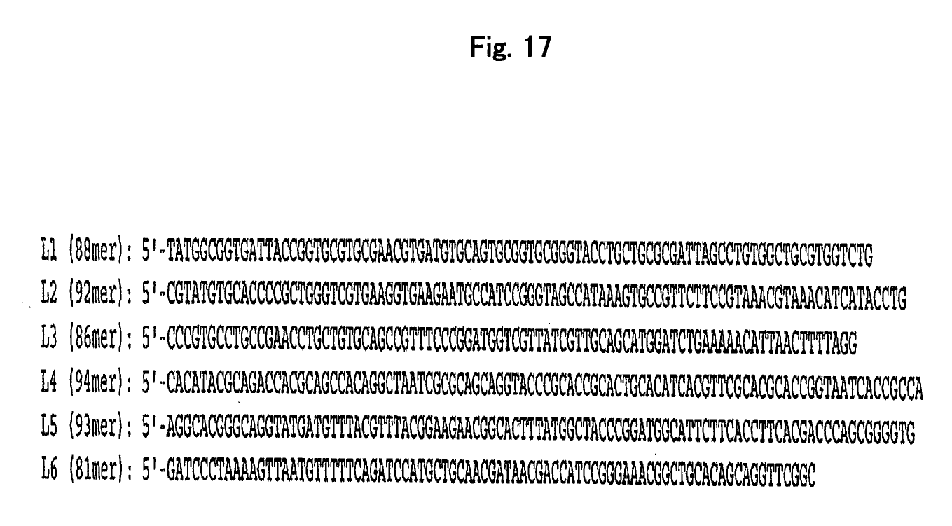

The six DNA fragments shown in FIG. 17 were used: synthetic DNA 21: TATGGCGGTGATTACCGGTGCGTGCGAACGTGATGTGCAGTGCGGTGCGGGTACCTGCTGCGCGATTAGCCTGTGGCTGCGTGGTCTG (SEQ ID NO: 83), synthetic DNA 22: CGTATGTGCACCCCGCTGGGTCGTGAAGGTGAAGAATGCCATCCGGGTAGCCATAAAGTGCCGTTCTTCCGTAAACGTAAACATCATACCTG (SEQ ID NO: 84), synthetic DNA 23: CCCGTGCCTGCCGAACCTGCTGTGCAGCCGTTTCCCGGATGGTCGTTATCGTTGCAGCATGGATCTGAAAAACATTAACTFTTAGG (SEQ ID NO: 85), synthetic DNA 24: CACATACGCAGACCACGCAGCCACAGGCTAATCGCGCAGCAGGTACCCGCACCGCACTGCACATCACGTTCGCACGCACCGGTAATCACCGCCA (SEQ ID NO: 86), synthetic DNA 25: AGGCACGGGCAGGTATGATGTTFACGTTTACGGAAGAACGGCACTTTATGGCTACCCGGATGGCATTCTTCACCTTCACGACCCAGCGGGGTG (SEQ ID NO: 87), and synthetic DNA 26: GATCCCTAAAAGTTAATGTTTTTCAGATCCATGCTGCAACGATAACGACCATCCGGGAAACGGCTGCACAGCAGGTTCGGC (SEQ ID NO: 88).

a) Phosphorylation of DNA Oligomers

One microgram each of the four synthetic DNAs (excluding synthetic DNAs 21 and 26 that are to form 5′ ends) were reacted in 100 μl of a phosphorylation reaction solution [50 mM Tris-HCl (pH 7.6), 10 mM MgCl₂, 1 mM spermidine, 10 mM dithiothreitol, 0.1 mg/ml bovine serum albumin, 1 mM ATP, 10 units of T4 polynucleotide kinase (Nippon Gene)] for 1 hr at 37° C. to phosphorylate the 5′ ends. After phenol treatment, the aqueous layer was recovered. Two volumes of ethanol was added thereto and cooled to −70° C. Then, the DNA was precipitated by centrifugation.

b) Ligation of DNA Fragments

The phosphorylated DNA fragments obtained in a) above and 1 μg each of synthetic DNAs 21 and 26 were mixed to give a mixed solution of 120 μl. This mixed solution was retained at 80° C. for 10 min and then gradually cooled to room temperature to perform annealing. A ligation reaction was performed using TaKaRa DNA Ligation Kit ver. 2 (Takara Shuzo). Briefly, 30 μl of solution II was added to 30 μl of the annealing solution and mixed well; then, 60 μl of solution I was added thereto and reacted at 37° C. for 1 hr to perform ligation. After phenol treatment, the aqueous layer was recovered. Two volumes of ethanol was added thereto and cooled to −70° C. Then, the DNA was precipitated by centrifugation.

c) Phosphorylation of the 5′ End

The precipitate was dissolved in 10 μl of TE buffer (10 mM Tris-HCl (pH 8.0), 1 mM EDTA) and reacted in 100 μl of a phosphorylation reaction solution [50 mM Tris-HCl (pH 7.6), 10 mM MgCl₂, 1 mM spermidine, 10 mM dithiothreitol, 0.1 mg/ml bovine serum albumin, 1 mM ATP, 10 units of T4 polynucleotide kinase (Nippon Gene)] at 37° C. for 1 hr to phosphorylate the 5′ end. After phenol treatment, the aqueous layer was recovered. Two volumes of ethanol was added thereto and cooled to −70° C. Then, the DNA was precipitated by centrifugation and dissolved in 20 μl of TE buffer.

d) Construction of Expression Plasmid pTCIIZAQS

The NdeI-BamHI fragment from plasmid pTCII described in Example 12 and the ZAQ-1 ligand structural gene prepared as described above were subjected to a ligation reaction using TaKaRa DNA Ligation Kit ver. 2 (Takara Shuzo). Briefly, 1 μl of a solution of NdeI-BamHI fragment from pTCII and 4 μl of a solution of the ZAQ-1 ligand structural gene were mixed. To this mixture, 5 μl of solution I was added and reacted at 16° C. for 30 min for ligation. Using 10 μl of this ligation solution, E. coli JM109 competent cells (Yoyobo) were transformed, plated on LB agar medium containing 10 μg/ml tetracycline, and cultured at 37° C. for one day. The resultant tetracycline resistant colonies were selected. This transformant was cultured in LB medium overnight, followed by preparation of plasmid pTCIIZAQS using QIAprep8 Miniprep Kit (Qiagen). The nucleotide sequence of the ZAQ-1 ligand structural gene moiety was confirmed with Applied Biosystems Model 377 DNA Sequencer. Plasmid pTCIIZAQS was transformed into E. coli MM294 (DE3), which was then plated on LB agar medium containing 10 μg/ml tetracycline and cultured at 37° C. for one day. As a result, clone MM294 (DE3)/pTCIIZAQ was obtained that expresses a ZAQ-1 ligand having a nucleotide sequence comprising those codons highly frequently used in E. coli.

### Example 14

Search for ZAQ-1 Ligand Sequence Optimized for Expression

Plasmid pKANP constructed in Example 11 was digested with restriction enzymes NdeI (Takara Shuzo) and NgoMIV (New England BioLabs), and subjected to agarose gel electrophoresis to cut out an approx. 3.8 kbp band. The DNA was recovered from the gel using QIAquick Gel Extraction Kit (Qiagen).

Random nucleotide sequences encoding amino acids of an N-terminal portion of ZAQ-1 ligand were prepared as described below. Briefly, double-stranded DNA fragments were prepared with Pyrobest DNA polymerase (Takara Shuzo) using a 1:2 mixture of mixed bases-containing synthetic DNA 27: AGAGTTTGCCGGCACCGGTCCGGTACCCGCACCGCACTGCACRTCNCGYTCRCANGCNCCNGTDATNACNGCCATATGTTGGCGC (SEQ ID NO: 89) and synthetic DNA 28: AGAGTnTGCCGGCACCGGTCCGGTACCCGCACCGCACTGCACRTCYCTYTCRCANGCNCCNGTDATNACNGCCATATGTTGGCGC (SEQ ID NO: 90) as templates and synthetic DNA 29: GCGCCAACATATG (SEQ ID NO: 91). The extension reaction solution was composed of 3 μl of Pyrobest DNA polymerase (5 U/μl), 10 μl of 10× reaction buffer attached to the polymerase, 16 μl of dNTP mixture, 0.5 91 of synthetic DNA 35(1 μg/l), 1 μl of synthetic DNA 36(1 μg/μl), 2 μl of synthetic DNA 29 (100 μmol/L) and 67.5 μl of distilled water. The extension reaction was performed at 94° C. for 30 sec, at 58° C. for 30 sec and at 70° C. for 5 min. DNA fragments were purified from the reaction solution after the extension reaction using QIAquick Gel Extraction Kit, followed by digestion of the double-stranded DNA fragments with NdeI (Takara Shuzo) and NgoMIV (New England BioLabs). This digestion was performed as described below.

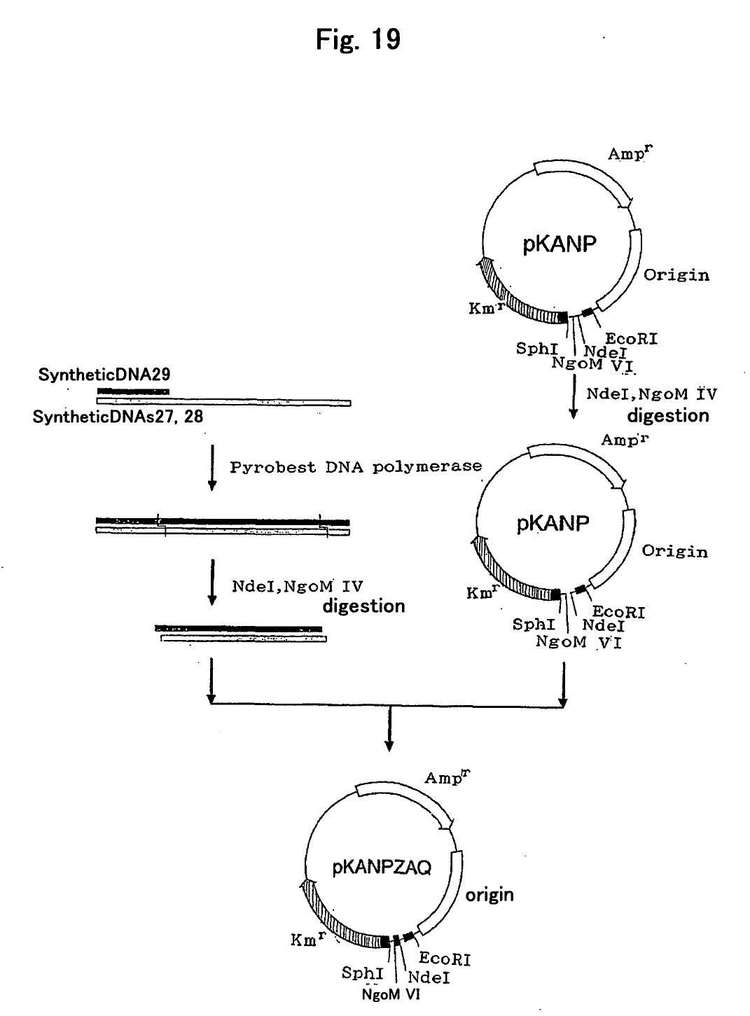

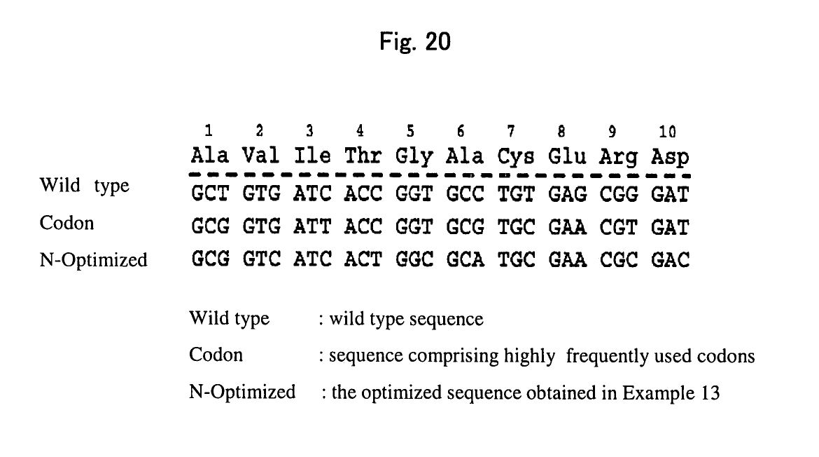

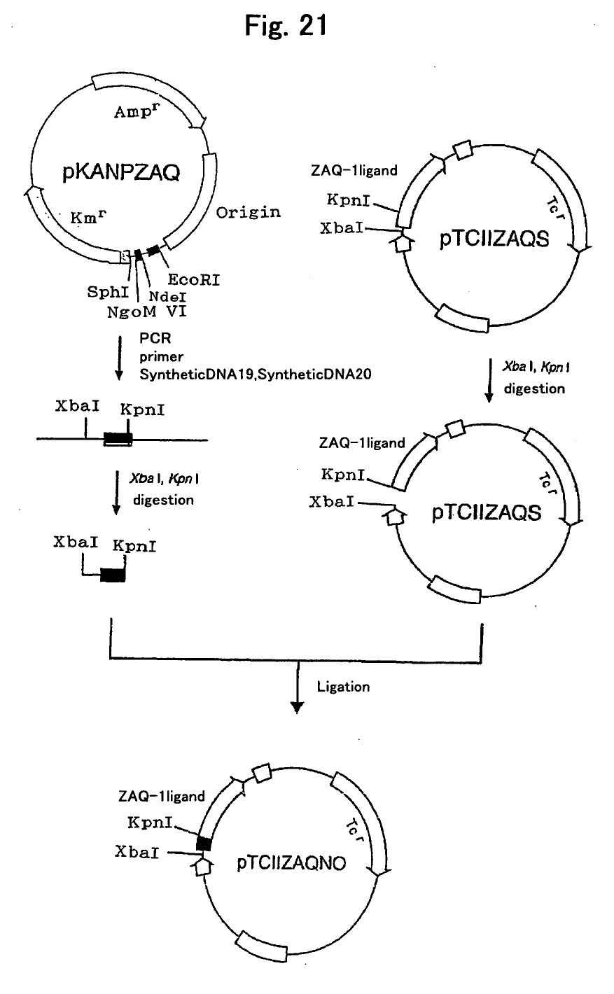

A reaction solution consisting of 27 μl of the purified DNA fragment solution, 3.5 μl of Buffer 4 (New England BioLabs), 1.5 μl of distilled water, 1.5 μl of NdeI and 1.5 μl of NgoMIV was retained at 37° C. for 2 hr to perform digestion. Then, an approx. 70 bp band was cut out through agarose gel electrophoresis, and the DNA was recovered from the gel using QIAquick Gel Extraction Kit (Qiagen). These DNA fragments were inserted into pKANP between the NdeI and NgoMIV sites using Ligation kit ver. 2 (Takara Shuzo) to construct a set of plasmid pKANPZAQ for searching for optimized nucleotide sequences (FIG. 19). E. coli MM294 was transformed with plasmid pKANPZAQ, plated on LB agar medium containing 50 μg/ml kanamycin and cultured at 37° C. for one day for colony formation. Resultant colonies were subjected to colony PCR using synthetic DNA 30: ACCTGATTGCCCGACATTATCGC (SEQ ID NO: 92) and synthetic DNA 31: CACCCTCATCAGTGCCAACATAG (SEQ ID NO: 93), and the insertion of a DNA of interest was confirmed by agarose gel electrophoresis. The product of this colony PCR was purified QIAquick PCR Purification Kit (Qiagen), and the nucleotide sequence thereof was analyzed with synthetic DNAs 30 and 31. As a result, the sequence shown in FIG. 20 was obtained.

### Example 15

Construction of Plasmid for Expressing ZAQ-1 Ligand Encoded by Expression Optimized Nucleotide Sequence

Expression plasmid pTCIIZAQS (obtained in Example 13) for a ZAQ-1 ligand encoded by a sequence with highly frequently used codons was digested with restriction enzymes XbaI (Takara Shuzo) and KpnI (Takara Shuzo), and subjected to agarose gel electrophoresis to cut out an approx. 4.8 kbp band. The DNA was recovered from the gel using QIAquick Gel Extraction Kit (Qiagen).

The product of colony PCR obtained in Example 14 was digested with restriction enzymes XbaI (Takara Shuzo) and KpnI (Takara Shuzo), and subjected to agarose gel electrophoresis to cut out an approx. 100 bp band. The DNA was recovered from the gel using QIAquick Gel Extraction Kit (Qiagen). This DNA fragment was inserted into pTCIIZAQS between the XbaI and KpnI sites using Ligation kit ver. 2 (Takara Shuzo) to thereby construct expression plasmid pTCIIZAQNO for a ZAQ-1 ligand encoded by a nucleotide sequence optimized for expression. The nucleotide sequence of the ZAQ-1 ligand structural gene moiety of this plasmid was confirmed with Applied Biosystems Model 3100 DNA Sequencer. The results revealed that this plasmid comprises a DNA (SEQ ID NO: 121) comprising the nucleotide sequence shown in the bottom row in FIG. 20 and yet encoding a ZAQ-1 peptide. Plasmid pTCIIZAQNO was transformed into E. coli MM294 (DE3). The resultant transformant was plated on LB agar medium containing 10 μg/ml tetracycline and cultured at 37° C. for one day to thereby obtain expression clone MM294 (DE3)/pTCIIZAQNO for ZAQ-1 ligand having a nucleotide sequence optimized for expression in E. coli.

### Example 16

Comparison of Expression Levels among ZAQ-1 Ligand Expression Clones

ZAQ-1 ligand expression clones MM294 (DE3)/pTCIIZAQN, MM294 (DE3)/pTCIIZAQS and MM294 (DE3)/pTCIIZAQNO obtained in Examples 12, 13 and 15 were cultured separately in 30 ml of LB medium (1% peptone, 0.5% yeast extract, 0.5% sodium chloride) containing 10 mg/L tetracycline at 30° C. for 16 hr. The resultant culture broth (1 ml each) was transferred to a 200 ml Erlenmeyer flask with pleats containing 19 ml of a medium for primary fermentation (1.68% sodium monohydrogenphosphate, 0.3% sodium dihydrogenphosphate, 0.1% ammonium chloride, 0.05% sodium chloride, 0.024% magnesium sulfate, 0.0005% thiamine hydrochloride, 1.5% glucose and 1.0% casamino 47 acids). Then, aeration agitation culture was started at 37° C. at 200 rpm. When the turbidity of the culture broth reached approx. 150 Klett units, isopropyl-β-D-thiogalacto-pyranoside (IPTG) was added at a final concentration of 0.1 mmol/L min. The cultivationwas continued up to 3 hr after the start of the addition of IPTG.

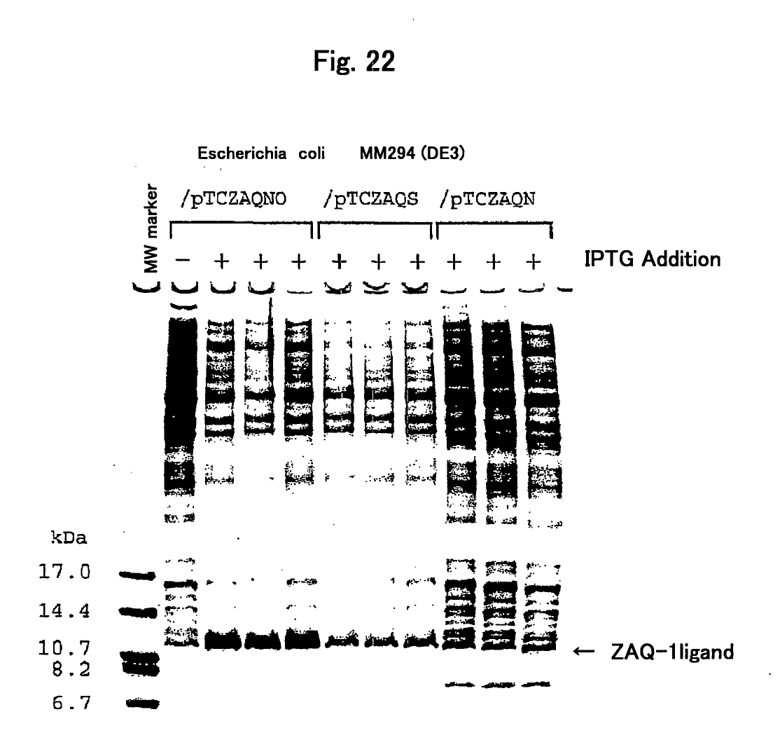

After completion of the cultivation, a 0.2 ml aliquot was taken from the culture broth and centrifuged at 12000 rpm for 10 min. The supernatant was discarded, and the resultant cells were suspended in a sample buffer (125 mM Tris-HCl, 1% sodium dodecyl sulfate, 15% glycerol, 5% 2-mercaptoethanol, 0.005% Bromophenol Blue) and treated at 95° C. for 5 min, followed by electrophoresis in Multigel 15/25 (Daiichi Pure Chemicals Co., Ltd.). The resultant gel was stained with Rapid CBB KANTO (Kanto Kagaku). As a result, ZAQ-1 ligand expression clone MM294 (DE3)/pTCIIZAQNO having a nucleotide sequence optimized for expression in E. coli exhibited a higher ZAQ-l ligand expression ability than E. coli MM294 (DE3)/pTCIIZAQN carrying an expression plasmid comprising a wild type nucleotide sequence encoding an N-terminal portion of ZAQ-1 ligand and ZAQ-1 ligand expression clone MM294 (DE3)/pTCIIZAQS having a nucleotide sequence comprising those codons used highly frequently in E. coli (FIG. 22). From these results, it has become clear that a structural gene using a nucleotide sequence found by the process of the invention is more suitable for expression than a structural gene using a nucleotide sequence comprising highly frequently used codons.

### Example 17

Construction of Rat GALP Direct Expression Plasmid (with Wild Type Nucleotide Sequence)

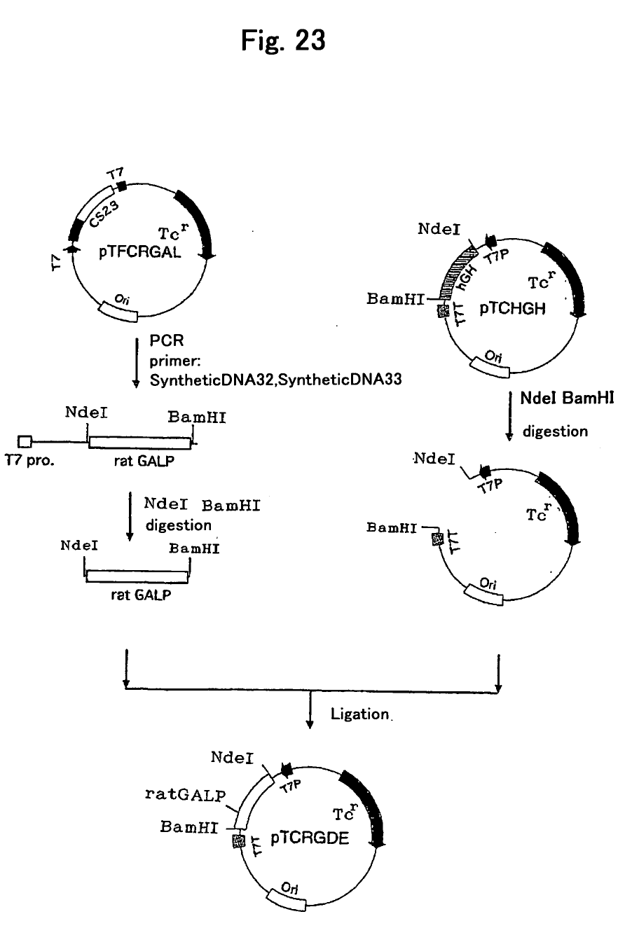

A rat GALP direct expression plasmid having a rat GALP structural gene consisting of the wild type nucleotide sequence was constructed as described below (FIG. 23).

Plasmid pTCHGH—Na described in Reference Example 1 in WO 00/20439 was digested with restriction enzymes NdeI and BamHI, and subjected to agarose gel electrophoresis to cut out an approx. 4.6 kbp band. The DNA was recovered from the gel using QIAquick Gel Extraction Kit (Qiagen) and used as an expression vector.

The GALP structural gene moiety was amplified from the rat GALP fusion protein expression plasmid pTFCRGAL obtained in Example 3. As a primer corresponding to the T7 promoter moiety of pTFCRGAL, synthetic DNA 32: TAATACGACTCACTATAGGG (SEQ ID NO: 94) was used. As a primer to introduce a stop codon and BamHI restriction site downstream of the C-terminal of rat GALP, synthetic DNA 33: ATACATGGATCCTCAGGTCATCCTTGGAGAGC (SEQ ID NO: 95) was used. The amplification was carried out in 100 μl of a reaction solution (containing 50 ng of plasmid pTFCRGAL, 200 pmol each of primers, 1 μl of Pyrobest DNA polymerase (Takara Shuzo), 8 μl of dNTP solution attached to the polymerase and 10 μl of ×10 reaction buffer attached to the polymerase) for 25 cycles, each cycle consisting of at 94° C. for 10 sec, at 55° C. for 30 sec and at 72° C. for 60 sec. The amplified DNA fragment was purified from the reaction solution using QIAquick PCR Purification Kit (Qiagen). The purified DNA fragment was reacted at 37° C. for 12 hr in 30 μl of a reaction solution containing 1 μl of NdeI (Takara Shuzo), 1 μl of BamHI (Takara Shuzo) and 5 μl of Buffer K (Takara Shuzo). After electrophoresis using 4% agarose gel, an approx. 0.2 kbp band was cut out, and the DNA was recovered from the gel using QIAquick Gel Extraction Kit (Qiagen). This DNA fragment was ligated to pTCHGH—Na between the NdeI and BamHI sites using Ligation kit ver. 2 (Takara Shuzo) to thereby obtain expression plasmid pTCRGDE for direct expression of rat GALP.

E. coli MM294 (DE3) was transformed with this expression plasmid pTCRGDE to thereby obtain E. coli MM 294 (DE3)/pTCRGDE.

### Example 18

Construction of Rat GALP Direct Expression Plasmid (Optimization of N-terminal Nucleotide Sequence)

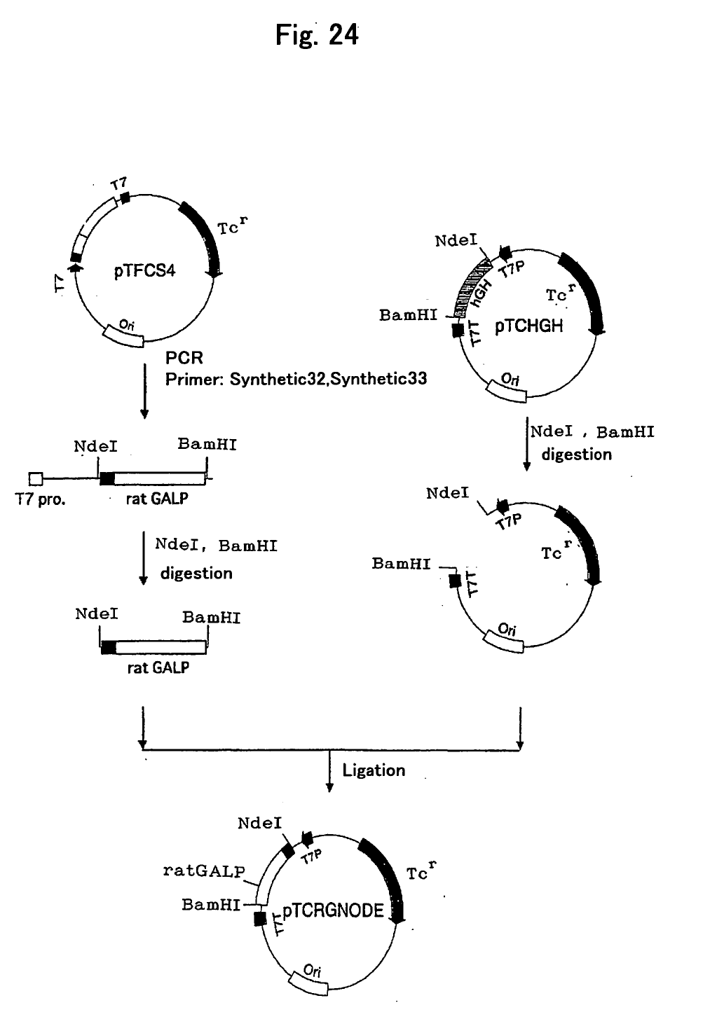

A rat GALP direct expression plasmid having a structural gene in which an N-terminal portion of rat GALP is replaced with the nucleotide sequence optimized for expression found in Example 2 was constructed as described below (FIG. 24).

Expression plasmid pTCRGNODE for direct expression of rat GALP was obtained in the same manner as in Example 17 except that the rat GALP fusion protein expression plasmid pTFCS4 obtained in Example 4 was used as a template. E. coli MM294 (DE3) was transformed with expression plasmid pTCRGNODE to thereby obtain E. coli MM294 (DE3)/pTCRGNODE.

### Example 19

Search for GALP Full-Length Sequence Optimized for Expression

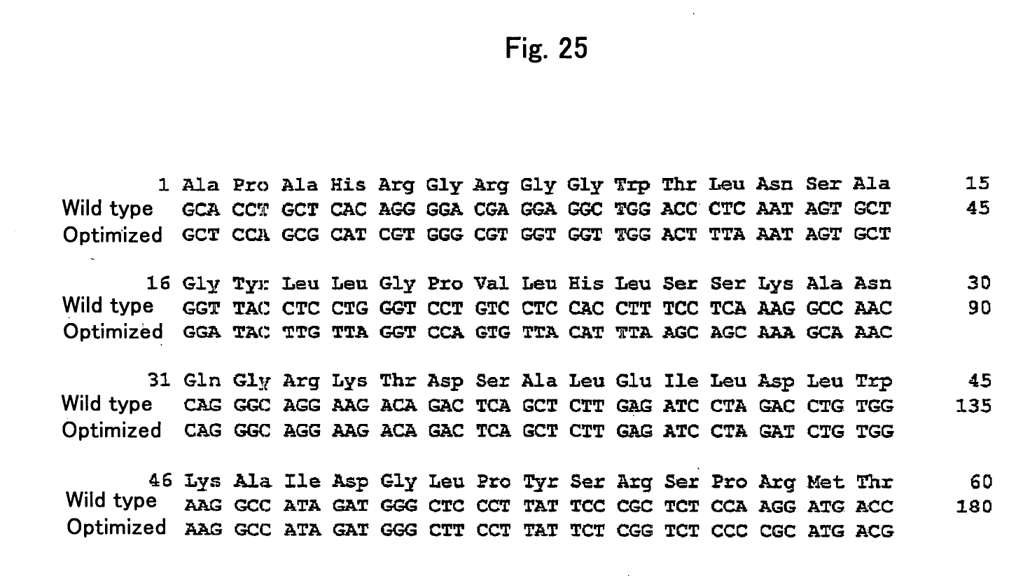

Search for optimized sequences was conducted not only for the nucleotide sequence encoding the N-terminal of GALP searched for in Example 2 but also for other nucleotide sequences encoding other parts. Briefly, the amino acid sequence of rat GALP consisting of 60 amino acids was divided into 4 blocks. Then, nucleotide sequences optimized for expression were searched for in the same manner as in Example 2 for the 3 blocks excluding the N-terminal block examined in Example 2. As a result, the sequence shown in FIG. 25 was obtained.

### Example 20

Construction of Expression Plasmid for GAL Encoded by the Full Length-Optimized Sequence

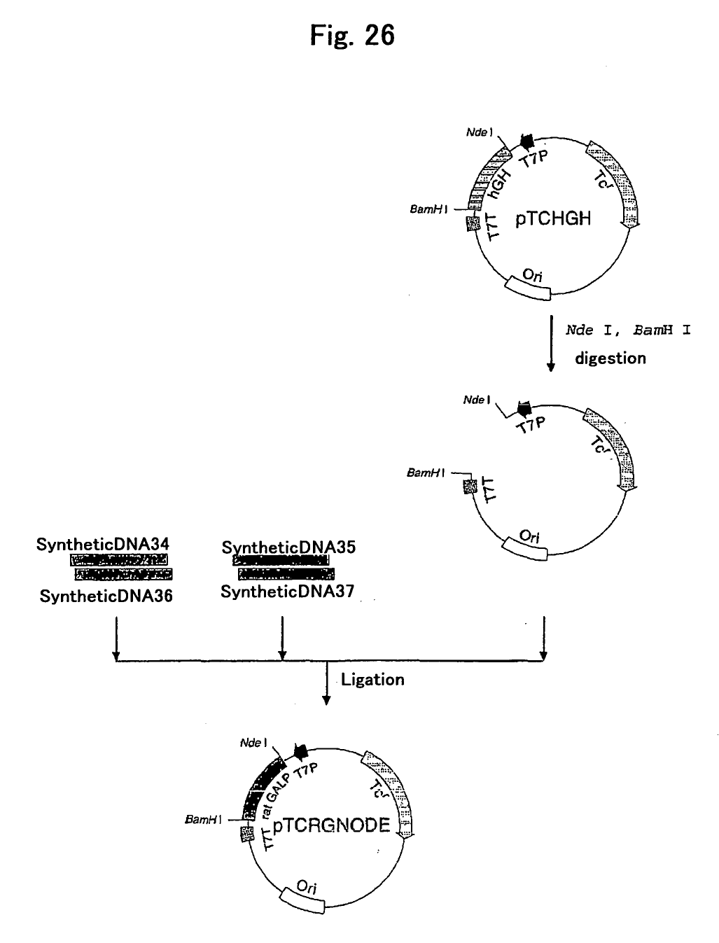

Expression plasmid for rat GALP having the nucleotide sequence found in Example 17 was constructed as described below (FIG. 26).

A rat GALP structural gene was constructed using synthetic DNA 34: TATGGCTCCAGCGCATCGTGGGCGTGGTGGTTGGACTTTAAATAGTGCTGGATACTTGTTAGGTCCAGTGTTACATMTAAGCAGCAAAGCAAACCAGGGC (SEQ ID 5 NO: 96), synthetic DNA 35: AGGAAGACAGACTCAGCTCTTGAGATCCTAGATCTGTGGAAGGCCATAGATGGGCTTCCTTATTCTCGGTCTCCCCGCATGACGTGAG (SEQ ID NO: 97), synthetic DNA 36: TTGCTTTGCTGCTTAAATGTAACACTGGACCTAACAAGTATCCAGCACTATTTAAAGTCCAACCACCACGCCCACGATGCGCTGGTGCCA (SEQ ID NO: 98) and synthetic DNA 37: GATCCTCACGTCATGCGGGGAGACCGAGAATAAGGAAGCCCATCTATGGCCTTCCACAGATCTAGGATCTCAAGAGCTGAGTCTGTCTTCCTGCCCTGGT (SEQ ID NO: 99). Briefly, synthetic DNAs 34 to 37 were dissolved separately in TE buffer to give a concentration of 50 mmol/L. Three μl of each of the synthetic DNA solution was reacted in a phosphorylation reaction solution [50 mM Tris-HCl (pH 7.6), 10 mM MgCl₂, 1 mM spermidine, 10 mM dithiothreitol, 0.1 mg/ml bovine serum albumin, 1 mM ATP, 10 units of T4 polynucleotide kinase (Nippon Gene)] for 1 hr at 37° C. to phosphorylate the 5′ end. The phosphorylated synthetic DNAs 34 and 36 or synthetic DNAs 35 and 37 were mixed, retained at 90° C. for 10 min and then gradually cooled to room temperature for annealing. Each mixture was subjected to agarose gel electrophoresis to cut out an approx. 0.1 kbp band, and the DNA was recovered from the gel using QIAquick Gel Extraction Kit (Qiagen). Thus, two double-stranded DNA fragments representing the N-terminal half and C-terminal half of rat GALP were obtained.

The NdeI-BamHI fragment from pTCHGH—Na described in Example 17 was mixed with the above-obtained two double-stranded DNA fragments representing the N-terminal half and C-terminal half of rat GALP and ligated together using Ligation kit ver. 2 (Takara Shuzo) to thereby construct plasmid pTCRGTODE for direct expression of rat GALP encoded by a full length-optimized nucleotide sequence. This plasmid comprises a DNA (SEQ ID NO: 122) comprising the nucleotide sequence shown at the bottom row in FIG. 25 and having a nucleotide sequence encoding rat GALP. E. coli MM294 (DE3) was transformed with this expression plasmid pTCRGTODE to thereby obtain E. coli MM294 (DE3)/pTCRGTODE.

### Example 21

Direct Expression of GALP Encoded by the Full Length-Optimized Sequence

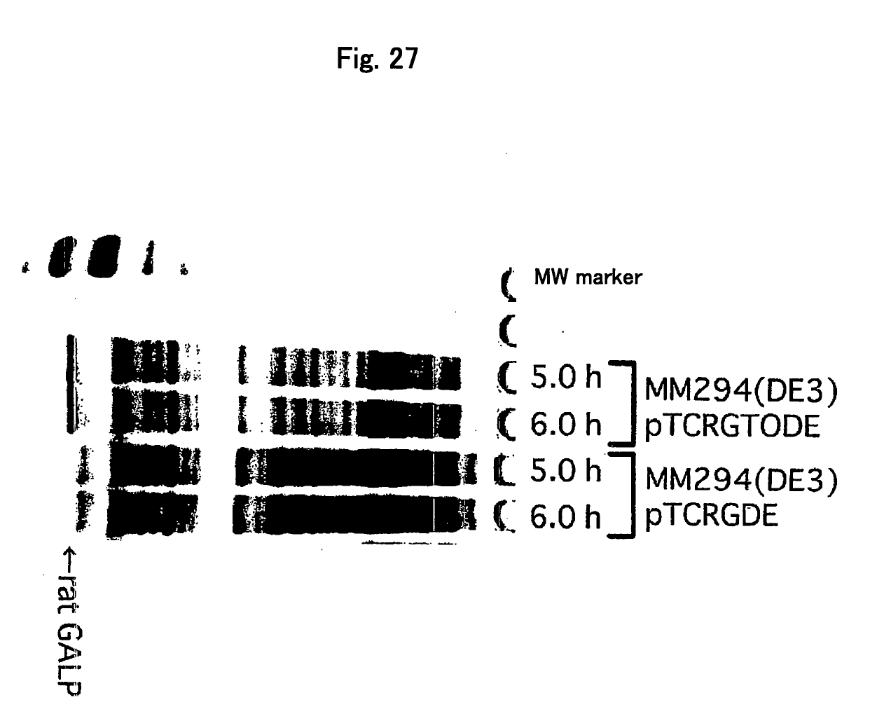

E. coli MM294 (DE3)/pTCRGDE obtained in Example 17, E. coli MM294 (DE3)/pTCRGNODE obtained in Example 18 and E. coli MM294 (DE3)/pTCRGTODE obtained in Example 20 were cultured separately in 1 L of LB medium (1% peptone, 0.5% yeast extract, 0.5% sodium chloride) containing 10 mg/L tetracycline at 30° C. for 16 hr. The resultant culture broth (75 ml each) was transferred to a 3-L jar fermenter containing 1.5 L of a medium for primary fermentation (1% peptone, 0.5% yeast extract, 0.5% sodium chloride, 0.02% New Pole LB-625). Then, aeration agitation culture was started at 37° C. under a gas flow of 1.5 lUmin at an agitation speed of 550 rpm. When the turbidity of the culture broth reached approx. 150 Klett units, isopropyl-β-D-thiogalactopyranoside (IPTG) was added at 50 μmol/L min. A 0.2 ml sample was taken from the culture broth at regular intervals during the cultivation and centrifuged at 12000 rpm for 10 min. The supernatant was discarded, and the resultant cells were suspended in a sample buffer (125 mM Tris-HCl, 1% sodium dodecyl sulfate, 15% glycerol, 5% 2-mercaptoethanol, 0.005% Bromophenol Blue) and treated at 95° C. for 5 min, followed by electrophoresis in Multigel 15/25 (Daiichi Pure Chemicals Co., Ltd.). The resultantgel was stained with Rapid CBB KANTO (Kanto Kagaku). As a result, a band of rat GALP was not observed on the SDS-PAGE in the lanes of E. coli MM294 (DE3)/pTCRGDE carrying an expression plasmid whose rat GALP structural gene entirely consists of the natural type nucleotide sequence and rat GALP expression clone E. coli MM294 (DE3)/pTCRGNODE having a nucleotide sequence optimized only in an N-terminal portion. A band of rat GALP was recognized in the lane of rat GALP expression clone E. coli MM294 (DE3)/pTCRGTODE having a nucleotide sequence optimized for the full-length of rat GALP structural gene for the purpose of expression in E. coli. FIG. 27 shows the results for MM294 (DE3)/pTCRGDE and MM294 (DE3)/pTCRGTODE. Even when direct expression of a protein encoded by the wild-type nucleotide sequence is difficult in the expression of relatively low molecular weight proteins as seen in this case, it can be expected to make direct expression possible by optimizing the nucleotide sequence for expression according to the process of the invention. Further, it has been demonstrated that high expression is possible by optimizing the nucleotide sequence for the full length of a structural gene when expected expression cannot be obtained by optimizing the nucleotide sequence encoding an N-terminal portion of the gene.

### Example 22

Construction of Plasmids for Measuring Expression Efficiencies in Animal Cells

1) Construction of Plasmid pQBI25-2

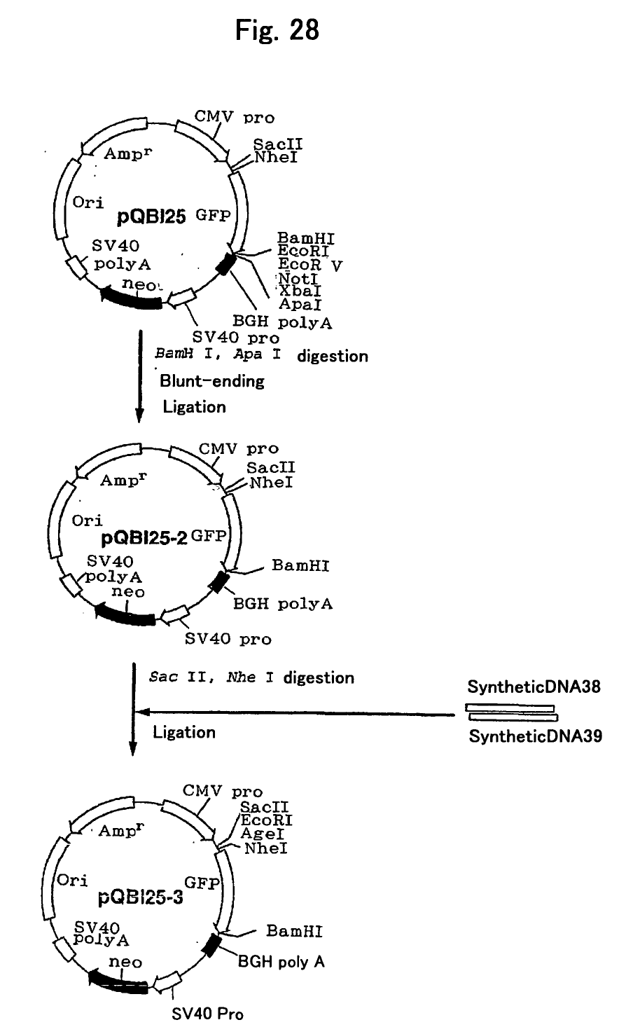

Plasmid pQBI25 (Takara Shuzo) was digested with BamHI (Takara Shuzo) and ApaI (Takara Shuzo) in order to eliminate the EcoRI, EcoRV, NotI, XbaI and ApaI restriction sites located within the multicloning site downstream of the GFP structural gene, followed by blunt-ending using DNA Blunting Kit (Takara Shuzo). Subsequently, an approx. 6.1 kbp band was cut out by agarose gel electrophoresis. The DNA was recovered from the gel using QIAquick Gel Extraction Kit (Takara Shuzo), followed by self-ligation using Ligation Kit Version 2 (Takara Shuzo) to thereby obtain plasmid pQBI25-2. Plasmid pQBI25-2 was digested with SacII (Takara Shuzo) and NheI (Takara Shuzo), and an approx. 6.1 kbp band was cut out by agarose gel electrophoresis. The DNA was recovered from the gel using QIAquick Gel Extraction Kit (Takara Shuzo). One hundred pmol each of synthetic DNA 38: GGAATTCGCTCGCACCGGTAGAAAAAATGG (SEQ ID NO: 100) comprising EcoRI and AgeI restriction sites and synthetic DNA 39: CTAGCCATTTTTTCTACCGGTGCGAGCGAATTCCGC (SEQ ID NO: 101) comprising a nucleotide sequence complementary thereto were dissolved in TE buffer, retained at 90° C. for 10 min, and then gradually cooled to room temperature for annealing. The resultant double-stranded DNA fragment was inserted into pQBI25-2 between the ScaII and NheI sites using Ligation Kit Version 2 (Takara Shuzo) to thereby obtain plasmid pQBI25-3 (FIG. 28).

Plasmid pQBI25-3 is an expression plasmid that comprises a GFP structural gene transcribed from CMV promoter in eukaryotic cells and has SacII, EcoRI and AgeI restriction sites upstream of the GFP structural gene. A DNA fragment can be inserted into this plasmid using these restriction sites.

### Example 23

Construction of Expression Plasmid for Comparing Expression Levels of GFP Proteins with Different Nucleotide Sequences encoding N-Terminal Amino Acids

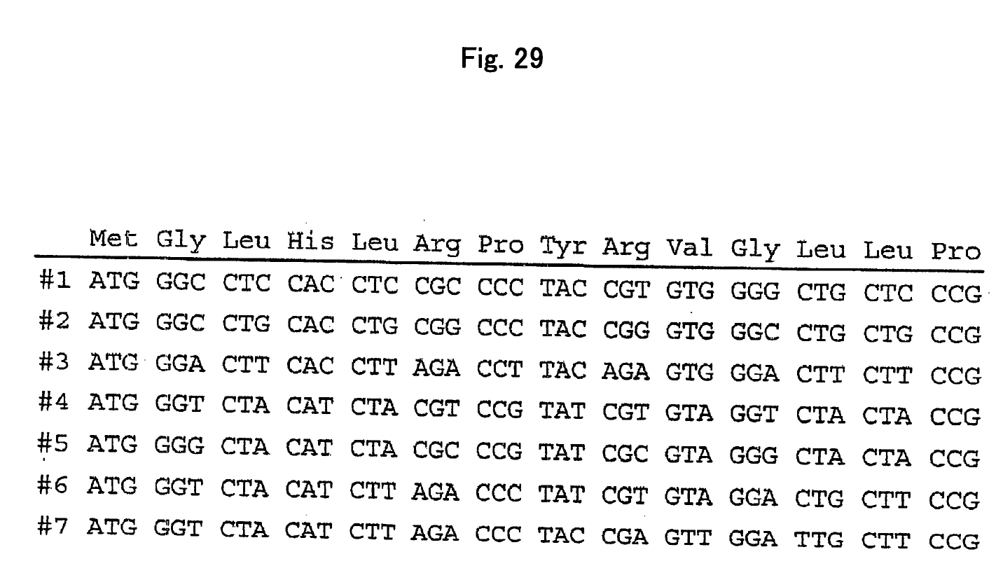

A set of expression plasmids that have seven nucleotide sequences each encoding the amino acid sequence from the N-terminal to the 13th residue of human LLPL (lecithin cholesterol acyltransferase-like lysophospholipase, Biochem Biophys Res Commun. Vo. 257, 50 (1999)) upstream of their GFP structural gene were prepared as described below. Sequence #1 represents the wild type nucleotide sequence (FIG. 29).

Plasmid pQGI-25-3 obtained in Example 22 was digested with EcoRI (Takara Shuzo) and AgeI (Takara Shuzo), and an approx. 6.1 kbp band was cut out by agarose gel electrophoresis. The DNA was recovered from the gel using QIAquick Gel Extraction Kit (Takara Shuzo).

The following synthetic DNAs (50 pmol each) were dissolved in TE buffer separately, and their 5′ ends were phosphorylated with T4 Polynucleotide Kinase (Wako Purechemical Industries). Sequence #1 was obtained by mixing the 5′ end phosphorylation reaction solutions of synthetic DNA 40: AATTCCGTCGTACACCATGGGCCTCCACCTCCGCCCCTACCGTGTGGGGCTGCT C (SEQ ID NO: 102) and synthetic DNA 41: CCGGGAGCAGCCCCACACGGTAGGGGCGGAGGTGGAGGCCCATGGTGTACGACGG (SEQ ID NO: 103) comprising a nucleotide sequence complementary thereto, retaining the mixture at 90° C. for 10 min and gradually cooling to room temperature for annealing. The resultant double-stranded DNA fragment was ligated into plasmid pQBI25-3 between the EcoRI and AgeI sites using Ligation Kit Version 2 (Takara Shuzo) to thereby obtain plasmid pQBI25-3LLPL#1. Sequence #2 was prepared from synthetic DNA 42: AATTCCGTCGTACACCATGGGCCTGCACCTGCGGCCCTACCGGGTGGGCCTGCTG (SEQ ID NO: 104) and synthetic DNA 43: CCGGCAGCAGGCCCACCCGGTAGGGCCGCAGGTGCAGGCCCATGGTGTACGACGG (SEQ ID NO: 105) comprising a nucleotide sequence complementary thereto; sequence #3 was prepared from synthetic DNA 44: AATTCCGTCGTACACCATGGGACTTCACCTTAGACCTTACAGAGTGGGACTTCTT (SEQ ID NO: 106) and synthetic DNA 45: CCGGAAGAAGTCCCACTCTGTAAGGTCTAAGGTGAAGTCCCATGGTGTACGACGG (SEQ ID NO: 107) comprising a nucleotide sequence complementary thereto; sequence #4 was prepared from synthetic DNA 46: AATTCCGTCGTACACCATGGGTCTACATCTACGTCCGTATCGTGTAGGTCTACTA (SEQ ID NO: 108) and synthetic DNA 47: CCGGTAGTAGACCTACACGATACGGACGTAGATGTAGACCCATGGTGTACGACGG (SEQ ID NO: 109) comprising a nucleotide sequence complementary thereto; sequence #5 was prepared from synthetic DNA 48: AATTCCGTCGTACACCATGGGGCTACATCTACGCCCGTATCGCGTAGGGCTACTA (SEQ ID NO: 110) and synthetic DNA 49: CCGGTAGTAGCCCTACGCGATACGGGCGTAGATGTAGCCCCATGGTGTACGACGG (SEQ ID NO: 111) comprising a nucleotide sequence complementary thereto; sequence #6 was prepared from synthetic DNA 50: AATTCCGTCGTACACCATGGGTCTACATCTTAGACCCTATCGTGTAGGACTGCTT (SEQ ID NO: 112) and synthetic DNA 51: CCGGAAGCAGTCCTACACGATAGGGTCTAAGATGTAGACCCATGGTGTACGACGG (SEQ ID NO: 113) comprising a nucleotide sequence complementary thereto; sequence #7 was prepared from synthetic DNA 52: AATTCCGTCGTACACCATGGGTCTACATCTTAGACCCTACCGAGTTGGATTGCTT (SEQ ID NO: 114) and synthetic DNA 53: CCGGAAGCAATCCAACTCGGTAGGGTCTAAGATGTAGACCCATGGTGTACGACGG (SEQ ID NO: 115) comprising a nucleotide sequence complementary thereto; and inserted into the plasmid in the same manner as described for sequence #1 to thereby obtain plasmids pQBI25-3#2 through pQBI25-3#7.

### Example 24

Comparison of Expression Levels of GFP Proteins with Different Nucleotide Sequences in the N-terminal in Temporary Expression in Animal Cells

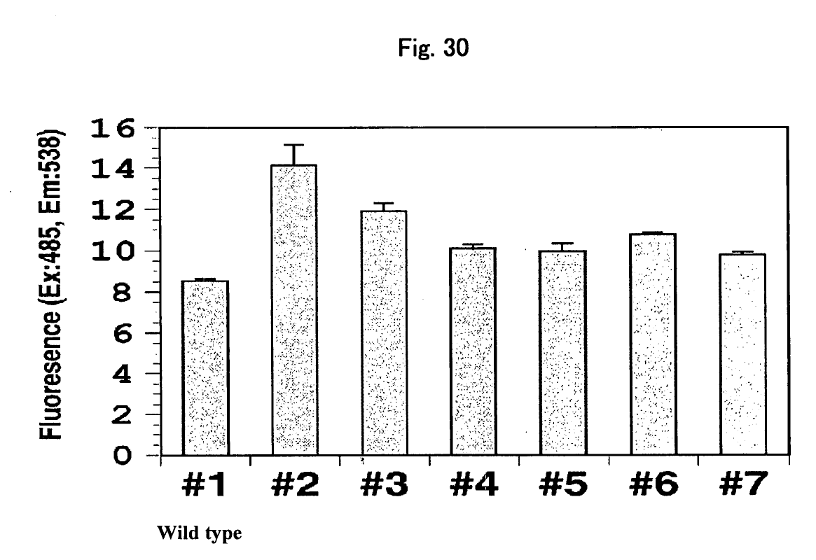

COS-7 cells cultured in 24-well plates were transformed with plasmids pQBI25-3#1 through pQBI25-3#7 obtained in Example 23 using LipofectAmine PLUS (Gibco BRL) according to the protocol attached thereto. Forty-two hours after the transformation, fluorescence intensity at the excitation wavelength of 435 nm and emission wavelength of 538 nm was measured with a multiplate fluorescence measurement apparatus (Dainippon Pharmaceutical). As a result, the nucleotide sequences #2 through #7 generally exhibited higher fluorescence than sequence #1 having the N-terminal nucleotide sequence of the wild-type LLPL. The results also revealed that the expression levels of GFP (reporter gene) vary depending on the nucleotide sequence encoding the N-terminal portion of LLPL (FIG. 30).

### Example 25

Comparison of Expression Levels of GFP Proteins with Different Nucleotide Sequences in the N-terminal in Stable Expression in Animal Cells

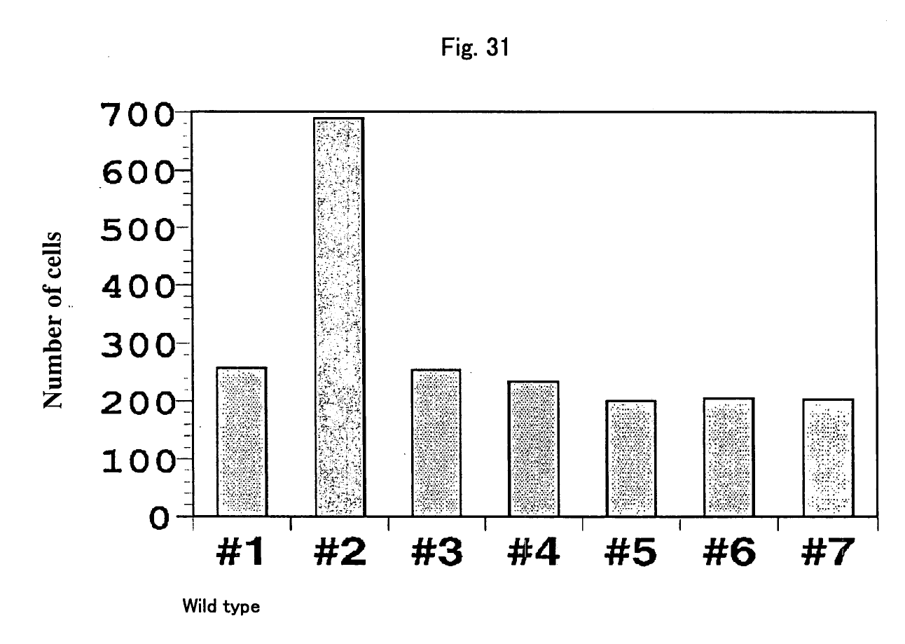

Plasmids pQBI25-3LLPL#1 through pQBI25-3LLPL#7 obtained in Example 23 were cut at the ScaI site located in the ampicillin resistance gene with ScaI (Takara Shuzo) to obtain linear DNAs. CHO-K1 cells cultured in a usual manner were scraped off, washed with PBS and suspended in PBS to give a concentration of 1×10⁷ cells/0.8 ml. Ten μg each of the linear DNAs from plasmids pQBI25-3LLPL#l through pQBI25-3LLPL#7 were added separately to the cell suspension, which was then left stationary in ice for 5 min. Subsequently, transformation was carried out at 960 μF and 250 V with an electroporation apparatus (Bio-Rad). The cells were left stationary in ice for 10 min, then returned to a medium and cultured. Thirty hours after the transformation, the cells were transferred to a medium supplemented with geneticin (Gibco BRL) at 0.8 mg/ml and cultured further to select resistant clones. Thirteen days after the transformation, the cells were scraped off and subjected to measurement. Briefly, using a flowcytometer (Falcon), the fluorescence of individual cells excited by argon laser of 488 nm was measured with a detector equipped with a 530 nm filter. For each type of the transformants obtained with the above-described expression plasmids, 10,000 cells were measured. Then, GFP expression levels were compared using the number of cells whose fluorescence intensity (measured value) is 101 or above when the voltage of the photomultiplier tube in the detector was set at 500 V As a result, when sequences #3 through #7 were used, the number of such cells was equal to or below that number when sequence #1 (having the N-terminal nucleotide sequence of the wild-type LLPL) was used. When sequence #2 was used, the number of such cells was more than 2 fold of that number when sequence #1 was used. It was shown that the expression levels of GFP (reporter gene) vary depending on the nucleotide sequence encoding the N-terminal portion of LLPL even in stable expression in animal cells (FIG. 31).

### INDUSTRIAL APPLICABILITY

According to the present invention, a protein of interest can be produced in a large quantity using recombinant DNA technology. Thus, the present invention is useful as a process for industrial production of a protein of interest. Furthermore, according to the present invention, a transformant producing a protein of interest in a large quantity can be obtained easily and simply.

1. A process of producing a DNA that encodes a protein of interest and is suitable for expression, comprising the following steps:
1) preparing recombinant vectors by using a mixture of a plurality of DNAs encoding the full length or a partial amino acid sequence of the protein of interest and integrating each of said DNAs into a vector retaining a promoter DNA functional in a host cell and a reporter gene so that said DNA is located downstream of the promoter DNA and upstream of the reporter gene,
2) transforming the host cell with the recombinant vectors,
3) culturing the resultant transformants,
4) measuring the expression of the reporter gene, and
5) preparing a DNA that comprises the same nucleotide sequence as that of the DNA encoding the full length or the partial amino acid sequence of the protein of interest possessed by a transformant in which a high expression of the reporter gene has been confirmed and that encodes the protein of interest.

2. The process according to claim 1, wherein the full length or a partial amino acid sequence of the protein of interest is the full length of the protein of interest or a partial amino acid sequence comprising the N-terminal of the protein of interest.

3. A process of producing a protein of interest or a salt thereof, comprising the following steps:
1) preparing recombinant vectors by using a mixture of a plurality of DNAs encoding the full length or a partial amino acid sequence of the protein of interest and integrating each of said DNAs into a vector retaining a promoter DNA functional in a host cell and a reporter gene so that said DNA is located downstream of the promoter DNA and upstream of the reporter gene,
2) transforming the host cell with the recombinant vectors,
3) culturing the resultant transformants,
4) measuring the expression of the reporter gene,
5) preparing a recombinant vector comprising a DNA that comprises the same nucleotide sequence as that of the DNA encoding the full length or the partial amino acid sequence of the protein of interest possessed by a transformant in which a high expression of the reporter gene has been confirmed and that encodes the protein of interest,
6) transforming a host cell with the recombinant vector, and
7) culturing the resultant transformant.

4. The process according to claim 2, wherein the full length or a partial amino acid sequence of the protein of interest is the full length of the protein of interest or a partial amino acid sequence comprising the N-terminal of the protein of interest.

5. A process of producing a DNA that encodes a eukaryote-derived protein of interest and is suitable for expression in a prokaryote, comprising the following steps:
1) preparing recombinant vectors by using a mixture of a plurality of DNAs encoding an N-terminal amino acid sequence of the eukaryote-derived protein of interest and integrating each of said DNAs into a vector retaining a promoter DNA functional in prokaryotes and a reporter gene so that said DNA is located downstream of the promoter DNA and upstream of the reporter gene,
2) transforming the prokaryote with the recombinant vectors,
3) culturing the resultant prokaryotic transformants,
4) measuring the expression of the reporter gene, and
5) preparing a DNA that comprises the same nucleotide sequence as that of the DNA encoding the N-terminal amino acid sequence of the protein of interest possessed by a transformant in which a high expression of the reporter gene has been confirmed and that encodes the protein of interest.

6. A process of producing an eukaryote-derived protein of interest or a salt thereof in a prokaryote, comprising the following steps:
1) preparing recombinant vectors by using a mixture of a plurality of DNAs encoding an N-terminal amino acid sequence of the eukaryote-derived protein of interest and integrating each of said DNAs into a vector retaining a promoter DNA functional in prokaryotes and a reporter gene so that said DNA is located downstream of the promoter DNA and upstream of the reporter gene,
2) transforming the prokaryote with the recombinant vectors,
3) culturing the resultant prokaryotic transformants,
4) measuring the expression of the reporter gene,
5) preparing a recombinant vector comprising a DNA that comprises the same nucleotide sequence as that of the DNA encoding the N-terminal amino acid sequence of the protein of interest possessed by a transformant in which a high expression of the reporter gene has been confirmed and that encodes the protein of interest,
6) transforming the prokaryote with the recombinant vector, and
7) culturing the resultant prokaryotic transformant.

7. A non-wild type DNA obtained by the production process according to claim 1, that comprises the same nucleotide sequence as that of the DNA encoding the full length or a partial amino acid sequence of a protein of interest possessed by a transformant in which a high expression of the reporter gene has been confirmed and that encodes the full length of the protein of interest.

8. A non-wild type DNA obtained by the production process according to claim 1 encoding the full length of a protein of interest, wherein the DNA is composed of a DNA encoding a partial amino acid sequence of the protein of interest possessed by a transformant in which a high expression of the reporter gene has been confirmed and a DNA(s) encoding the remaining amino acid sequence(s) of said protein ligated to the 3′ end or/and 5′ end of said DNA.

9. A DNA having the nucleotide sequence as shown in SEQ ID NO: 119, SEQ ID NO: 120, SEQ ID NO: 121 or SEQ ID NO: 122.

10. A recombinant vector comprising the DNA according to any one of claims 7 to 9.

11. A transformant transformed with the recombinant vector according to claim 10.

12. A process of producing a protein of interest or a salt thereof, comprising culturing the transformant according to claim 11 and allowing the transformant to produce the protein of interest.
